(19) United States
(12) Patent Application Publication
(10) Pub. No.: US 2021/0317508 A1
(43) Pub. Date: Oct. 14, 2021

(54) METHODS AND APPARATUS FOR DETERMINING RISK OF AUTISM SPECTRUM DISORDER

(71) Applicants: Axial Therapeutics, Inc., Woburn, MA (US); California Institute of Technology, Pasadena, CA (US)

(72) Inventors: Gil SHARON, Los Angeles, CA (US); Sarkis MAZMANIAN, Glendale, CA (US); Ryan BARRETT, Acton, MA (US); Anthony Stewart CAMPBELL, Framingham, MA (US)

(21) Appl. No.: 16/635,752

(22) PCT Filed: Jul. 30, 2018
(86) PCT No.: PCT/US2018/044433

§ 371 Date: Jan. 31, 2020

Related U.S. Application Data
(60) Provisional application No. 62/539,958, filed on Aug. 1, 2017.
(60) Provisional application No. 62/614,187, filed on Jan. 5, 2018.

(51) Int. Cl. C12Q 1/689; G01N 33/68

(52) U.S. Cl. CPC C12Q 1/689; G01N 2333/195; G01N 2800/50; G01N 33/6896

(57) ABSTRACT

Aspects of the invention described herein relate to bacteria and metabolites, which may be used in diagnostics, therapeutics and/or dietary supplements so as to identify the risk to an individual of developing ASD or ASD-like disorders, as well as, to inhibit, treat, and/or prevent the onset of ASD and/or and ASD-like disorders.

### INCORPORATION BY REFERENCE TO ANY PRIORITY APPLICATIONS

Any and all applications for which a foreign or domestic priority claim is identified in the Application Data Sheet (including any PCT Request) as filed with the present application are hereby incorporated by reference under 37 CFR 1.57. This application claims the benefit of U.S. Provisional Application No. 62/539,958, filed Aug. 1, 2017, and of U.S. Provisional Application No. 62/614,187, filed Jan. 5, 2018, each of which is hereby incorporated by reference in its entirety.

### STATEMENT REGARDING FEDERALLY SPONSORED R&D

This invention was made with government support under MH100556 awarded by the National Institutes of Health. The government has certain rights in the invention.

### REFERENCE TO SEQUENCE LISTING AND TABLES IN ELECTRONIC FORMAT

This application is filed with an electronic sequence listing entitled AXLB009WOSEQLIST.TXT, created on Jul. 30, 2018 which is 6,154 bytes in size. The information in the electronic sequence listing is hereby incorporated by reference in its entirety.

### FIELD

Some embodiments described herein relate generally to compositions, products, and methods for the detection of autism spectrum disorder, as well as, additional neurological, neurodevelopmental, and/or neurodegenerative disorders.

### BACKGROUND

Autism spectrum disorder (ASD) is a devastating disease with a wide spectrum of manifestations that involve several behavioral deficits, including abnormal social interaction and communication and stereotypic behavior. The rates of diagnosis have increased dramatically over the past decades. As of 2013, one in every 68 children is diagnosed with ASD (an increase from 1 in 150 in 2002); this increase may partially be due to revised diagnostic criteria and increased awareness, however, unknown environmental factors appear to contribute in ways that remain undefined. While the genetic basis for ASD has received much attention over the past decade, recent evidence indicates that an interaction between genetics and environment may underlie certain behavioral abnormalities. Numerous environmental contributors may play a role in potentiating and/or causing symptoms in subjects with a genetic predisposition to ASD.

A non-genetic (yet heritable) component of human biology is the microbiome, a myriad of indigenous bacteria that have been implicated in immunologic, metabolic, and more recently neurologic, diseases. Reflecting a growing crisis in Western societies, recent epidemiologic reports have revealed dramatic increases in the incidence of several human disorders including e.g., inflammatory bowel disease (IBD), obesity, asthma, type-1 diabetes and multiple sclerosis (MS). A wealth of studies has now shown that IBD, asthma and obesity have a strong connection to the human gut microbiota. Several investigators have also shown that gut bacteria are a critical component of MS, anxiety and depression in animal models.

Often ASD subjects have gastrointestinal (GI) abnormalities, and a strikingly high incidence of intestinal barrier dysfunction is found in ASD children. Additionally, an association between GI dysfunction and the severity of ASD symptoms was found. Consistent with these GI features, the composition of gut bacteria is altered in children with ASD compared to controls, a feature exacerbated in those with GI complications. However, as these studies often show correlation rather than causation, it is hard to know whether the change in gut bacterial communities is a cause or an effect. Accordingly there is a need to identify correlations between ASD and ASD-like symptoms and elements, residents, and biomarkers, such that said correlations may be used to identify and/or predict the risk of developing ASD and/or ASD-like symptoms in an individual.

### SUMMARY

It has been discovered that the microbial and metabolic signatures in ASD-colonized mice, compared to that of NT-colonized mice (wild-type mice colonized with bacteria from neurotypical children), provide diagnostic biomarkers, which can be used to characterize, classify, and/or diagnose a subset of ASD patients (and possibly mothers during pregnancy) via analysis of patient biological samples (e.g., fecal, urine, and blood samples) of patients. Bacteria and metabolites enriched in NT-colonized mice may be used as therapeutics or dietary supplements, e.g., either as probiotics (for bacteria) or as drugs (metabolites) during gestation or later in life. Accordingly, several methods have been devised, which utilize these diagnostic biomarkers for determining or evaluating the risk to an individual of developing ASD or ASD-like disorders, as well as, an apparatus for utilizing the methods and biomarkers disclosed herein for identifying the risk of an individual developing ASD or and ASD-like disorders. The methods and devices of the present disclosure, preferably, comprise the following alternatives and such equivalents as could be reasonably envisioned by one of skill in the art.

In some embodiments a composition comprising a binding target joined specifically to a first binding agent is described. The binding target can be from one or more bacteria listed in Table 1A.1-1A.2 and/or Table 1B.1-1B.2. In some embodiments, the binding target is from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/ruminococcaceae, parabacteroides, and/or eisenbergiela. In some embodiments, the binding target comprises a second nucleic acid, and the first binding agent comprises a first nucleic acid complementary to the second nucleic acid. The second nucleic acid can be specific to one or more bacteria listed in Table 1A.1-1A.2 and/or Table 1B.1-1B.2. In some embodiments, the second nucleic acid is specific to Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/ruminococcaceae, or a combination of two or more of these. In some embodiments, the second nucleic acid is specific to Bacteroides ovatus, Parabacteroides merdae, Bacteroides thetaiotaomicron, Eisenbergiela tayi, or a combination of two or more of these. In some embodiments, the binding target of the composition consists essentially of nucleic acid from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/ruminococcaceae, parabacteroides, and/or eisenbergiela. In some embodiments, the second nucleic acid comprises a sequence encoding a 16S RNA. In some embodiments, the second nucleic acid comprises a sequence other than a 16S RNA coding sequence. In some embodiments, (a) the second nucleic acid is specific to Bacteroides ovatus, and comprises an sOTU sequence of sOTU b20cd_Bacteroides; or (b) the second nucleic acid is specific to Parabacteroides merdae, and comprises an sOTU sequence of sOTU 4ae7e_Parabacteroides; or (c) the second nucleic acid is specific to Eisenbergiela tayi, and comprises an sOTU sequence of sOTU 02b40_Lacnospiraceae and/or 29857_Lacnospiraceae; or (a) and (b); or (a) and (c); or (b) and (c); or (a), (b), and (c). In some embodiments, the second nucleic acid comprises one or more of SEQ ID NOs: 1-20, or at least 10 consecutive nucleotides thereof, for example at least 10, 15, 20, 25, 30, 35, 40, 45, or 50 nucleotides, including ranges between any two of the listed values, for example 10-50, 10-40, 10-30, 10-20, 20-50, 20-40, 20-30, 30-50, 30-40, or 40-50. In some embodiments, the binding target comprises an isolated antigen, and the first binding agent comprises an antibody, protein, peptide, or aptamer. In some embodiments, the first binding agent is specific to one or more bacteria of Tables 1A.1-1A.2 and/or Table 1B.1-1B.2, for example one or more of Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/ruminococcaceae, or a combination of two or more of these. In some embodiments, the first binding agent is specific to Bacteroides ovatus, Parabacteroides merdae, Bacteroides thetaiotaomicron, Eisenbergiela tayi, or a combination of two or more of these. In some embodiments, the first binding agent is also joined to a first support. In some embodiments, the first support is a plastic, such as a polystyrene or polyvinylchloride, a chip, a membrane, such as a nylon or nitrocellulose membrane, a lateral flow device, a bead, such as an agarose, latex, acrylamide, magnetic, or polymeric bead, a fiber, such as a hollow fiber, or a filter, such as a hollow filter. In some embodiments, the first binding agent further comprises a detectable moiety. In some embodiments, the detectable moiety is selected from the group consisting of an affinity tag, a bead, a microparticle, a colored bead, a photoreactive group, a radionuclide, a hapten, a peptide, an enzyme, a fluorescent species, a luminescent species, a dye, biotin, a triazole, an alkyne, quantum dots, and a chelating species. In some embodiments, the first support further comprises a control zone comprising isolated binding target from one or more of bacteria of Tables 1A.1-1A.2 and/or 1B.1-1B.2, for example bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts. In some embodiments, the first support further comprises a control zone having an immobilized antigen from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts. In some embodiments, the composition further comprises a second binding agent joined to said antigen at a site that is distinct from a site to which said first binding agent binds said antigen. In some embodiments, the second binding agent is an antibody, such as a monoclonal antibody, or a binding fragment thereof, or an aptamer, such as a DNA aptamer. In some embodiments, the second binding agent is also joined to a second support. In some embodiments, the second support is a plastic, such as a polystyrene or polyvinylchloride, a chip, a membrane, such as a nylon or nitrocellulose membrane, a lateral flow device, a bead, such as an agarose, latex, acrylamide, magnetic, or polymeric bead, a fiber, such as a hollow fiber, or a filter, such as a hollow filter. In some embodiments, the second binding agent further comprises a detectable moiety, or wherein said first binding agent comprises a detectable moiety. In some embodiments, the detectable moiety is selected from the group consisting of an affinity tag, a bead, a microparticle, a colored bead, a photoreactive group, a radionuclide, a hapten, a peptide, an enzyme, a fluorescent species, a luminescent species, a dye, biotin, a triazole, an alkyne, quantum dots, and a chelating species. In some embodiments, the second support further comprises a control zone having an immobilized first binding agent from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts. In some embodiments, the second support further comprises a control zone having an immobilized first binding agent from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts.

In some embodiments, a method for binding an ASD-specific biomarker to a support is described. The method can comprise (a) contacting a first support that comprises a first binding agent, which specifically binds an immobilized binding target from one or more of bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae and/or parabacteroides, and/or eisenbergiela, with a biological sample that comprises a binding target from one or more bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae parabacteroides, and/or eisenbergiela. The method can comprise (b) identifying the presence or absence of the binding target bound to the support. In some embodiments, the immobilized binding target comprises a second nucleic acid, and the first binding agent comprises a first nucleic acid complementary to the second nucleic acid. In some embodiments, the second nucleic acid is specific a bacteria of Tables 1A.1-1A.2 and/or Table 1B.1-1B.2, for example one or more of Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/ruminococcaceae, or a combination of two or more of these. In some embodiments, the second nucleic acid is specific to Bacteroides ovatus, Parabacteroides merdae, Bacteroides thetaiotaomicron, Eisenbergiela tayi, or a combination of two or more of these. In some embodiments, the binding agents of the support consist essentially of a first nucleic acid which specifically binds to second nucleic acid of one or more of bacteria of bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae and/or parabacteroides, and/or eisenbergiela. In some embodiments, the binding agents of the support consists essentially of a first nucleic acid which specifically binds to Bacteroides ovatus, Parabacteroides merdae, and Bacteroides thetaiotaomicron, Eisenbergiela tayi, or a combination of two or more of these. In some embodiments, the second nucleic acid comprises a 16S RNA. In some embodiments, (a) the second nucleic acid is specific to Bacteroides ovatus, and comprises an sOTU sequence of sOTU b20cd_Bacteroides; or (b) the second nucleic acid is specific to Parabacteroides merdae, and comprises an sOTU sequence of sOTU 4ae7e_Parabacteroides; or (c) the second nucleic acid is specific to Eisenbergiela tayi, and comprises an sOTU sequence of sOTU 02b40_Lacnospiraceae and/or 29857_Lacnospiraceae; or (a) and (b); or (a) and (c); or (b) and (c); or (a), (b), and (c). In some embodiments, the second nucleic acid comprises one or more of SEQ ID NOs: 1-20, or at least 10 consecutive nucleotides thereof, for example at least 10, 15, 20, 25, 30, 35, 40, 45, or 50 nucleotides, including ranges between any two of the listed values, for example 10-50, 10-40, 10-30, 10-20, 20-50, 20-40, 20-30, 30-50, 30-40, or 40-50. In some embodiments, an initial step comprises isolating or purifying one or more binding targets from one or more of bacteria bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae. In some embodiments, the isolated binding target comprises an antigen, and the first binding agent comprises an antibody, protein, peptide, or aptamer. In some embodiments, the first binding agent is an antibody, such as a monoclonal antibody, or a binding fragment thereof, or an aptamer, such as a DNA aptamer. In some embodiments, the first support is a plastic, such as a polystyrene or polyvinylchloride, a chip, a membrane, such as a nylon or nitrocellulose membrane, a lateral flow device, a bead, such as an agarose, latex, acrylamide, magnetic, or polymeric bead, a fiber, such as a hollow fiber, or a filter, such as a hollow filter. In some embodiments, the first binding agent further comprises a detectable moiety. In some embodiments, the detectable moiety is selected from the group consisting of an affinity tag, a bead, a microparticle, a colored bead, a photoreactive group, a radionuclide, a hapten, a peptide, an enzyme, a fluorescent species, a luminescent species, a dye, biotin, a triazole, an alkyne, quantum dots, and a chelating species. In some embodiments, the first support further comprises a control zone having an immobilized binding target from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 pg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts. In some embodiments, the first support further comprises a control zone having an immobilized binding target from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts. In some embodiments, the method further comprises contacting the antigen with a second binding agent, which specifically binds to said antigen at a site distinct from the site to which the first binding agent binds said antigen. In some embodiments, the second binding agent is an antibody, such as a monoclonal antibody, or a binding fragment thereof or an aptamer, such as a DNA aptamer. In some embodiments, the second binding agent is also joined to a second support or a detection moiety. In some embodiments, the second support is a plastic, such as a plastic plate or dish, a chip, a membrane, such as a nylon or nitrocellulose membrane, a lateral flow device, a bead, such as an agarose, latex, acrylamide, magnetic, or polymeric bead, a fiber, such as a hollow fiber, or a filter, such as a hollow filter or detection molecule, such as fluorescent dye, quantum dots, enzyme etc. In some embodiments, the second binding agent further comprises a detectable moiety. In some embodiments, the detectable moiety is selected from the group consisting of an affinity tag, a bead, a microparticle, a colored bead, a photoreactive group, a radionuclide, a hapten, a peptide, an enzyme, a fluorescent species, a luminescent species, a dye, biotin, a triazole, an alkyne, quantum dots, and a chelating species. In some embodiments, the second support further comprises a control zone having an immobilized antigen from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts. In some embodiments, the second support further comprises a control zone having an immobilized antigen from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts. In some embodiments, the biological sample is obtained from a subject who has or is at risk of having autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, and/or pure autonomic failure, or any combination thereof. In some embodiments, the biological sample is selected from the group consisting of: whole blood, plasma, serum, urine, cerebrospinal fluid, saliva, lymph, aqueous humor, vitreous humor, cochlear fluid, feces, biopsy tissue, fecal biopsy, intestinal biopsy, and tears. In some embodiments, the method further comprises isolating or analyzing a nucleic acid or protein from said sample for a disease marker. In some embodiments, the method further comprises repeating at least steps (a) and (b) at a time point after initially performing at least steps (a) and (b). In some embodiments the method further comprises providing a subject from which said biological sample was obtained with a therapeutic agent and repeating at least steps (a) and (b) at a time point after providing said therapeutic agent.

In some embodiments, a method for analyzing an antigen from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae. The method can comprise, in order: (a) contacting a first support that comprises a first binding agent, which specifically binds an antigen from one or more bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, with a biological sample that comprises an antigen from one or more of bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae so as to generate a biological complex comprising the first binding agent joined to said antigen. The method can further comprise, in order, (b) contacting the biological complex that comprises the first binding agent joined to said antigen, with a second binding agent, which specifically binds to said antigen at a site distinct from the site to which said first binding agent binds said antigen. The method can further comprise, in order, (c) identifying the presence of the second binding agent joined to the antigen.

In some embodiments, a diagnostic device is described. The device can comprise a substrate, a sample reservoir in contact with the substrate, and a conjugate zone in contact with said substrate and proximal to the sample reservoir, wherein said conjugate zone is in a flow path of analytes present in a biological sample, when an amount of said biological sample is provided to the sample reservoir. The device can further comprise an amount of one or more mobilizable labeled antibodies or a binding fragment thereof specific for one or more antigens from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, wherein said one or more mobilizable labeled antibodies or binding fragments thereof are provided at the conjugate zone. The device can further comprise a capture zone in contact with the substrate, proximal to said conjugate zone but distal from said sample reservoir, wherein said capture zone is in a flow path of analytes present in the biological sample, when an amount of the biological sample is provided to said sample reservoir such that the analytes present in the biological sample contact the conjugate zone prior to contacting the capture zone, and in which the capture zone comprises a test zone and one or more control/standard zones. The device can further comprise a binding agent immobilized to the substrate at the test zone. The device can further comprise a first amount of one or more antigens from one or more of bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof immobilized to the substrate at a first control/standard zone. The first amount of one or more antigens from the one or more bacteria of Tables 1A.1-1A.2 and/or Table 1B.1-1B.2 (for example, bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof) in the first control/standard zone can be an amount of protein detectable by the mobilizable labeled antibodies or binding fragment thereof from a biological sample obtained from a healthy subject; and/or the device can further comprise a second amount of one or more antigens from one or more bacteria of Tables 1A.1-1A.2 and/or Table 1B.1-1B.2 (for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof) in the second control/standard zone is an amount of antigen detectable by said mobilizable labeled antibodies or binding fragments thereof from a biological sample obtained from a subject that has or is at risk of having a condition autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and claimally, in which the first and/or second amounts of one or more antigens from one or more bacteria of Tables 1A.1-1A.2 and/or Table 1B.1-1B.2 (for example bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof) is the detectable amount of said proteins in the same volume of the same biological sample from said healthy subject and said subject having a condition, respectively, as the biological sample being analyzed.

In some embodiments, a diagnostic device is described. The diagnostic device can comprise a substrate and a sample reservoir in contact with the substrate, in which the sample reservoir is configured to receive a biological sample from a tested subject and, wherein said sample reservoir comprises an amount of one or more mobilizable labeled antibodies or binding fragments thereof specific for one or more antigens from one or more bacteria of Tables 1A.1-1A.2 and/or Table 1B.1-1B.2 (for example, bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof). The diagnostic device can further comprise a capture zone in fluid communication with said substrate and sample reservoir, such as by capillary flow, in which the capture zone comprises a test zone and one or more control/standard zones. The diagnostic device can further comprise a binding agent immobilized to said substrate at the test zone. The diagnostic device can further comprise a first amount of one or more antigens from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof immobilized to said substrate at a first control/standard zone, wherein the first amount of protein in the first control/standard zone is an amount of protein detectable by said mobilizable labeled antibodies or binding fragments thereof in a biological sample obtained from a healthy subject. The diagnostic device can further comprise a second amount of one or more antigens from one or more bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela or any combination thereof immobilized to said substrate at a second control/standard zone, wherein the second amount of antigen in the second control/standard zone is an amount of antigen detectable by said mobilizable labeled antibodies or binding fragments thereof in a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and claimally, in which the first and/or second amounts of one or more antigens from one or more bacteria of Tables 1A.1-1A.2 and/or Table 1B.1-1B.2 (for example, bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof) is the detectable amount of said proteins in the same volume of the same biological sample from said healthy subject and said subject that has a condition, respectively, as the biological sample from said tested subject.

Some embodiments include a diagnostic device comprising a processing chamber configured to receive a first amount of a biological sample from a subject; a first porous membrane located within the processing chamber. The diagnostic device can further comprise a second porous membrane in fluid communication, such as by capillary flow, with said first porous membrane, in which the second porous membrane comprises an amount of mobilizable labeled antibodies or binding fragments thereof disposed thereon, said mobilizable labeled antibodies or binding fragments thereof being specific for one or more antigens from one or more antigens from one or more bacteria of Tables 1A.1-1A.2 and/or Table 1B.1-1B.2 (for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof). The diagnostic device can further comprise a third porous membrane in fluid communication, such as by capillary flow, with said second porous membrane, wherein said third porous membrane comprises a capture zone comprising one or more binding agents immobilized thereon. The diagnostic device can further comprise a fourth porous membrane in fluid communication, such as by capillary flow, with said second porous membrane, wherein said fourth porous membrane comprises a first quantity of one or more antigens from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof, immobilized thereon, wherein the first quantity of antigen is the amount of antigen detectable by said mobilizable labeled antibodies or binding fragments thereof in an amount of a biological sample obtained from a healthy subject; and/or the device can further comprise a fifth porous membrane in fluid communication, such as by capillary flow, with said second porous membrane, wherein said fifth porous membrane comprises a second quantity of one or more antigens from one or more bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized thereon, wherein the second quantity of antigen is the amount of antigen detectable by said mobilizable labeled antibodies or binding fragments thereof in an amount of a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and claimally, wherein the first and/or second amounts of one or more antigens from one or more antigens from one or more of bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof, is the detectable amount of said antigens in the same volume of the same biological sample from said healthy subject and said subject that has a condition, respectively, as the biological sample from said tested subject. The diagnostic device can further comprise a viewing window, in which the viewing window permits visual inspection of the third, fourth, and/or fifth porous membranes, such that the amount of one or more antigens from one or more antigens from one or more of bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, captured and detected by said mobilizable labeled antibodies at the third porous membrane can be visually compared to the quantity of one or more antigens from one or more bacteria of Tables 1A.1-1A.2 and/or Table 1B.1-1B.2 (for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof) detected by said mobilizable labeled antibodies at the fourth and/or fifth porous membranes.

In some embodiments, a dipstick diagnostic device is described. The dipstick diagnostic device can comprise a substrate that comprises a conjugate zone and processing zone, in which the conjugate zone is in fluid communication, such as by capillary flow, with said processing zone and said processing zone is configured to contact a biological sample from a tested subject. The dipstick diagnostic device can further comprise an amount of one or more mobilizable labeled antibodies or binding fragments thereof specific for one or more antigens from one or more of bacteria of Tables 1A.1-1A.2 and/or Table 1B.1-1B.2 (for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof), in which one or more mobilizable labeled antibodies or binding fragments thereof are present at said conjugate zone. The dipstick diagnostic device can further comprise a capture zone in fluid communication, such as by capillary flow, with said processing zone and said conjugate zone such that analytes present in the biological sample applied to the processing zone contact the conjugate zone prior to contacting the capture zone and, wherein the capture zone comprises a test zone and one or more control/standard zones The dipstick diagnostic device can further comprise a binding agent immobilized to said substrate at said test zone. The dipstick diagnostic device can further comprise a first amount of one or more antigens from one or more bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof, immobilized to said substrate at a first control/standard zone, wherein the first amount of protein in the first control/standard zone is an amount detectable by said mobilizable labeled antibodies or binding fragments thereof in a biological sample obtained from a healthy subject; and/or the dipstick diagnostic device can further comprise a second amount of one or more antigens from one or more of bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof, immobilized to said substrate at a second control/standard zone, in which the second amount of protein in the second control/standard zone is an amount detectable by said mobilizable labeled antibodies or binding fragments thereof in a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and claimally, in which the first and/or second amounts of one or more antigens from one or more bacteria of Tables 1A.1-1A.2 and/or Table 1B.1-1B.2 (for example, bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof), is the detectable amount of said proteins in the same volume of the same biological sample from said healthy subject and said subject that has a condition, respectively, as the biological sample from said tested subject.

In some embodiments, a diagnostic device is described. The diagnostic device can comprise a substrate comprising a sample reservoir, in which the sample reservoir is configured to receive an amount of a biological sample and said sample reservoir comprises an amount of one or more mobilizable labeled antibodies or binding fragments thereof specific for one or more antigens from one or more of bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof). The diagnostic device can further comprise an absorbent material in fluid communication, such as by capillary flow, with said substrate distal from said sample reservoir. The diagnostic device can further comprise a binding agent immobilized to the substrate at a test zone, wherein said test zone is in fluid communication, such as by capillary flow, with said sample reservoir and said absorbent material. The diagnostic device can further comprise a first amount of one or more antigens from one or more bacteria of Tables 1A.1-1A.2 and/or Table 1B.1-1B.2 (for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof) immobilized to said substrate at a first control/standard zone, in which the first control/standard zone is in fluid communication, such as by capillary flow, with the sample reservoir and, wherein the first amount of protein in the first control/standard zone is an amount detectable by said mobilizable labeled antibodies or binding fragments thereof in a biological sample obtained from a healthy subject; and/or the diagnostic device can comprise a second amount of one or more antigens from one or more bacteria of Tables 1A.1-1A.2 and/or Table 1B.1-1B.2 (for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof), immobilized to the substrate at a second control/standard zone, wherein the second amount of protein in the second control/standard zone is an amount detectable by said mobilizable labeled antibodies or binding fragments thereof in a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and claimally, in which the first and/or second amounts of one or more antigens from one or more bacteria of Tables 1A.1-1A.2 and/or Table 1B.1-1B.2 (for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof) is the detectable amount of said proteins in the same volume of the same biological sample from said healthy subject and said subject that has a condition, respectively, as the biological sample from said tested subject.

In some embodiments, a diagnostic device is described. The diagnostic device can comprise a detection region comprising a substrate; and a nucleic acid binding agent. The nucleic acid binding agent can be specific for one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, in which the nucleic acid binding agent is immobilized on the substrate. In some embodiments, the nucleic acid binding agent comprises a nucleic acid selected from the group consisting of SEQ ID NO: 1-20 or at least 10 consecutive nucleotides thereof, for example at least 10, 15, 20, 25, 30, 35, 40, 45, or 50 nucleotides, including ranges between any two of the listed values, for example 10-50, 10-40, 10-30, 10-20, 20-50, 20-40, 20-30, 30-50, 30-40, or 40-50. In some embodiments, the detection region consists essentially of the substrate and the nucleic acid binding reagent specific for one or more bacteria of Tables 1A.1-1A.2 and/or Table 1B.1-1B.2 (for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela). In some embodiments, the substrate is a membrane selected from the group consisting of polysulfone, polyethersulfone, polyamide, polyimide, nitrocellulose, PVDF, nylon and cellulose acetate, or the first, second, third, fourth, or fifth porous membrane is selected from the group consisting of polysulfone, polyethersulfone, polyamide, polyimide, nitrocellulose, PVDF, nylon and cellulose acetate. In some embodiments, the biological sample is selected from the group consisting of: whole blood, plasma, serum, urine, cerebrospinal fluid, saliva, lymph, aqueous humor, vitreous humor, cochlear fluid, feces, biopsy tissue, fecal biopsy, intestinal biopsy, and tears. In some embodiments, the label on the antibodies is colloidal carbon, colloidal gold, a fluorescent label, a quantum dot, a phosphor, a bead, a microparticle, a colored particle, a bioluminescent marker, an enzyme label, a paramagnetic particle, or a colored latex particles. In some embodiments, the binding agent is an antibody or binding fragment thereof. In some embodiments, the immobilized antigen from one or more of bacteria of Tables 1A.1-1A.2 and/or Table 1B.1-1B.2, for example, bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts. In some embodiments, the immobilized antigen from one or more of the bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts.

In some embodiments, a method for identifying a subject that is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof is described. The method can comprise detecting one or more binding targets from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof in a biological sample from said subject. The absence of, or lower level than a neurotypical control of one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, parabacteroides and/or the presence of, or higher level than a neurotypical control of lactobacillales, enterococcaceae, Enterococcus, Coprococcus, eggerthela, sutterela, alcaligenaceae, rhodospirillales, clostridaceae, rhodospirillaceae, ruminococcaceae, and/or eisenbergiela, or any combination thereof, in said biological sample can indicate increased risk of developing autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof. In some embodiments, the binding targets comprise nucleic acids, such as 16S sequences. In some embodiments, detecting one or more binding targets comprises detecting a nucleic acid selected from the group consisting of SEQ ID NO: 1-20, or at least 10 consecutive nucleotides thereof for example at least 10, 15, 20, 25, 30, 35, 40, 45, or 50 nucleotides, including ranges between any two of the listed values, for example 10-50, 10-40, 10-30, 10-20, 20-50, 20-40, 20-30, 30-50, 30-40, or 40-50. In some embodiments, nucleic acids of SEQ ID NOs: 1-20 are detected, or at least 10 consecutive nucleic acids of each of SEQ ID NOs: 1-20 are detected. In some embodiments, the binding targets comprise antigens.

In some embodiments, a method for detecting the presence of one or more antigens from one or more bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof in a biological sample, is described. The method can comprise a biological sample to any of the devices described herein. The method can further comprise determining the presence or amount of one or more antigens from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof captured at said capture zone or test zone of said device.

In some embodiments, a method for identifying a subject that is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof, is described. The method can comprise applying a biological sample to any device as described herein. The method can further comprise identifying the subject as being at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and claimally, wherein when the amount of one or more antigens from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof captured at the capture zone or test zone is greater than the amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof detected in the first amount of one or more antigens from one or more bacteria of Tables 1A.1-1A.2 and/or Table 1B.1-1B.2, for example one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof immobilized on said device.

In the some embodiments, for any of the methods or device described herein, detection of said one or more antigens from one or more antigens from one or more bacteria of Tables 1A.1-1A.2 and/or Table 1B.1-1B.2, for example, one or more bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof is by fluorescence resonance energy transfer or bioluminescence resonance energy transfer.

Some embodiments include a kit comprising a collection nucleic acids, in which each nucleic acid is specific for one or more bacteria of Tables 1A.1-1A.2 and/or Table 1B.1-1B.2, for example one or more bacteria selected from the group consisting of Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, in which the collection comprises nucleic acids specific for at least two of the listed bacteria. In some embodiments, the collection of nucleic acids consists essentially of nucleic acids specific for one or more bacteria of Tables 1A.1-1A.2 and/or Table 1B.1-1B.2, for example one or more bacteria selected from the group consisting of Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela. In some embodiments, the nucleic acids comprise a nucleic acid comprising at least 10 consecutive nucleotides of at least one of SEQ ID NO: 1-20, for example at least 10, 15, 20, 25, 30, 35, 40, 45, or 50 nucleotides, including ranges between any two of the listed values, for example 10-50, 10-40, 10-30, 10-20, 20-50, 20-40, 20-30, 30-50, 30-40, or 40-50. In some embodiments, the kit comprises at least 10 consecutive nucleotides of two or more of SEQ ID NOs: 1-20, for example at least 10, 15, 20, 25, 30, 35, 40, 45, or 50 nucleotides, including ranges between any two of the listed values, for example 10-50, 10-40, 10-30, 10-20, 20-50, 20-40, 20-30, 30-50, 30-40, or 40-50. In some embodiments, the collection of nucleic acids comprises nucleic acid amplification primers. The collection of nucleic acids can further comprise probes, for example, in embodiments in which the primers are for quantitative nucleic acid amplification. In some embodiments, the collection of nucleic acids is immobilized on a substrate. In some embodiments, the collection further comprise a detectable moiety as described herein. In some embodiments, the collection of nucleic acids is comprised by a nucleic acid panel.

### BRIEF DESCRIPTION OF THE DRAWINGS

FIGS. 1A-D: Transplantation of human fecal samples from ASD patients phenocopies behavioral deficits in wild type mice according to some embodiments. Wild type (C57Bl/6) Mice were colonized at weaning, bred at 7-8 weeks of age and their adult offspring were behaviorally tested or sacrificed for tissue harvest (FIG. 1A). Alpha and Beta Diversity from amplicon-based (Deblur) analysis of the colonic bacterial community (FIGS. 1B-C). General locomotion (by distance traveled in open field testing), repetitive behavior (by marble burying), sociability (by Direct social behavior), and communication (by USV) were tested (FIG. 1D).

FIGS. 2A-E: Discriminatory bacteria between NT- and ASD-colonized mice. Specific bacteria, at different taxonomic levels, that discriminate between NT and ASD offspring mice were identified by a linear discriminant analysis based on the 16S RNA analysis according to the OTU picking scheme of Deblur (LefSe; FIGS. 2A-B) and according to a closed reference OTU picking (FIGS. 2C-D), and by a machine learning algorithm (Random Forest; FIG. 2E), in accordance with some embodiments herein.

FIG. 3: The metabolome of ASD-colonized mice differs from that of NT-colonized mice according to some embodiments. Feces and serum samples from colonized mice were analyzed by GC-MS or NMR. Significantly different metabolites between groups were identified, as presented by volcano plots (FIG. 3A). The normalized abundance of these metabolites, by mouse and donor (FIG. 3B). Pathway analysis (FIG. 3C), performed by MetaboAvalyst 3.0 shows significant differences in amino-acid metabolism and biosynthesis (in GCMS, top panel and NMR, bottom panel).

FIGS. 4A-B: Colonization with ASD and NT microbiome induces differences in cytokines and chemokines in the serum (FIG. 4A) and in the small intestine (FIG. 4B) of colonized mice according to some embodiments. Analysis done by Bioplex.

FIG. 5: Gene expression in both the prefrontal cortex and striatum (FIG. 5A), the prefrontal cortex alone (FIG. 5B), was examined by RNA-seq. Several genes showed significant differences between groups (FIG. 5C).

FIGS. 6A-B are a series of graphs showing activity and anxiety in ASD-mice according to some embodiments.

FIGS. 6C-F are a series of graphs showing ASD-colonized mice present ASD core behavioral deficits according to some embodiments.

FIGS. 7A-C are a series of graphs showing repetitive behaviors in ASD-colonized mice correlate with patient clinical data according to some embodiments.

FIGS. 8A-H are a series of graphs showing the neuroactive fecal metabolites correlate with behavioral outcomes according to some embodiments.

FIGS. 9A-B are a series of graphs showing that humanized mice cluster by donor according to some embodiments.

FIGS. 10A-10T are sequences of 16S RNA sequences of bacteria identified as differing between the gut of ASD-colonized and NT-colonized wild type germ free mice according to some embodiments.

FIG. 11A is graph showing GI symptoms in ASD according to some embodiments.

FIGS. 11B-C are a series of graphs showing that The Microbiome in ASD Differs From That of Unrelated Controls according to some embodiments.

FIGS. 12A-D are a series of graphs showing that Human Gut Microbial Communities Modulate Behavioral Phenotypes in Mice according to some embodiments.

FIG. 13 is graph illustrating differences in gene expression in the prefrontal cortex and striatum of ASD-conlonized (right) and control (left) mice according to some embodiments.

FIGS. 14A-E are a series of drawings that relate to Lachnospiraceae, Bacteroides and Parabacteroides being observed to be differentially abundant in the TD- and ASD-offspring microbiomes according to some embodiments. FIG. 14A: Volcano plot of differential abundance analysis by DESeq2. Significantly different taxa (a0.001) are colored according to their phylum, and annotated by the genus (or next available taxonomic level by GreenGenes identification). FIG. 14B: Heatmap of differentially abundant taxa by DESeq2 (α≤0.001). Features are named according to best available taxonomy by GreenGenes with a unique feature identifier. Samples are clustered by Bray-Curtis distances. FIG. 14C: Microbiome features contributing <1% to classification between TD and ASD samples by RandomForest. FIG. 14D: Relative abundance of select features in the microbiome of male offspring, colored by donor. Hypothesis testing for differences of the means were tested by a random effects analysis and p-values from a chi-square test. NASD=20, NTD=15 (4-7 mice per donor). FIG. 14E: The abundance of select feature in the offspring microbiome is correlated with behavior of males. Spearman correlation between the microbiome and mouse behavior, by donor (See FIG. 1). Benjamini-Hochberg adjusted p-values (α≤0.05) for significant correlations are noted. Color scale denotes Spearman ρ.

### DETAILED DESCRIPTION

It has been unexpectedly discovered that transplantation of ASD gut microbiomes (e.g., a fecal transplant from ASD patients) from patients with high ADOS scores and high GIS scores (severe ASD manifestation and increased and varied gastrointestinal distress) into germ free mice phenocopies major behavioral and physiological aspects of the ASD disorder. This personalized model revealed several bacteria and metabolites that correlate with amelioration or exacerbation of behavioral phenotypes in mice and humans and, which may be used as biomarkers for diagnostic devices and/or therapeutics. Described herein is a causative role for the gut microbiome in inducing behavioral deficits in otherwise wild-type (WT) mice. Germ-free WT mice colonized with gut microbiomes from pediatric ASD patients exhibited increased repetitive behavior and communication and social deficits, compared to WT mice colonized with fecal samples from typically-developing (TD) children (also referred to as NT mice), thereby phenocopying the core behavioral indications in ASD.

In accordance with methods, devices, and kits of some embodiments herein, microbial taxa and/or metabolites shown to differ between ASD and neurotypical (NT) subjects can indicate a presence, severity, and/or risk of developing ASD.

Moreover, a metabolomic analysis showed similar trends in the relative abundance of various metabolites and specifically amino-acid degradation products as found in ASD patients. While few genes were differentially expressed between mice colonized with ASD microbiomes and those colonized with TD fecal samples (NT mice), a significant number of genes were alternatively spliced. Amongst the genes found to be differentially spliced, a significant number of ASD-associated genes were present. These findings provide evidence that gut microbes drive changes in expressed isoforms in the host brain and, as a consequence, alter behavior.

The microbial and metabolic signatures in ASD-colonized mice, compared to that of NT-colonized mice (wild-type mice colonized with bacteria from neurotypical children), provide diagnostic biomarkers, which can be used to characterize, classify, and/or diagnose a subset of ASD patients (and possibly mothers during pregnancy) via analysis of biological samples (e.g., fecal, urine, and blood samples) of patients (“patients” may also be referred to herein as “subjects,” and unless expressly stated otherwise there is no implication that a patient or subject has necessarily been admitted to the care of a particular health professional or institution). Bacteria and metabolites enriched in NT-colonized mice may be used as therapeutics or dietary supplements, e.g., either as probiotics (for bacteria) or as drugs (metabolites) during gestation or later in life. Accordingly, several methods have been devised, which utilize these diagnostic biomarkers for determining or evaluating the risk to an individual of developing ASD or ASD-like disorders, as well as, an apparatus for utilizing the methods and biomarkers disclosed herein for identifying the risk of an individual developing ASD or and ASD-like disorders.

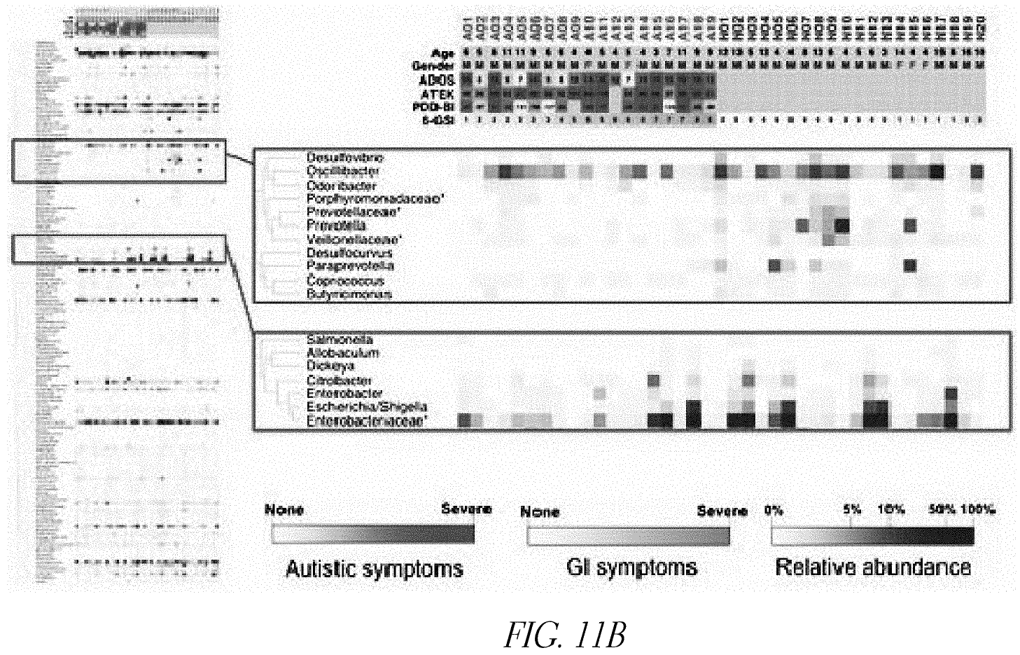

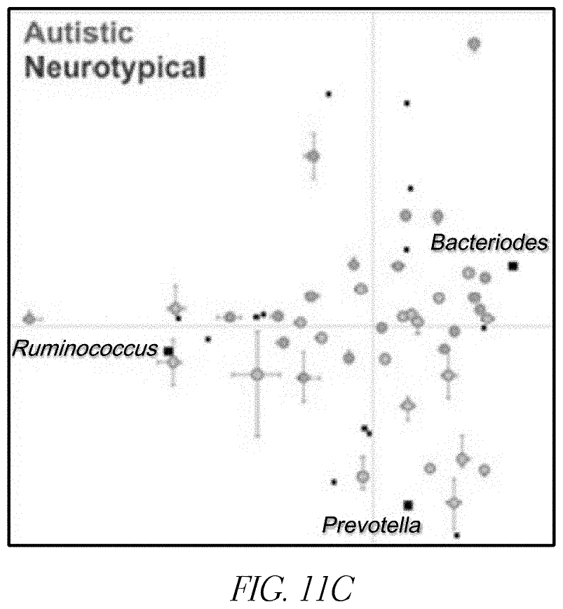

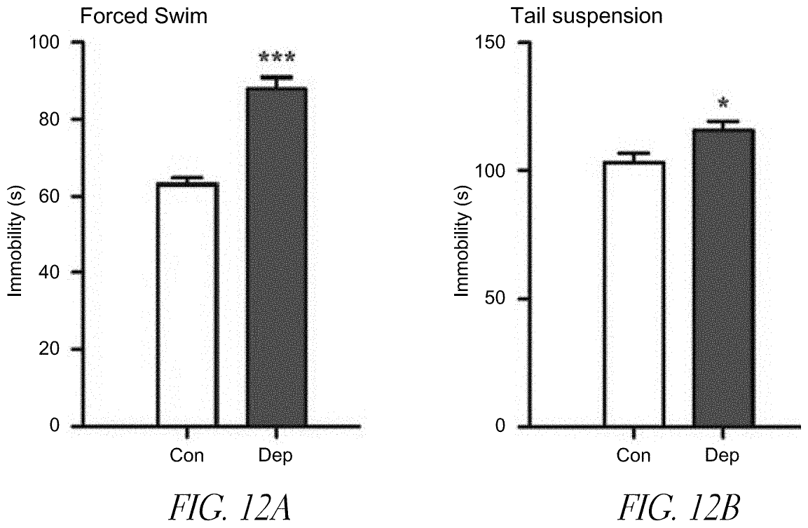

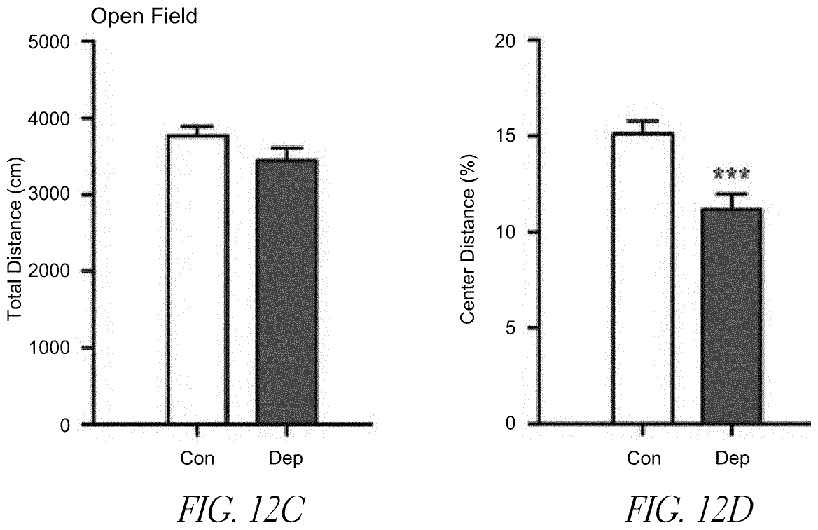

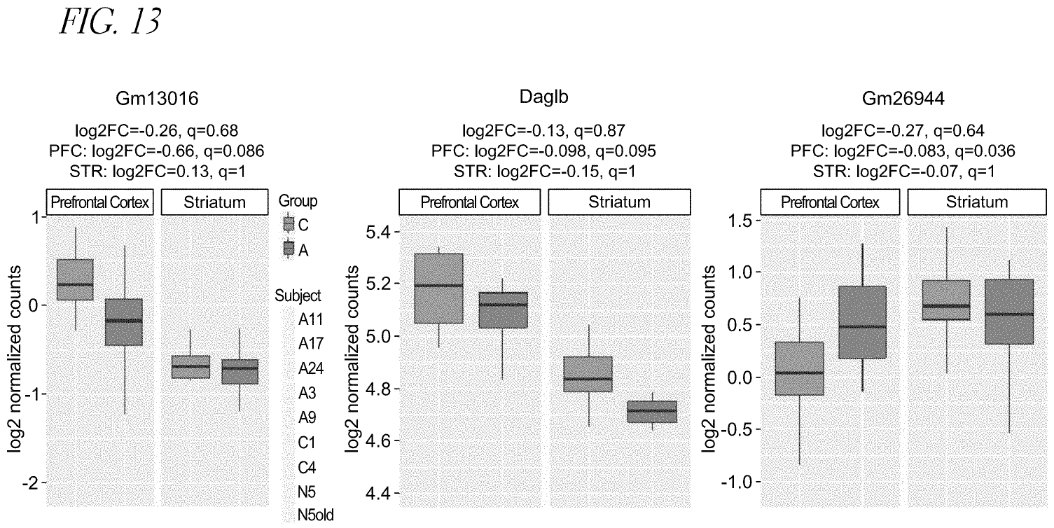

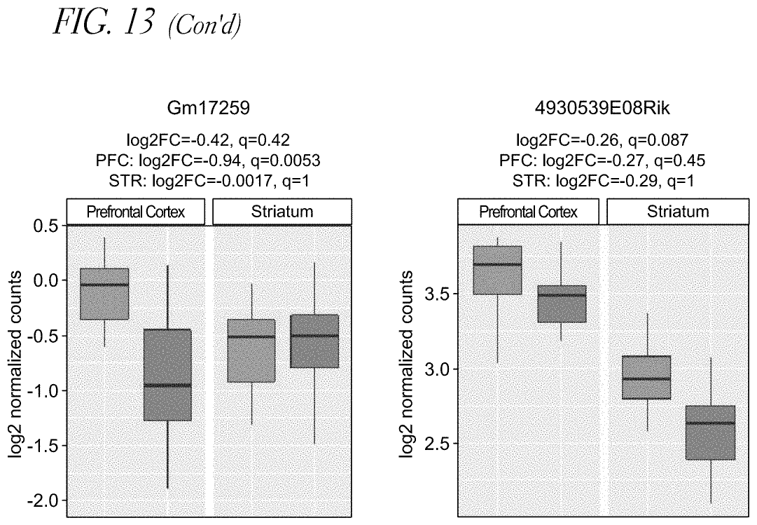

These results are consistent with other observations that GI symptoms are observed in ASD (FIG. 11A), that the guts of ASD subjects can differ from those of Neurotypical (NT) controls (FIGS. 11B-C), and that human gut microbial communities modulate behavioral phenotypes in mice (FIGS. 12A-D).

### Bacterial Taxa

It is observed herein that microbial taxa in fecal transplant samples from ASD patients that colonized germ-free WT type mice and induced behavioral indications of ASD differed from those from the fecal samples from typically-developing children that colonized germ-free WT mice that retained neurotypical behavior.

In some embodiments, the presence of bacteria of Table 1A.1 and/or Table 1A.2 in a gut sample, and/or a level of the bacteria of Table 1A.1 and/or Table 1A.2 in a gut sample that is greater than a gut sample of a neurotypical control indicate a presence, risk, or severity of ASD. It will be understood that a neurotypical control represents a comparable sample type from an individual that does not have the subject behavioral conditions or disorder, for example ASD. Neurotypical controls can be confirmed using instruments such as the ADOS. Items (a)-(d) in Table 1A.1 represent bacteria differentiated fecal samples of ASD offspring from those of NT offspring according to the OTU picking of Deblur and also according to the closed OTU picking. Item (e) in Table 1A1.1 was identified as differentiating fecal samples of ASD offspring from those of NT offspring as described in Example 2. Items (f)-(n) in Table 1A.2 were identified according to the OTU picking of Deblur or according to the closed OTU picking.

TABLE 1A.1

| | Bacteria |
|---|---|
| (a) | Lactobacillales |
| (b) | Enterococcaceae |
| (c) | Enterococcus |
| (d) | Clostridaceae |
| (e) | Eisenbergiela |

TABLE 1A.2

| | Bacteria |
|---|---|
| (f) | Coprococcus |
| (g) | Eggerthela |
| (h) | Sutterela |
| (i) | Alcaligenaceae |
| (j) | Rhodospirillales |
| (k) | Rhodospirillaceae |
| (l) | Ruminococcaceae |
| (m) | Clostridium |
| (n) | Alstipes |
| (o) | Betaproteobacteria |
| (p) | Burkholderiales |

In some embodiments, the Eisenbergiela bacteria comprises, consists essentially of, or consists of Eisenbergiela tayi.

In some embodiments, the absence of microbes of Table 1B.1 and/or Table 1B.2 in a gut sample, and/or a level of the microbes of Table 1B.1 and/or 1B.2 in a gut sample that is less than a gut sample of a neurotypical control indicate a presence or risk of ASD. Items (p)-(aa) in Table 1B.1 represent bacteria differentiated fecal samples of NT offspring from those of ASD offspring according to the OTU picking of Deblur and also according to the closed OTU picking. Items (p) and (bb) in Table 1B1.1 were identified as differentiating fecal samples NT of offspring from those of ASD offspring as described in Example 2. Items (cc)-(jj) in Table 1B.2 were identified according to the OTU picking of Deblur or according to the closed OTU picking.

TABLE 1B.1

| | |
|---|---|
| (q) | Bacteriodetes |
| (r) | Bacteroidia |
| (s) | Bacteroidales |
| (t) | Bacteroidaceae |
| (u) | Paraprevotella |
| (v) | Paraprevotellaceae |
| (w) | Rikenellaceae |
| (x) | Odoribacteraceae |
| (y) | Pseudoramibacter |
| (z) | Eubacteraciaea |
| (aa) | Holdemania |
| (bb) | Ruminococcus |
| (cc) | Parabacteroides |

TABLE 2B.2

| | |
|---|---|
| (dd) | Pseudomonadaceae |
| (ee) | Desulfobacteraceae |
| (ff) | Pseudomonadales |
| (gg) | Staphylococcus |
| (hh) | Staphylococcaceae |
| (ii) | Christensenella |
| (jj) | Anaerofilum |
| (kk) | Butyricimonas |

In some embodiments, the Bacteriodetes bacteria comprises, consists essentially of, or consists of Bacteroides ovatus and/or Bacteroides thetaiotaomicron. In some embodiments, the Parabacteroides bacteria comprises, consists essentially of, or consists of Parabacteroides merdae.

It is contemplated that in accordance with the methods, devices, and kits of some embodiments, detecting in a sample of a subject a presence (or level greater than in a sample of a neurotypical control) of any of the bacteria of Table 1A.1 and/or Table 1A.2, and/or detecting an absence (or level less than in a sample of a neurotypical control) of any of the bacteria of Table 1B.1 and/or Table 1B.2, can indicate that the subject has or is at risk of developing autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof. In some embodiments, a presence is indicated by a signal that is greater than the limit of detection (LoD). In some embodiments, a presence is indicated by a signal that exceeds a negative control, for example an assay in the absence of analyte, or an assay using a biological sample known to be neurotypical (and not have, or be at risk for, ASD). In methods, devices, and kits of some embodiments, a presence (or level greater than in a sample of a neurotypical control) of any of the bacteria of Table 1A.1 and/or Table 1A.2, and/or an absence (or level less than in a sample of a neurotypical control) of any of the bacteria of Table 1B.1 and/or Table 1B.2 is detected in a biological sample of a subject. In methods, devices, and kits of some embodiments, a presence (or level greater than in a sample of a neurotypical control) of any of the bacteria of Table 1A.1 and/or an absence (or level less than in a sample of a neurotypical control) of any of the bacteria of Table 1B.1 is detected in a biological sample of a subject. In methods, devices, and kits of some embodiments, a presence (or level greater than in a sample of a neurotypical control) of any of the bacteria of Table 1A.1 and Table 1A.2 and/or an absence (or level less than in a sample of a neurotypical control) of any of the bacteria of Table 1B.1 and Table 1B.2 is detected in a biological sample of a subject. In methods, devices, and kits of some embodiments, a presence (or level greater than in a sample of a neurotypical control) of any of the bacteria of Table 1A.2 and/or an absence (or level less than in a sample of a neurotypical control) of any of the bacteria of Table 1B.2 is detected in a biological sample of a subject. In methods, devices, and kits of some embodiments, a presence (or level greater than in a sample of a neurotypical control) of any of the bacteria of Table 1A.1 and Table 1A.2 and/or an absence (or level less than in a sample of a neurotypical control) of any of the bacteria of Table 1B.1 is detected in a biological sample of a subject. In methods, devices, and kits of some embodiments, a presence (or level greater than in a sample of a neurotypical control) of any of the bacteria of Table 1A.1 and/or an absence (or level less than in a sample of a neurotypical control) of any of the bacteria of Table 1B.1 and Table 1B.2 is detected in a biological sample of a subject.

It is further contemplated that in accordance with the methods, devices, and kits of some embodiments, detecting in a sample of a subject a presence and/or or level of two or more of the bacteria of Table 1A.1, Table 1A.2, Table 1B.1, and/or Table 1B.2 can provide superior accuracy to only a single bacteria, for example minimizing false positives. For example, the method, device, and/or kit can comprise detecting a presence and/or level at least one bacteria of Tables 1A.1-1A.2, and a presence and/or or level of at least one bacteria of Table 1B.1-1B.2. For example, the method, device, and/or kit can comprise detecting a presence and/or level at least two bacteria of Tables 1A.1-1A.2, and a presence and/or or level of at least one bacteria of Table 1B.1-1B.2. For example, the method, device, and/or kit can comprise detecting a presence and/or level at least one bacteria of Tables 1A.1-1A.2, and a presence and/or level of at least two bacteria of Table 1B.1-1B.2. In some embodiments, a composition, device, method, or kit comprises, consists essentially of, or consists of binding agents specific for one or more of any of the bacteria listed in Tables 1A.1-A.2 and/or Tables 1B.1-B.2, for example, at least 2, 3, 4, 5, 6, 7, 8, 9, 10, 11, 12, 13, 14, 15, 16, 17, 18, 19, 20, 21, 22, 23, 24, 25, 26, 27, 28 29, or 30 of the bacteria, including ranges between any two of the listed values, for example 1-5, 1-10, 1-20, 1-25, 1-30, 2-5, 2-10, 2-20, 2-25, 2-30, 3-5, 3-10, 3-20, 3-25, 3-30, 5-10, 5-20, 5-25, or 5-30.

In some embodiments, a composition, device, method, or kit comprises, consists essentially of, or consists of binding agents specific for at least two of any of the bacteria listed in Table 1A.1, for example (a) and (b), (a) and (c), (a) and (d), (a) and (e), (b) and (c), (b) and (d), (b) and (e), (c) and (d), (c) and (e), (d) and (e). In some embodiments, a composition, device, method, or kit comprises, consists essentially of, or consists of binding agents specific for (a)-(e) of Table 1A.1. In some embodiments, a composition, device, method, or kit comprises, consists essentially of, or consists of binding agents specific for at least two of any of the bacteria listed in Tables 1A.1-1A.2, for example (a) and (b), (a) and (c), (a) and (d), (a) and (e), (a), and (f), (a) and (g), (a) and (h), (a) and (i), (a) and (j), (a) and (k), (a) and (l), (a) and (m), (a) and (n), (a) and (o), (a) and (p), (b) and (c), (b) and (d), (b) and (e), (b), and (f), (b) and (g), (b) and (h), (b) and (i), (b) and (j), (b) and (k), (b) and (l), (b) and (m), (b) and (n), (b) and (o), (b) and (p), (c) and (d), (c) and (e), (c), and (f), (c) and (g), (c) and (h), (c) and (i), (c) and (j), (c) and (k), (c) and (l), (c) and (m), (c) and (n), (c) and (o), (c) and (p), (d) and (e), (d), and (f), (d) and (g), (d) and (h), (d) and (i), (d) and (j), (d) and (k), (d) and (l), (d) and (m), (d) and (n), (d) and (o), (d) and (p), (e), and (f), (e) and (g), (e) and (h), (e) and (i), (e) and (j), (e) and (k), (e) and (l), (e) and (m), (e) and (n), (e) and (o), (e) and (p), (f) and (g), (f) and (h), (f) and (i), (f) and (j), (f) and (k), (f) and (l), (f) and (m), (f) and (n), (f) and (o), (f) and (p), (g) and (h), (g) and (i), (g) and (j), (g) and (k), (g) and (l), (g) and (m), (g) and (n), (g) and (o), (g) and (p), (h) and (i), (h) and (j), (h) and (k), (h) and (l), (h) and (m), (h) and (n), (h) and (o), (h) and (p), (i) and (j), (i) and (k), (i) and (l), (i) and (m), (i) and (n), (i) and (o), (i) and (p), j) and (k), (j) and (l), (j) and (m), (j) and (n), (j) and (o), (j) and (p), (k) and (l), (k) and (m), (k) and (n), (k) and (o), (k) and (p), (l) and (m), (l) and (n), (l) and (o), (l) and (p), (m) and (n), (m) and (o), (m) and (p), (n) and (o), (n) and (p), or (o) and (p). In some embodiments, a composition, device, method, or kit comprises, consists essentially of, or consists of binding agents specific for at least two of any of the bacteria listed in Tables 1A.1-1A.2, but does not include (a), (b), (c), (d), (e), (f), (g), (h), (i), (j), (k), (l), (m), (n), (o), or (p). In some embodiments, a composition, device, method, or kit comprises, consists essentially of, or consists of binding agents specific for all of the bacteria listed in Tables 1A.1-1A.2, except for (a), (b), (c), (d), (e), (f), (g), (h), (i), (j), (k), (l), (m), (n), (o), or (p). In some embodiments, a composition, device, method, or kit comprises, consists essentially of, or consists of binding agents specific for at least two of any of the bacteria listed in Table 1A.1, but does not include (a), (b), (c), (d), or (e). In some embodiments, a composition, device, method, or kit comprises, consists essentially of, or consists of binding agents specific for all of the bacteria listed in Table 1A.1, but does not include (a), (b), (c), (d), or (e). In some embodiments, the composition, device, method, or kit comprises, consists essentially of, or consists of binding agents specific for at least 3, 4, 5, 6, 7, 8, 9, or 10 of any of the bacteria listed in Tables 1A.1-1A.2, including ranges between any two of the listed values. In some embodiments, the composition, device, method, or kit further comprises, a binding agent specific for at least one of the bacteria listed in Table 1B.1 and/or Table 1B.2.

In some embodiments, a composition, device, method, or kit comprises, consists essentially of, or consists of binding agents specific for at least two of any of the bacterial species listed in Table 1B.1, for example (q) and (r), (q) and (s), (q) and (t), (q) and (u), (q) and (v), (q) and (w), (q) and (x), (q) and (y), (q) and (z), (q) and (aa), (q) and (bb), (q) and (cc), (r) and (s), (r) and (t), (r) and (u), (r) and (v), (r) and (w), (r) and (x), (r) and (y), (r) and (z), (r) and (aa), (r) and (bb), (r) and (cc), (s) and (t), (s) and (u), (s) and (v), (s) and (w), (s) and (x), (s) and (y), (s) and (z), (s) and (aa), (s) and (bb), (s) and (cc), (t) and (u), (t) and (v), (t) and (w), (t) and (x), (t) and (y), (t) and (z), (t) and (aa), (t) and (bb), (t) and (cc), (u) and (v), (u) and (w), (u) and (x), (u) and (y), (u) and (z), (u) and (aa), (u) and (bb), (u) and (cc), (v) and (w), (v) and (x), (v) and (y), (v) and (z), (v) and (aa), (v) and (bb), (v) and (cc), (w) and (x), (w) and (y), (w) and (z), (w) and (aa), (w) and (bb), (w) and (cc), (x) and (y), (x) and (z), (x) and (aa), (x) and (bb), (x) and (cc), (y) and (z), (y) and (aa), (y) and (bb), (y) and (cc), (z) and (aa), (z) and (bb), (z) and (cc), (aa) and (bb), (aa) and (cc), or (bb) and (cc). In some embodiments, a composition, device, method, or kit comprises, consists essentially of, or consists of binding agents specific for at least two of any of the bacterial species listed in Tables 1B.1-1B.2, for example (q) and (r), (q) and (s), (q) and (t), (q) and (u), (q) and (v), (q) and (w), (q) and (x), (q) and (y), (q) and (z), (q) and (aa), (q) and (bb), (q) and (cc), (q) and (dd), (q) and (ee), (q) and (ff), (q) and (gg), (q) and (hh), (q) and (ii), (q) and (jj), (q) and (kk), (r) and (s), (r) and (t), (r) and (u), (r) and (v), (r) and (w), (r) and (x), (r) and (y), (r) and (z), (r) and (aa), (r) and (bb), (r) and (cc), (r) and (dd), (r) and (ee), (r) and (ff), (r) and (gg), (r) and (hh), (r) and (ii), (r) and (jj), (r) and (kk), (s) and (t), (s) and (u), (s) and (v), (s) and (w), (s) and (x), (s) and (y), (s) and (z), (s) and (aa), (s) and (bb), (s) and (cc), (s) and (dd), (s) and (ee), (s) and (ff), (s) and (gg), (s) and (hh), (s) and (ii), (s) and (jj), (s) and (kk), (t) and (u), (t) and (v), (t) and (w), (t) and (x), (t) and (y), (t) and (z), (t) and (aa), (t) and (bb), (t) and (cc), (t) and (dd), (t) and (ee), (t) and (ff), (t) and (gg), (t) and (hh), (t) and (ii), (t) and (jj), (t) and (kk), (u) and (v), (u) and (w), (u) and (x), (u) and (y), (u) and (z), (u) and (aa), (u) and (bb), (u) and (cc), (u) and (dd), (u) and (ee), (u) and (ff), (u) and (gg), (u) and (hh), (u) and (ii), (u) and (jj), (u) and (kk), (v) and (w), (v) and (x), (v) and (y), (v) and (z), (v) and (aa), (v) and (bb), (v) and (cc), (v) and (dd), (v) and (ee), (v) and (ff), (v) and (gg), (v) and (hh), (v) and (ii), (v) and (jj), (v) and (kk), (w) and (x), (w) and (y), (w) and (z), (w) and (aa), (w) and (bb), (w) and (cc), (w) and (dd), (w) and (ee), (w) and (ff), (w) and (gg), (w) and (hh), (w) and (ii), (w) and (jj), (w) and (kk), (x) and (y), (x) and (z), (x) and (aa), (x) and (bb), (x) and (cc), (x) and (dd), (x) and (ee), (x) and (ff), (x) and (gg), (x) and (hh), (x) and (ii), (x) and (jj), (x) and (kk), (y) and (z), (y) and (aa), (y) and (bb), (y) and (cc), (y) and (dd), (y) and (ee), (y) and (ff), (y) and (gg), (y) and (hh), (y) and (ii), (y) and (jj), (y) and (kk), (z) and (aa), (z) and (bb), (z) and (cc), (z) and (dd), (z) and (ee), (z) and (ff), (z) and (gg), (z) and (hh), (z) and (ii), (z) and (jj), (z) and (kk), (aa) and (bb), (aa) and (cc), (aa) and (dd), (aa) and (ee), (aa) and (ff), (aa) and (gg), (aa) and (hh), (aa) and (ii), (aa) and (jj), (aa) and (kk), (bb) and (cc), (bb) and (dd), (bb) and (ee), (bb) and (ff), (bb) and (gg), (bb) and (hh), (bb) and (ii), (bb) and (jj), (bb) and (kk), (cc) and (dd), (cc) and (ee), (cc) and (ff), (cc) and (gg), (cc) and (hh), (cc) and (ii), (cc) and (jj), (cc) and (kk), (dd) and (ee), (dd) and (ff), (dd) and (gg), (dd) and (hh), (dd) and (ii), (dd) and (jj), (dd) and (kk), (ee) and (ff), (ee) and (gg), (ee) and (hh), (ee) and (ii), (ee) and (jj), (ee) and (kk), (ff) and (gg), (ff) and (hh), (ff) and (ii), (ff) and (jj), (ff) and (kk), (gg) and (hh), (gg) and (ii), (gg) and (jj), (gg) and (kk), (hh) and (ii), (hh) and (jj), (hh) and (kk), (ii) and (jj), (ii) and (kk), or (q) and (jj). In some embodiments, the composition, device, method, or kit comprises, consists essentially of, or consists of binding agents specific for at least 3, 4, 5, 6, 7, 8, 9, or 10 of any of the bacteria listed in Tables 1B.1-1B.2, including ranges between any two of the listed values. In some embodiments, the composition, device, method, or kit comprises, consists essentially of, or consists of binding agents specific for at least 3, 4, 5, 6, 7, 8, 9, or 10 of any of the bacteria listed in Table 1B.1, including ranges between any two of the listed values. In some embodiments, the composition, device, method, or kit comprises, consists essentially of, or consists of binding agents specific for at least 3, 4, 5, 6, 7, 8, 9, or 10 of any of the bacteria listed in Table 1B.2, including ranges between any two of the listed values. In some embodiments, the composition, device, method, or kit comprises, consists essentially of, or consists of binding agents specific for at least 3, 4, 5, 6, 7, 8, 9, or 10 of any of the bacteria listed in Table 1B.1, including ranges between any two of the listed values, but does not include (q), (r), (s), (t), (u), (v), (w), (x), (y), (z), (aa), (bb), or (cc). In some embodiments, the composition, device, method, or kit comprises, consists essentially of, or consists of binding agents specific for at least all of the bacteria listed in Table 1B.1, except for (q), (r), (s), (t), (u), (v), (w), (x), (y), (z), (aa), (bb), or (cc). In some embodiments, the composition, device, method, or kit comprises, consists essentially of, or consists of binding agents specific for at least 3, 4, 5, 6, 7, 8, 9, or 10 of any of the bacteria listed in Table 1B.2, including ranges between any two of the listed values, but does not include (cc), (dd), (ee), (ff), (gg), (hh), (ii), (jj), or (kk). In some embodiments, the composition, device, method, or kit comprises, consists essentially of, or consists of binding agents specific for at least all of the bacteria listed in Table 1B.2, except for (cc), (dd), (ee), (ff), (gg), (hh), (ii), (jj), or (kk). In some embodiments, the composition, device, method, or kit further comprises, a binding agent specific for at least one of the bacteria listed in Table 1A.1 and/or Table 1A.2.

In some embodiments, a composition, device, method, or kit comprises, consists essentially of, or consists of binding agents specific for at least one of any of the bacteria listed in Table 1A.1 and at least one of any of the bacteria listed in Table 1B.1, for example (a) and (q), (a) and (r), (a) and (s), (a) and (t), (a) and (u), (a) and (v), (a) and (w), (a) and (x), (a) and (y), (a) and (z), (a) and (aa), (a) and (bb), (a) and (cc), (b) and (q), (b) and (r), (b) and (s), (b) and (t), (b) and (u), (b) and (v), (b) and (w), (b) and (x), (b) and (y), (b) and (z), (b) and (aa), (b) and (bb), (b) and (cc), (c) and (q), (c) and (r), (c) and (s), (c) and (t), (c) and (u), (c) and (v), (c) and (w), (c) and (x), (c) and (y), (c) and (z), (c) and (aa), (c) and (bb), (c) and (cc), (d) and (q), (d) and (r), (d) and (s), (d) and (t), (d) and (u), (d) and (v), (d) and (w), (d) and (x), (d) and (y), (d) and (z), (d) and (aa), (d) and (bb), (d) and (cc), (e) and (q), (e) and (r), (e) and (s), (e) and (t), (e) and (u), (e) and (v), (e) and (w), (e) and (x), (e) and (y), (e) and (z), (e) and (aa), (e) and (bb), or (e) and (cc). In some embodiments, the composition, device, method, or kit comprises, consists essentially of, or consists of at least one additional binding agent specific for any of the bacteria of Tables 1A.1-A.2 and/or Tables 1B.1-B.2. As such, in some embodiments, the composition, device, method, or kit comprises, consists essentially of, or consists of at least 2, 3, 4, 5, 6, 7, 8, 9, or 10 of any of the bacteria listed in Tables 1A and/or 1B, including ranges between any two of the listed values. In some embodiments, the composition, device, method, or kit comprises, consists essentially of, or consists of binding agents specific for at least one of Bacteroides ovatus, Parabacteroides merdae, Bacteroides thetaiotaomicron, and/or Eisenbergiela tayi, for example Bacteroides ovatus and Parabacteroides merdae, Bacteroides ovatus and Bacteroides thetaiotaomicron, Bacteroides ovatus and Eisenbergiela tayi, Parabacteroides merdae and Bacteroides thetaiotaomicron, Parabacteroides merdae and Eisenbergiela tayi, Bacteroides thetaiotaomicron and Eisenbergiela tayi, Bacteroides ovatus Parabacteroides merdae and Bacteroides thetaiotaomicron, Bacteroides ovatus and Parabacteroides merdae and Eisenbergiela tayi, Bacteroides ovatus and Bacteroides thetaiotaomicron and Eisenbergiela tayi, Parabacteroides merdae and Bacteroides thetaiotaomicron and Eisenbergiela tayi, or Bacteroides ovatus and Parabacteroides merdae and Bacteroides thetaiotaomicron and Eisenbergiela tayi.

Some embodiments include determining a presence and/or level in a biological sample of bacteria selected from the group consisting of: Bacteroides ovatus, Parabacteroides merdae, Bacteroides thetaiotaomicron, or a combination of two or more of the listed bacteria; and a presence and/or level of Eisenbergiela tayi. In some embodiments, a presence of E. tayi, and/or an absence of Bacteroides ovatus, Parabacteroides merdae, Bacteroides thetaiotaomicron, or a combination of two or more of the listed bacteria indicates an increased risk or presence of ASD in the subject that provided the biological sample. In some embodiments, a higher level of E. tayi, and/or a lower level of Bacteroides ovatus, Parabacteroides merdae, Bacteroides thetaiotaomicron or a combination of two or more of the listed bacteria, compared to a biological sample of a non-ASD control can indicate a presence or an elevated risk of ASD.

In some embodiments, a presence or level of any of the bacteria of Tables 1A.1-A.2 and/or Tables 1B.1-B.2 is determined by detecting a presence and/or level of an antigen specific for the indicated bacteria.

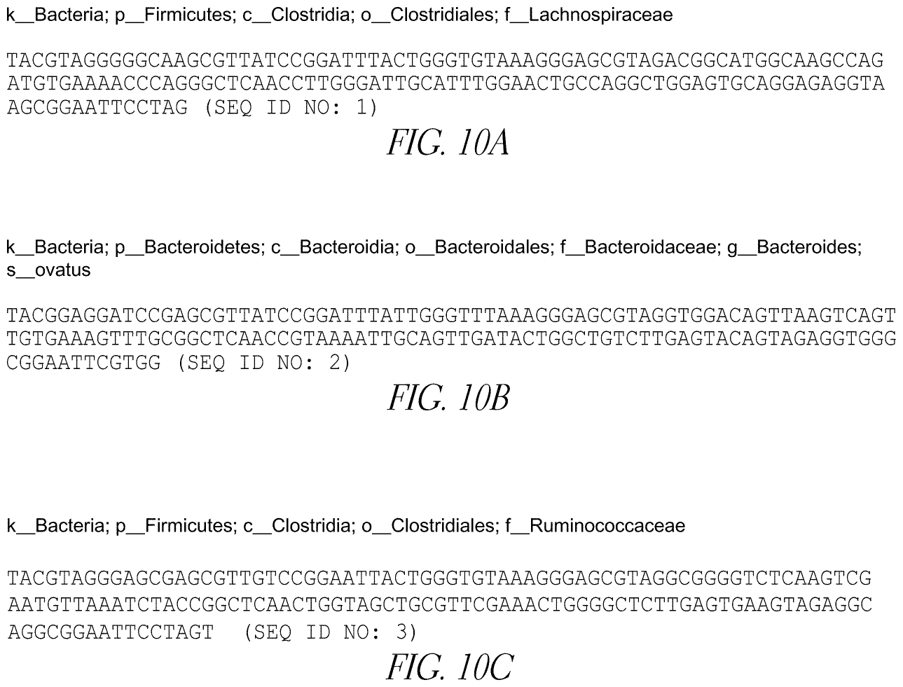

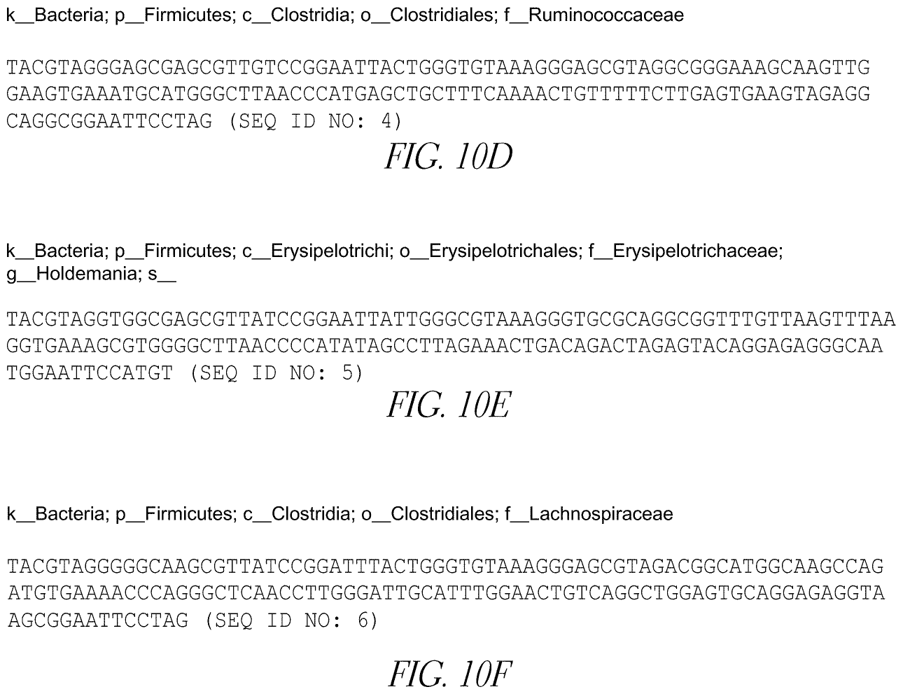

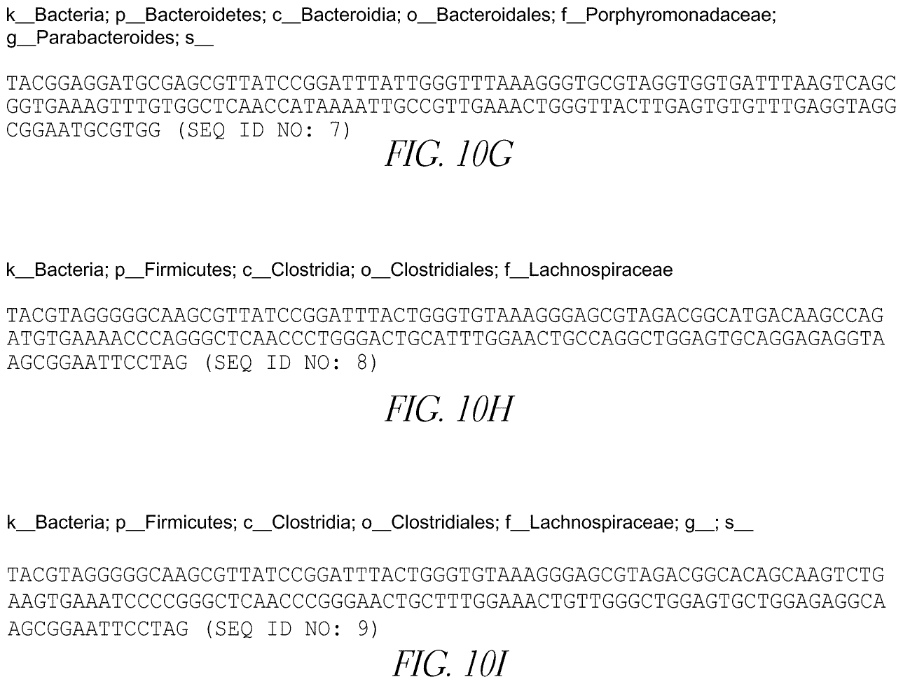

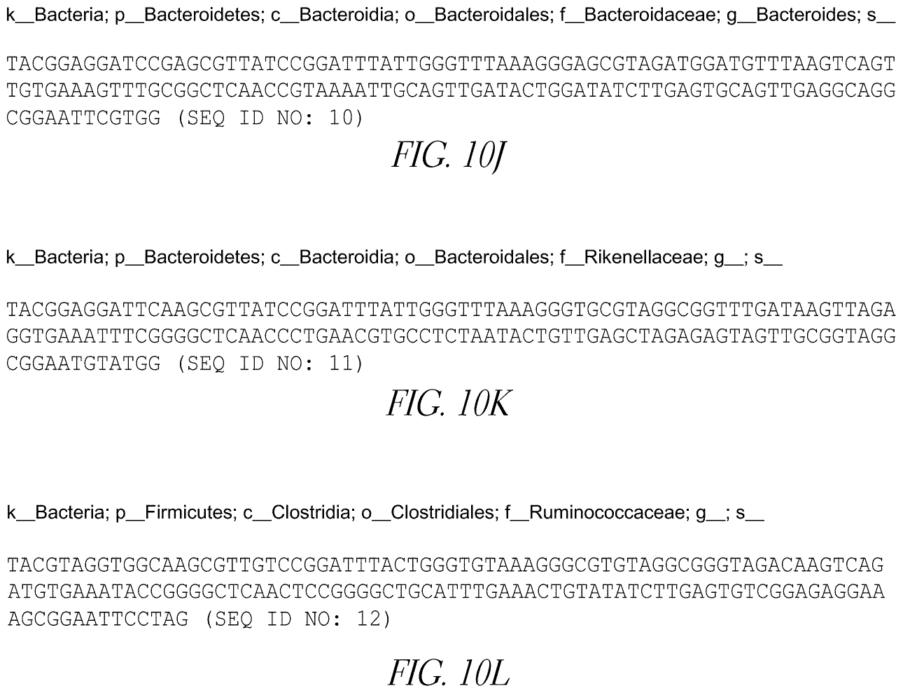

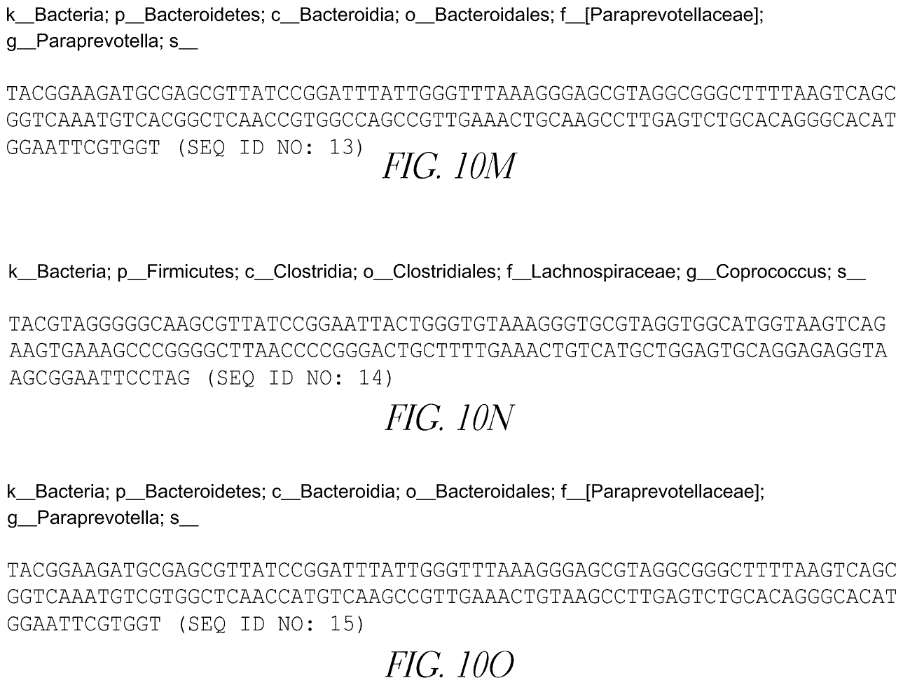

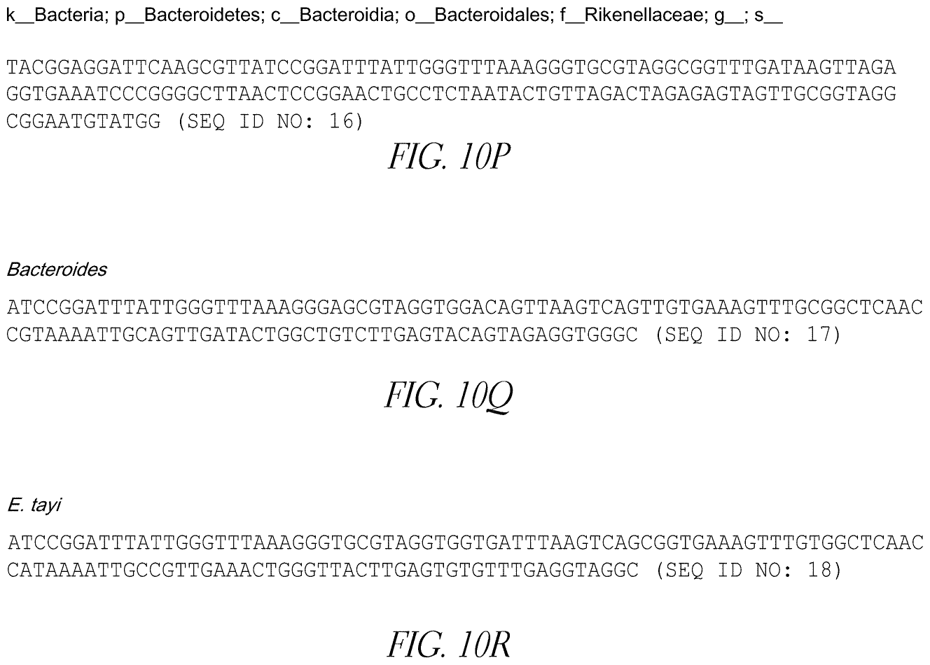

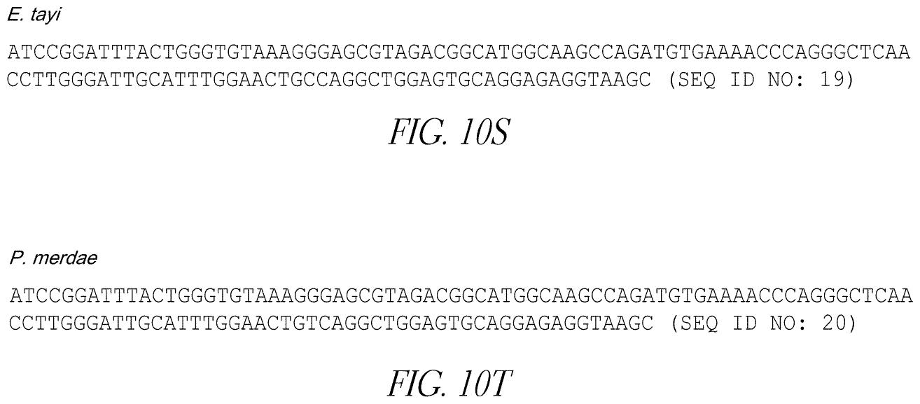

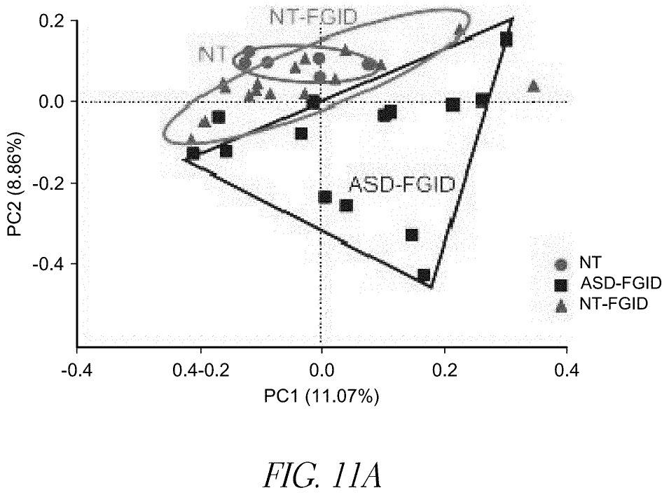

In some embodiments, a presence or level of any of the bacteria of Tables 1A.1-A.2 and/or Tables 1B.1-1B.2 is determined by detecting a presence and/or level of a nucleic acid sequence specific for the indicated bacteria, for example, the sequence for all or a portion of the gene encoding the 16S ribosomal ribonucleic acid. It is noted that wherever “16S” nucleic acids and “16S” nucleic acid sequences (including variations of these root terms) are mentioned herein, 16S RNAs, as well as DNAs encoding 16S rRNA are expressly contemplated. Example 16S sequences specific to the bacteria of Tables 1A.1-1A.2 and Tables 1B.1-1B.2 are shown in FIGS. 10A-T (SEQ ID NOs: 1-20). In some embodiments, a binding agent comprises a sequence that is complementary to at least 10 consecutive nucleotides of one or more of SEQ ID Nos: 1-20, for example at least 10, 15, 20, 25, 30, 35, 40, 45, or 50 nucleotides, including ranges between any two of the listed values, for example 10-50, 10-40, 10-30, 10-20, 20-50, 20-40, 20-30, 30-50, 30-40, or 40-50.

### Binding Agents and Binding Targets

In compositions, devices, methods, and kits of some embodiments, a “binding agent” specifically binds to a “binding target” that is specific for a bacteria of Tables 1A.1-1A.2 and/or Tables 1B.1-1B.2. Example binding agents suitable for some embodiments include antibodies and functional binding fragments thereof, and aptamers (DNA and/or peptide) which can specifically bind to antigens specific for bacteria of Tables 1A.1-1A.2 and/or Tables 1B.1-1B.2, for example proteins, carbohydrates, and/or metabolites as described herein. Example antigens can include, for example, metabolites that correlate with the presence and/or absence of the bacteria (for example, lysine, 5-aminovaleric acid, genistein, taurine, 3-aminoisobutyric acid, and/or daidzein). It will further be appreciated that a binding agent such as an antibody or fragment thereof can bind to an epitope of an antigen, and as such, the full antigen structure is not necessarily required for binding. Accordingly, unless stated otherwise, wherever a full-size “antigen” structure is mentioned herein, the relevant binding portion or epitope of the full antigen structure is also contemplated. Example binding agents suitable for some embodiments include nucleic acids which can specifically bind to nucleic acids specific for bacteria of Tables 1A.1-1A.2 and/or Tables 1B.1-1B.2, for example 16S nucleic acids. It will further be appreciated that in some embodiments, a binding agent binds to two or more nucleic acids specific for a bacteria of Tables 1A.1-1A.2 and/or Tables 1B.1-1B.2 (as it is contemplated that having multiple specific nucleic acids for a bacteria can facilitate specificity, and decrease the likelihood of false positives). Suitable nucleic acids can be found, for example, in the publicly available genome assembly of the relevant bacteria. The “binding target” as used herein, refers to the structure specifically bound by a binding agent, for example an antigen (such as a protein, carbohydrate, or metabolite) or nucleic acid. Relevant binding agent-binding target pairings will readily be appreciated in view of this disclosure, for example, antibody-antigen, aptamer-antigen, and nucleic acid-complementary nucleic acid.

It will further be appreciated that in compositions, devices, methods, and kits of some embodiments, binding agents specifically bind to their corresponding binding targets from biological samples. That is, an analyte comprising, consisting essentially of, or consisting of a biological sample or a portion thereof can contain (or be suspected of containing) binding targets for the binding agents of a method, device, or kit as described herein.

Example binding agents suitable for some methods, kits, and devices of some embodiments include nucleic acids, for example nucleic acids primers, probes, and capture sequences (which can specifically bind to nucleic acids specific for bacteria of Tables 1A.1-1A.2 and/or Tables 1B.1-1B.2, for example 16S sequences). Example 16S sequences for the bacteria of Tables 1A.1-1A.2 and/or Tables 1B.1-1B.2 are shown in FIGS. 10A-U as SEQ ID NOs: 1-20. It will readily be appreciated that in accordance with methods, kits, and devices of some embodiments, the binding agent comprises, consists essentially of or consists of a nucleic acid that is complementary to a nucleic acid sequence of SEQ NO: 1-20. In some embodiments, the nucleic acid binding agent has a length less than 200, 150, 100, 90, 80, 70, 60, 50, 40, 30, 20, or 10 nucleotides, including ranges between any two of the listed values, for example, 10-20, 10-30, 10-40, 10-50, 10-100, 10-200, 20-30, 20-40, 20-50, 20-100, 20-200, 50-100, or 50-200. It is noted that wherever “a” is mentioned herein, the plural is also contemplated, so it will be appreciated that “a” binding agent can refer to two or more binding agents. For example, “a binding agent specific for (a) and (b) of Table 1A.1 contemplates two binding agents in which one binding agent is specific for (a) and one binding agent is specific for (b), as well as a single bispecific binding agent. In some embodiments, (i) the second nucleic acid is specific to Bacteroides ovatus, and comprises an sOTU sequence of sOTU b20cd_Bacteroides, (ii) the second nucleic acid is specific to Parabacteroides merdae, and comprises an sOTU sequence of sOTU 4ae7e_Parabacteroides, (ii) the second nucleic acid is specific to Eisenbergiela tayi, and comprises an sOTU sequence of sOTU 02b40_Lachnospiraceae and/or 29857_Lachnospiraceae, (i) and (ii), (i) and (iii), (ii) and (iii), or (i), (ii), and (iii).

In some embodiments, for example, if the binding agent is an antibody (or binding fragment thereof) or probe, the binding agent further comprises a detectable moiety, for example, colloidal carbon, colloidal gold, a fluorescent label, a quantum dot, a phosphor, a bead, a microparticle, a colored particle, a bioluminescent marker, an enzyme label, a paramagnetic particle, or a colored latex particle. In some embodiments, the binding agent does not comprise a detectable moiety (for example, if analyte is labeled with a detectable moiety so as to quantify analyte bound to the binding agent).

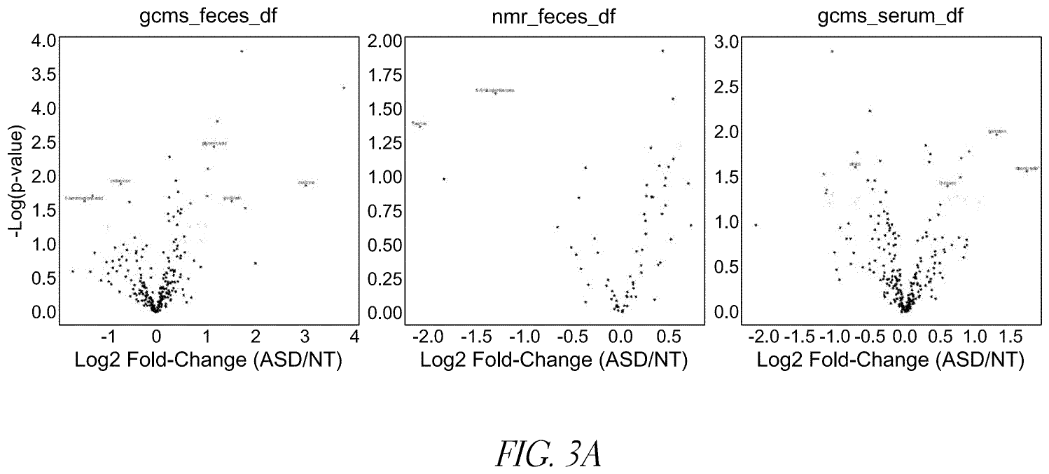

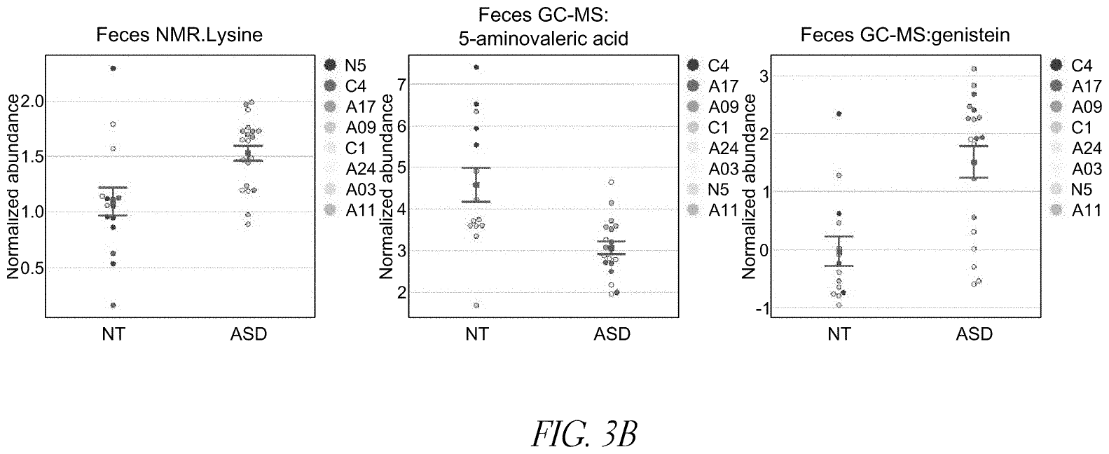

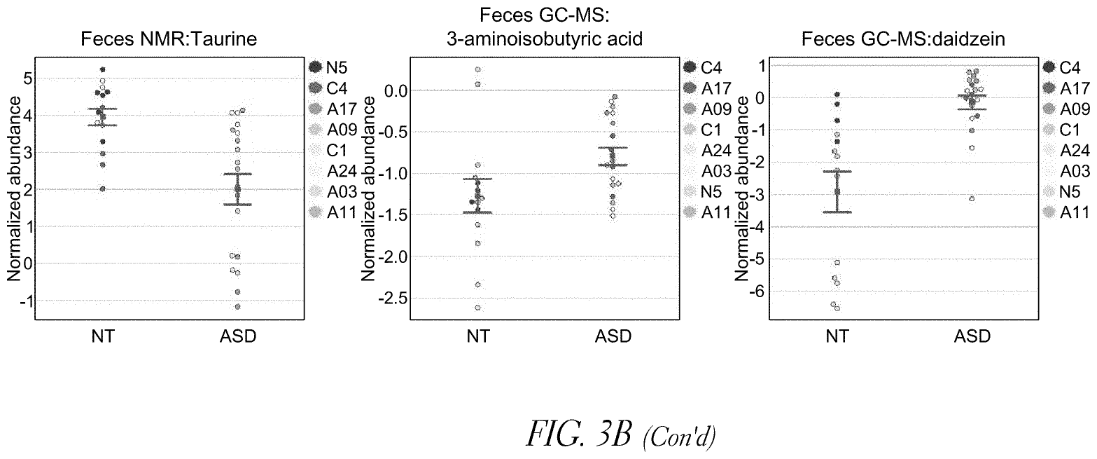

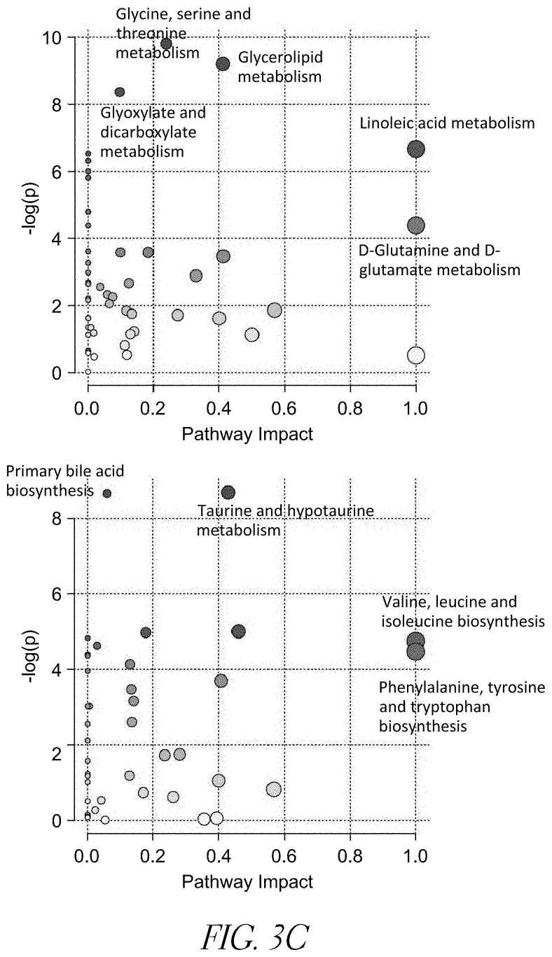

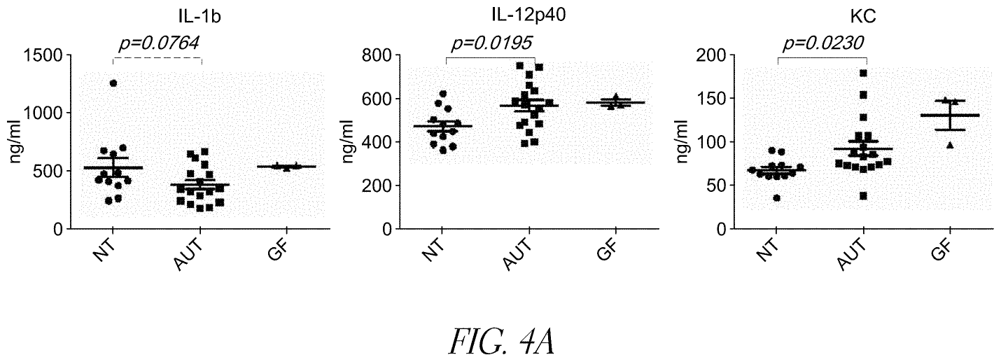

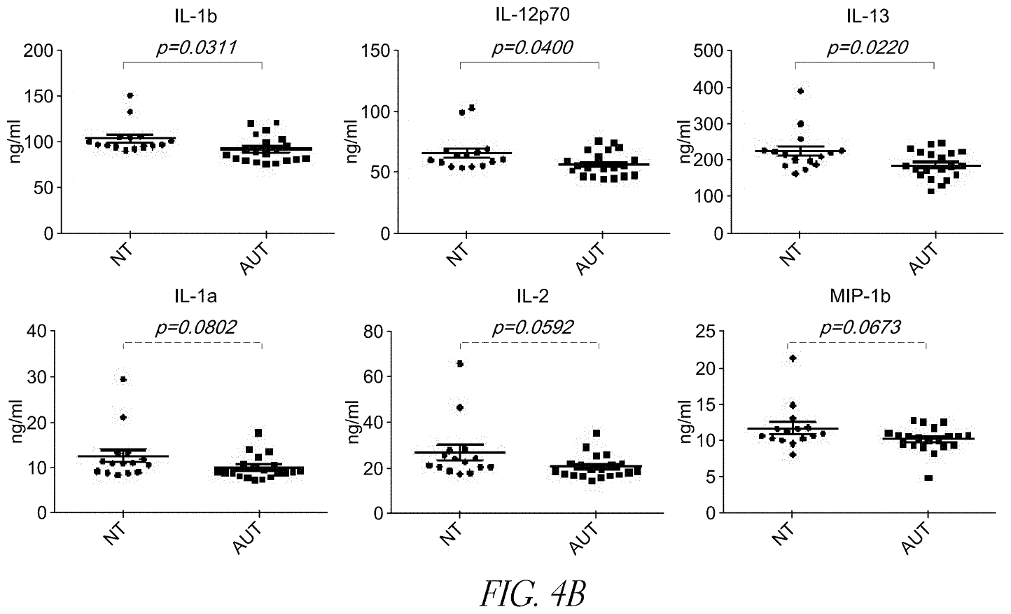

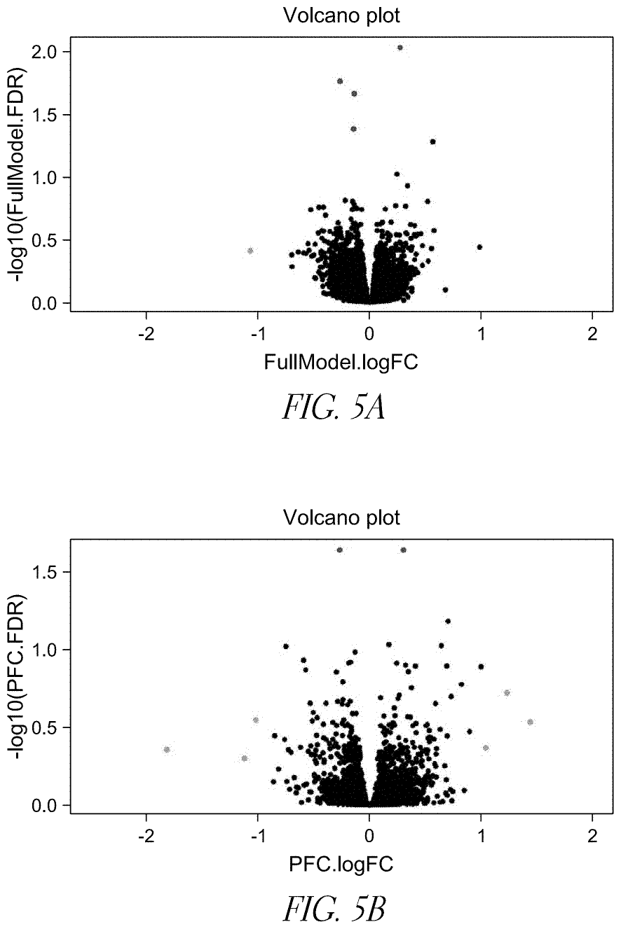

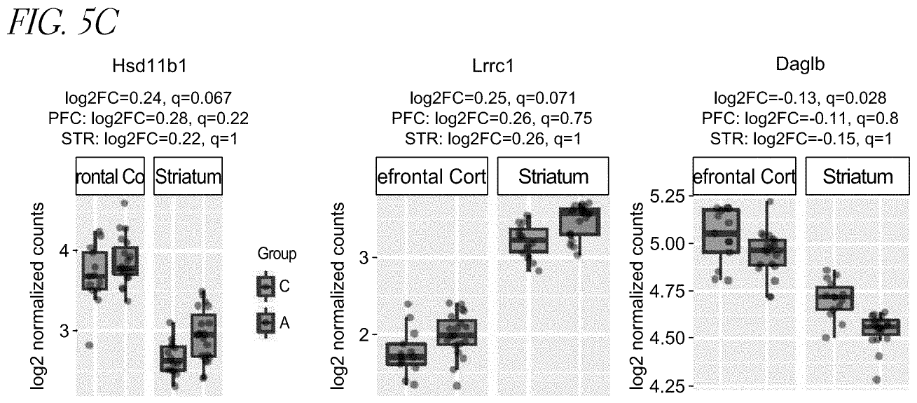

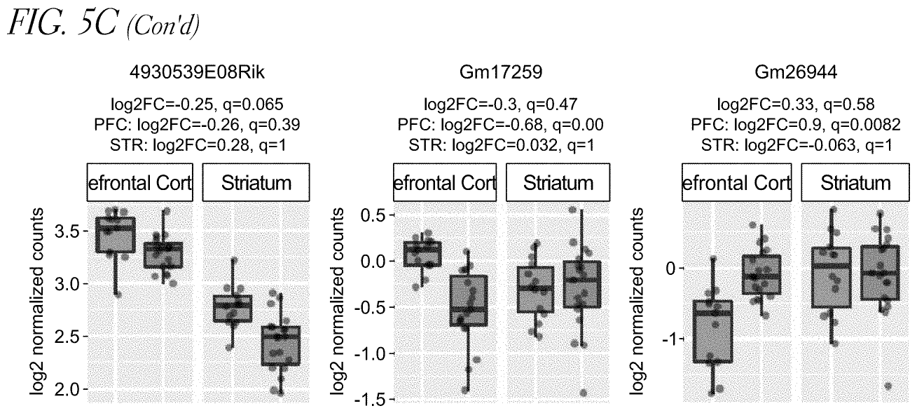

In some embodiments, the binding target is an antigen comprising, consisting essentially of, or consisting of a metabolite, for example lysine, 5-aminovaleric acid, genistein, taurine, 3-aminoisobutyric acid, and/or daidzein. It has been observed that each of these metabolites is expressed differently in ASD-colonized mice and NT-colonized control mice (FIG. 3B). In some embodiments, the antigen is an enzyme implicated in the biosynthesis of glycine, serine, and threonine; glyoxylate and dicarboxylate metabolism; glycerolipid metabolism; linoleic acid metabolism; D-glutamine and D-glutamine metabolism; primary bile acid biosynthesis; taurine and hypotaurine metabolism; valine, leucine and isoleucine biosynthesis; and/or phenylalanine, tyrosine and tryptophan biosynthesis (as shown in FIG. 3C, pathway analysis performed by MetaboAvalyst 3.0 shows significant differences in amino acid metabolism and biosynthesis via these pathways in ASD- and NT-colonized mice).

Methods, devices, and kits of some embodiments, can further comprise a second binding agent that can bind to bacteria, such as bacteria listed in Tables 1A.1-1A.2 and/or 1B.1-1B.2. In some embodiments, the second binding agent binds specifically to an antigen specific to a bacterial group listed in Tables 1A.1-1A.2 and/or 1B.1-1B.2, and binds to the antigen at a different position than the first binding agent. In some embodiments, the second binding agent binds to exopolysaccharide, pilin, lipoteichoic acid, or proteins found to be present on the exterior of bacterial cells. It is contemplated that for binding targets on (or secreted by) bacteria, such as the bacteria listed in Tables 1A.1-1A.2 and/or 1B.1-1B.2, the second binding agent can stabilize or enhance the interaction of the bacteria with the first binding agent, and/or decrease false negatives.

### Biological Samples

As used herein “biological sample” has its ordinary and customary meaning as would be understood by one of ordinary skill of the art in view of this disclosure. By way of non-limiting example, biological samples in accordance with methods, devices, and kits of some embodiments can comprise, consist essentially of, or consist of whole blood, plasma, serum, urine, cerebrospinal fluid, saliva, lymph, aqueous humor, vitreous humor, cochlear fluid, feces, biopsy tissue, fecal biopsy, intestinal biopsy, tears, or a combination of any two of the listed items.

### Devices

In accordance with some embodiments herein, devices are described. The device can be useful for detecting bacteria and/or metabolites of the gastrointestinal tract that have been shown to discriminate between ASD- and NT-subjects. The device can be used for diagnostic purposes. As such, the devices herein may be referred to as “diagnostic devices.” For example, the device can be used to diagnose one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof. In some embodiments, the device detects a presence or level of a bacteria listed in Tables 1A.1-1A.2, and/or a absence or level of a bacteria listed in Table 1B.1-1B.2. In some embodiments, the device detects a presence and/or a level of a metabolite that differs in the gastrointestinal tract of ASD- and NT-subjects, for example, a metabolite shown in FIG. 3B (e.g., lysine, 5-aminovaleric acid, genistein, taurine, 3-aminoisobutyric acid, and/or daidzein.

In some embodiments, the device is an immunoassay device, for example a lateral flow format device, and/or a dipstick device.

In some embodiments, a device comprises a substrate, a sample reservoir in contact with the substrate, and a conjugate zone in contact with said substrate and proximal to the sample reservoir. The conjugate zone is in a flow path of analytes present in a biological sample (as described herein), when an amount of said biological sample is provided to said sample reservoir. The conjugate zone can comprise one or more antibodies or binding fragments thereof specific for one or more antigens from one or more of bacteria listed in Tables 1A.1-1A.2 and/or Tables 1B.1-1B.2 (e.g., Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination of two or more of the listed bacteria). The device can further comprise a capture zone in contact with said substrate, proximal to said conjugate zone but distal from said sample reservoir. The capture zone can be in a flow path of analytes present in the biological sample when an amount of said biological sample is provided to said sample reservoir. Thus, the analytes present in the biological sample can contact the conjugate zone prior to contacting the capture zone. The capture zone can comprise a test zone and one or more control/standard zones. The device can further comprise, immobilized to the substrate at said test zone, a first amount of one or more antigens (or other binding agents) from one or more of bacteria shown in Tables 1A.1-1A.2 and/or Tables 1B.1-1B.2, for example bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof immobilized to said substrate at a first control/standard zone. The first amount of one or more antigens from one or more bacteria shown in Tables 1A.1-1A.2 and/or Tables 1B.1-1B.2, for example bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination of these in the first control/standard zone is an amount of protein detectable by said mobilizable labeled antibodies or binding fragment thereof from a biological sample obtained from a healthy subject. The device can further comprise a second amount of one or more antigens from one or more bacteria of Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela, or any combination thereof in the second control/standard zone is an amount of antigen detectable by said mobilizable labeled antibodies or binding fragments thereof from a biological sample obtained from a subject that has or is at risk of having a condition autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and optionally, wherein the first and/or second amounts of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof is the detectable amount of said proteins in the same volume of the same biological sample from said healthy subject and said subject having a condition, respectively, as the biological sample being analyzed.

Some embodiments include a diagnostic device comprising a substrate. The device can further comprise a sample reservoir in contact with the substrate, in which the sample reservoir is configured to receive a biological sample from a tested subject and, wherein said sample reservoir comprises an amount of one or more mobilizable labeled antibodies or binding fragments thereof specific for one or more antigens from one or more of bacteria listed in Table 1A and/or 1B, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof. The device can further comprise a capture zone in fluid communication with the substrate and sample reservoir, such as by capillary flow, wherein the capture zone comprises a test zone and one or more control/standard zones. The device can further comprise a binding agent immobilized to said substrate at said test zone. The device can further comprise a first amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof immobilized to the substrate at a first control/standard zone, wherein the first amount of protein in the first control/standard zone is an amount of protein detectable by said mobilizable labeled antibodies or binding fragments thereof in a biological sample obtained from a healthy subject; and/or the device can comprise a second amount of one or more antigens from one or more bacteria of Table 1A and/or 1B, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof immobilized to the substrate at a second control/standard zone, wherein the second amount of antigen in the second control/standard zone is an amount of antigen detectable by said mobilizable labeled antibodies or binding fragments thereof in a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof. In some embodiments, the first and/or second amounts of one or more antigens from one or more of bacteria listed in Tables 1A.1-1A.2 and/or Tables 1B.1-1B.2 (e.g., of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae), or any combination thereof is the detectable amount of said proteins in the same volume of the same biological sample from said healthy subject and said subject that has a condition, respectively, as the biological sample from said tested subject.

In some embodiments, the device is a diagnostic device. The device can comprise a processing chamber configured to receive a first amount of a biological sample from a subject. The device can comprise a first porous membrane located within the processing chamber. The device can comprise a second porous membrane in fluid communication, such as by capillary flow, with said first porous membrane, wherein said second porous membrane comprises an amount of mobilizable labeled antibodies or binding fragments thereof disposed thereon. The mobilizable labeled antibodies or binding fragments thereof can be specific for one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof. The device can comprise a third porous membrane in fluid communication, such as by capillary flow, with said second porous membrane, wherein said third porous membrane comprises a capture zone comprising one or more binding agents immobilized thereon. The device can comprise a fourth porous membrane in fluid communication, such as by capillary flow, with said second porous membrane, wherein said fourth porous membrane comprises a first quantity of one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized thereon, wherein the first quantity of antigen is the amount of antigen detectable by said mobilizable labeled antibodies or binding fragments thereof in an amount of a biological sample obtained from a healthy subject. The device can comprise a fifth porous membrane in fluid communication, such as by capillary flow, with said second porous membrane, wherein said fifth porous membrane comprises a second quantity of one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized thereon, wherein the second quantity of antigen is the amount of antigen detectable by said mobilizable labeled antibodies or binding fragments thereof in an amount of a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and optionally, wherein the first and/or second amounts of one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, is the detectable amount of said antigens in the same volume of the same biological sample from said healthy subject and said subject that has a condition, respectively, as the biological sample from said tested subject. The device can comprise a viewing window, wherein the viewing window permits visual inspection of the third, fourth, and/or fifth porous membranes, such that the amount of one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, captured and detected by said mobilizable labeled antibodies at the third porous membrane can be visually compared to the quantity of one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof detected by said mobilizable labeled antibodies at the fourth and/or fifth porous membranes.

In some embodiments, the device is a dipstick diagnostic device. The device can comprise a substrate that comprises a conjugate zone and processing zone, wherein said conjugate zone is in fluid communication, such as by capillary flow, with said processing zone and said processing zone is configured to contact a biological sample from a tested subject. The device can comprise one or more mobilizable labeled antibodies or binding fragments thereof specific for one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, wherein said one or more mobilizable labeled antibodies or binding fragments thereof are present at said conjugate zone. The device can comprise a capture zone in fluid communication, such as by capillary flow, with said processing zone and said conjugate zone such that analytes present in the biological sample applied to the processing zone contact the conjugate zone prior to contacting the capture zone and, wherein the capture zone comprises a test zone and one or more control/standard zones. The device can comprise a binding agent immobilized to said substrate at said test zone. The device can comprise one or more bacteria listed in Tables 1A.1-1A.2 and/or Tables 1B.1-1B.2, such as bacteria of the genus. The device can comprise a first amount of one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized to said substrate at a first control/standard zone, wherein the first amount of protein in the first control/standard zone is an amount detectable by said mobilizable labeled antibodies or binding fragments thereof in a biological sample obtained from a healthy subject; and/or a second amount of one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized to said substrate at a second control/standard zone, wherein the second amount of protein in the second control/standard zone is an amount detectable by said mobilizable labeled antibodies or binding fragments thereof in a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and optionally, wherein the first and/or second amounts of one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, is the detectable amount of said proteins in the same volume of the same biological sample from said healthy subject and said subject that has a condition, respectively, as the biological sample from said tested subject.

In some embodiments, a diagnostic device is described. The device can comprise a substrate comprising a sample reservoir, wherein said sample reservoir is configured to receive an amount of a biological sample and said sample reservoir comprises an amount of one or more mobilizable labeled antibodies or binding fragments thereof specific for one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof. The device can comprise an absorbent material in fluid communication, such as by capillary flow, with said substrate distal from said sample reservoir. The device can comprise a binding agent immobilized to said substrate at a test zone. The test zone is in fluid communication, such as by capillary flow, with said sample reservoir and the absorbent material. The device can comprise a first amount of one or more antigens one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized to said substrate at a first control/standard zone, wherein said first control/standard zone is in fluid communication, such as by capillary flow, with said sample reservoir and, wherein the first amount of protein in the first control/standard zone is an amount detectable by said mobilizable labeled antibodies or binding fragments thereof in a biological sample obtained from a healthy subject; and/or the device can comprise a second amount of one or more antigens from one or more bacteria listed in Tables 1A.1-1A.2 and/or Tables 1B.1-1B.2, such as bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized to said substrate at a second control/standard zone, wherein the second amount of protein in the second control/standard zone is an amount detectable by said mobilizable labeled antibodies or binding fragments thereof in a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and optionally, wherein the first and/or second amounts of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof is the detectable amount of said proteins in the same volume of the same biological sample from said healthy subject and said subject that has a condition, respectively, as the biological sample from said tested subject.

For any of the devices described herein, the substrate (and/or the first, second, third, fourth, and/or fifth porous membrane) can be a membrane selected from the group consisting of polysulfone, polyethersulfone, polyamide, polyimide, nitrocellulose, PVDF, nylon and cellulose acetate.

For any of the devices described herein, the biological sample can be selected from the group consisting of: whole blood, plasma, serum, urine, cerebrospinal fluid, saliva, lymph, aqueous humor, vitreous humor, cochlear fluid, feces, biopsy tissue, fecal biopsy, intestinal biopsy, and tears.

For any of the devices described herein, the label on the antibodies is colloidal carbon, colloidal gold, a fluorescent label, a quantum dot, a phosphor, a bead, a microparticle, a colored particle, a bioluminescent marker, an enzyme label, a paramagnetic particle, or a colored latex particles.

For any of the devices described herein, the binding agent is an antibody or binding fragment thereof.

In some embodiments, the immobilized antigen is from one or more bacteria listed in Tables 1A.1-1A.2 and/or Tables 1B.1-1B.2, for example a bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae. In some embodiments, the antigen is in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts.

In some embodiments, the diagnostic device comprises a substrate, and at least one immobilized nucleic acid from one or more bacteria listed in Tables 1A.1-1A.2 and/or Tables 1B.1-1B.2, for example a bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae. In some embodiments, the immobilized nucleic acid comprises at least 10 consecutive nucleotides of at least one of SEQ ID NOs: 1-20 as described herein. In some embodiments, the immobilized nucleic acid hybridizes under stringent conditions to at least one of SEQ ID NOs: 1-20. By way of example, labeled nucleotides (labeled with a detectable moiety as described herein) from a biological sample as described herein can be contacted with the diagnostic device under stringent hybridization conditions, and binding can be determined by the presence of the detectable moiety on the substrate. As used herein, “stringent hybridization conditions” has its ordinary and customary meaning as would be understood by one of ordinary skill in the art. If additional detail is desired, by way of example, stringent hybridization conditions can include prewashing in a solution of 5×SSC, 0.5% SDS, 1.0 mM EDTA (pH 8:0), and hybridizing overnight in 5×SSC at 50-65° C.

### Kits

In accordance with some embodiments, a kit is provided. The kit comprise a collection of nucleic acids, in which each nucleic acid is specific for a bacteria of Tables 1A.1-1A.2 and/or Tables 1B.1-1B.2, for example, a bacteria selected from the group consisting of Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela. The collection can comprise nucleic acids specific for at least two of the listed bacteria, for example at least 2, 3, 4, 5, 6, 7, 8, 9, 10, 11, 12, 13, 14, 15, 16, 17, 18, 19, or 20, including ranges between any two of the listed values, for example 2-20, 2-15, 2-10, 2-5, 3-20, 3-15, 3-10, 3-5, 5-20, 5-15, 5-10, 10-20, 10-15, or 15-20. In some embodiments, the collection of nucleic acids comprises nucleic acids specific for a bacterial species selected from the group consisting of Bacteroides ovatus, Parabacteroides merdae, Bacteroides thetaiotaomicron, Eisenbergiela tayi, or a combination of two or more of these. In some embodiments, the collection of nucleic acids consists essentially of nucleic acids specific for a bacteria selected from the group consisting of Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, and/or eisenbergiela. In some embodiments, the collection of nucleic acids consists essentially of nucleic acids specific for a bacteria selected from the group consisting of Bacteroides ovatus, Parabacteroides merdae, Bacteroides thetaiotaomicron, Eisenbergiela tayi, or a combination of two or more of these.

In some embodiments, the collection of nucleic acids does not comprise nucleic acids that are specific to bacteria that are not listed in Tables 1A.1-1A.2 and/or Tables 1B.1-1B.2. In some embodiments, the nucleic acids comprise a nucleic acid comprising at least 10 consecutive nucleotides of at least one of SEQ ID NO: 1-20, for example at least 10, 15, 20, 25, 30, 35, 40, 45, or 50 nucleotides, including ranges between any two of the listed values, for example 10-50, 10-40, 10-30, 10-20, 20-50, 20-40, 20-30, 30-50, 30-40, or 40-50. In some embodiments, the kit comprises at least 10 consecutive nucleotides of two or more of SEQ ID NOs: 1-20. In some embodiments, the collection of nucleic acids comprises nucleic acid amplification primer pairs. The primer pair can be for amplifying at least 10 consecutive nucleotides of at least one of SEQ ID NOs: 1-20, for example at least 10, 15, 20, 25, 30, 35, 40, 45, 50, 55, 60, 65, 70, 75, 80, 85, 90, 95, or 100 nucleotides. The primers can be suitable for nucleic acid amplification, for example qualitative or quantitative PCR, isothermal amplification, multiple displacement amplification, rolling circle amplification, and the like. Formulae for designing suitable primers are well known in the art. For example, the melting temperature (“Tm”) of a primer can be calculated based on its base composition as Tm=69.3+0.41×.(G+C) %−6−50/L, in which L is the length of the primer in nucleotides. The Tm of a hybridized primer may also be estimated using a formula adopted from hybridization assays in 1 M salt, and commonly used for calculating Tm for PCR primers: [(number of A+T)×2° C.+(number of G+C)×4° C.]. In some embodiments, a least one nucleic acid of the collection of nucleic acids hybridizes to at least one of SEQ ID NOs: 1-20 under stringent hybridization conditions. In some embodiments, each of the collection of nucleic acids hybridizes to at least one of SEQ ID NOs: 1-20 under stringent hybridization conditions. In some embodiments, the collection of nucleic acids comprises a nucleic acid panel. In some embodiments, the collection of nucleic acids is immobilized on a substrate. In some embodiments, the nucleic acids of the collection further comprise a detectable moiety as described herein. In some embodiments, the primers of the collection are in an amplification master mix, to facilitate amplification. The master mix can include the primers, buffer, dNTP's, ATP, or two or more of the listed ingredients.

### Methods

In some embodiments, methods of detecting bacteria that differentiate ASD and NT subject are provided. In some embodiments, the methods detect a presence, absence, and/or level of a bacteria and/or metabolite that is expressed differently in biological samples of ASD and NT subjects. For example, the method can detect a presence, absence, and/or level of an antigen, nucleic acid, and/or metabolite associated with ASD. In some embodiments, the method comprises determining the subject to have ASD, or an elevated risk of ASD if the biological sample of a subject exhibits an presence of at least one bacteria of Tables 1A.1-1A.2 and/or a level of at least one bacteria of Table 1A.1-1A.2 higher than a biological sample of non-ASD (e.g., NT) control, and/or if the biological sample of a subject exhibits an absence of at least one bacteria of Tables 1B.1-1B.2 and/or a level of bacteria of Tables 1B.1-1B.2 lower than a biological sample of a non-ASD (e.g., NT) control. Optionally, the method can further comprise recommending a treatment for ASD, for example administration of a therapeutic compound and/or behavior therapy. In some embodiments, the method comprises determining the subject to have ASD, or an elevated risk of ASD if the biological sample of a subject exhibits an presence of at least one bacteria of Tables 1A.1 and/or a level of at least one bacteria of Table 1A.1 higher than a biological sample of non-ASD (e.g., NT) control, and if the biological sample of a subject exhibits an absence of at least one bacteria of Tables 1B.1 and/or a level of bacteria of Tables 1B.1 lower than a biological sample of a non-ASD (e.g., NT) control. In some embodiments, the method comprises determining the subject to have ASD, or an elevated risk of ASD if the biological sample of a subject exhibits a level of at least one bacteria of Table 1A.1 higher than a biological sample of non-ASD (e.g., NT) control, and/or if the biological sample of a subject exhibits a level of bacteria of Tables 1B.1 lower than a biological sample of a non-ASD (e.g., NT) control.

Some embodiments include a method comprising receiving the result of detecting the presence, absence, and/or levels of one or more bacteria of Tables 1A.1-1A.2 and/or Tables 1B.1-1B.2 in a biological sample of a subject. The method can further comprise administering a treatment for ASD, for example administration of a therapeutic compound and/or behavior therapy, if the biological sample of the subject exhibits an presence of at least one bacteria of Tables 1A.1-1A.2 and/or a level of at least one bacteria of Tables 1A.1-1A.2 higher than a biological sample of non-ASD (e.g., NT) control, and/or if the biological sample of a subject exhibits an absence of at least one bacteria of Tables 1B.1-1B.2 and/or a level of bacteria of Tables 1B.1-1B.2 lower than a biological sample of a non-ASD (e.g., NT) control.

Some embodiments include a method for binding an ASD-specific biomarker to a support. The method can comprise contacting a first support that comprises a first binding agent, which specifically binds an immobilized binding target from one or more of bacteria listed in Tables 1A.1-1A.2 and/or Tables 1B.1-1B.2, with a biological sample that comprises a binding target from one or more bacteria listed in Tables 1A.1-1A.2 and/or Tables 1B.1-1B.2. The method can further comprise identifying the presence or absence of the binding target bound to the support. By way of example, the first binding agent can bind to an immobilized binding target from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae and/or parabacteroides, and/or eisenbergiela, and the biological sample can comprise biological sample that comprises a binding target from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae parabacteroides, and/or eisenbergiela. The method can further comprise identifying the presence or absence of the binding target bound to the support. In some embodiments, the method comprises identifying a level of binding target bound to the support. The level can be compared to a control, for example from a biological sample of a neurotypical individual.

In some embodiments, the immobilized binding target comprises a second nucleic acid, and the first binding agent comprises a first nucleic acid, which is complementary to the second nucleic acid. In some embodiments, the second nucleic acid comprises a nucleic acid sequence of at least one of SEQ ID Nos: 1-20, or at least 10 consecutive nucleotides thereof, for example at least 10, 15, 20, 25, 30, 35, 40, 45, or 50, including ranges between any two of the listed values. In some embodiments, the second nucleic acid comprises a 16S RNA. In some embodiments, the second nucleic acid is specific to at least one bacteria of Tables 1A.1-1A.2 and/or Tables 1B.1-1B.2. In some embodiments, the second nucleic acid is specific to Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/ruminococcaceae, or a combination of two or more of these. In some embodiment, the second nucleic acid is specific to Bacteroides ovatus, Parabacteroides merdae, Bacteroides thetaiotaomicron, Eisenbergiela tayi, or a combination of two or more of these. In some embodiments, the binding agents of the support consist essentially of a first nucleic acid which specifically binds to second nucleic acid of one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae and/or parabacteroides, and/or eisenbergiela. In some embodiments, (a) the second nucleic acid is specific to Bacteroides ovatus, and comprises an sOTU sequence of sOTU b20cd_Bacteroides, (b) the second nucleic acid is specific to Parabacteroides merdae, and comprises an sOTU sequence of sOTU 4ae7e_Parabacteroides, (c) the second nucleic acid is specific to Eisenbergiela tayi, and comprises an sOTU sequence of sOTU 02b40_Lacnospiraceae and/or 29857_Lacnospiraceae, (a) and (b), (a) and (c), (b) and (c), or (a), (b), and (c).

In some embodiments, an initial step of the method comprises an initial step comprises isolating or purifying one or more binding targets from one or more of bacteria listed in Table 1A and/or 1B, for example bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae.

In some embodiments, the isolated binding target comprises an antigen, and the first binding agent comprises, consists essentially of, or consists of an antibody, protein, peptide, or aptamer. In some embodiments, the first binding agent is an antibody, such as a monoclonal antibody, or a binding fragment thereof, or an aptamer, such as a DNA aptamer. In some embodiments, the first binding agent further comprises a detectable moiety, for example, an affinity tag, a bead, a microparticle, a colored bead, a photoreactive group, a radionuclide, a hapten, a peptide, an enzyme, a fluorescent species, a luminescent species, a dye, biotin, a triazole, an alkyne, quantum dots, and a chelating species, or a combination of two or more of the listed items.

In some embodiments, the first support further comprises a control zone having an immobilized binding target from one or more of bacteria listed in Tables 1A.1-1A.2 and/or Tables 1B.1-1B.2, for example bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts.

In some embodiments, the method further comprises contacting said antigen with a second binding agent, which specifically binds to said antigen at a site distinct from the site to which said first binding agent binds said antigen. For example, the second binding agent can comprise, consist essentially of, or consist of an antibody, such as a monoclonal antibody, or a binding fragment thereof or an aptamer, such as a DNA aptamer. In some embodiments, the second binding agent is also joined to a second support or a detection moiety. By way of example, the second support can comprise, consist essentially of, or consist of a plastic, such as a plastic plate or dish, a chip, a membrane, such as a nylon or nitrocellulose membrane, a lateral flow device, a bead, such as an agarose, latex, acrylamide, magnetic, or polymeric bead, a fiber, such as a hollow fiber, or a filter, such as a hollow filter or detection molecule, such as fluorescent dye, quantum dots, an enzyme, or a combination of two or more of the listed items. In some embodiments, the second binding agent further comprises a detectable moiety. By way of example, the detectable moiety can comprise, consist essentially of, or consists of an affinity tag, a bead, a microparticle, a colored bead, a photoreactive group, a radionuclide, a hapten, a peptide, an enzyme, a fluorescent species, a luminescent species, a dye, biotin, a triazole, an alkyne, quantum dots, a chelating species, or a combination of two or more of the listed items.

In some embodiments, the second support further comprises a control zone having an immobilized antigen from one or more of bacteria listed in Tables 1A.1-1A.2 and/or Tables 1B.1-1B.2, for example bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts.

In some embodiments, the biological sample is obtained from a subject who has or is at risk of having autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, and/or pure autonomic failure, or any combination thereof. In some embodiments, the biological sample comprises, consists essentially of, or consists of whole blood, plasma, serum, urine, cerebrospinal fluid, saliva, lymph, aqueous humor, vitreous humor, cochlear fluid, feces, biopsy tissue, fecal biopsy, intestinal biopsy, tears, or a combination of two or more of the listed items.

In some embodiments, the method further comprises isolating or analyzing a nucleic acid or protein from said sample for a disease marker. In some embodiments, the methods further comprising repeating the (a) contacting and (b) binding (which may also be referred to as steps (a) and (b), respectively), at a time point after initially performing at least the (a) contacting and (b) binding. In some embodiments, the method further comprises providing a subject from which the biological sample was obtained with a therapeutic agent and repeating at least steps (a) and (b) at a time point after providing the therapeutic agent.

Some embodiments include a method for analyzing an antigen from one or more of bacteria listed in Tables 1A.1-1A.2 and/or Tables 1B.1-1B.2, for example bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae. The method can comprise, in order: (a) contacting a first support that comprises a first binding agent, which specifically binds an antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, with a biological sample that comprises an antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae so as to generate a biological complex comprising the first binding agent joined to said antigen. The method can further comprise, in order, (b) contacting the biological complex that comprises the first binding agent joined to said antigen, with a second binding agent, which specifically binds to said antigen at a site distinct from the site to which said first binding agent binds said antigen. The method can further comprise, in order (c), identifying the presence of the second binding agent joined to the antigen.

In some embodiments, the first binding agent comprises or binds to exopolysaccharide, pilin, lipoteichoic acid, or proteins found to be present on the exterior of bacterial cells of one or more of the bacteria listed in Tables 1A.1-1A.2 and/or Tables 1B.1-1B.2, for example one or more of Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae. In some embodiments, the second binding agent is an agent, which specifically binds an antigen from one or more of bacteria listed in Tables 1A.1-1A.2 and/or Tables 1B.1-1B.2, for example bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae. In some embodiments, the second binding agent comprises exopolysaccharide, pilin, lipoteichoic acid, or proteins found to be present on the exterior of bacterial cells of one or more of Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae.

### Additional Options

The following options are also contemplated in accordance with some embodiments herein.

1. A composition comprising an isolated antigen joined specifically to a first binding agent, wherein said isolated antigen is from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae.

2. The composition of option 1, wherein said first binding agent is an antibody, such as a monoclonal antibody, or a binding fragment thereof, or an aptamer, such as a DNA aptamer that is specific for an isolated antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae.

3. The composition of any one of options 1-2, wherein said first binding agent is also joined to a first support.

4. The composition of option 3, wherein said first support is a plastic, such as a polystyrene or polyvinylchloride, a chip, a membrane, such as a nylon or nitrocellulose membrane, a lateral flow device, a bead, such as an agarose, latex, acrylamide, magnetic, or polymeric bead, a fiber, such as a hollow fiber, or a filter, such as a hollow filter.

5. The composition of any one of options 1-4, wherein said first binding agent further comprises a detectable moiety.

6. The composition of option 5, wherein said detectable moiety is selected from the group consisting of an affinity tag, a bead, a microparticle, a colored bead, a photoreactive group, a radionuclide, a hapten, a peptide, an enzyme, a fluorescent species, a luminescent species, a dye, biotin, a triazole, an alkyne, quantum dots, and a chelating species.

7. The composition of any one of options 1-6, wherein the first support further comprises a control zone having an immobilized antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 pg/m² to 5000 μg/m², or an amount that is within a range defined by any two amounts within one or more of the aforementioned ranges of amounts.

8. The composition of any one of options 1-7 further comprising a second binding agent joined to said antigen at a site that is distinct from a site to which said first binding agent binds said antigen.

9. The composition of option 8, wherein said second binding agent is an antibody, such as a monoclonal antibody, or a binding fragment thereof, or an aptamer, such as a DNA aptamer.

10. The composition of option 8 or 9 wherein said second binding agent is also joined to a second support.

11. The composition of option 10, wherein said second support is a plastic, such as a polystyrene or polyvinylchloride, a chip, a membrane, such as a nylon or nitrocellulose membrane, a lateral flow device, a bead, such as an agarose, latex, acrylamide, magnetic, or polymeric bead, a fiber, such as a hollow fiber, or a filter, such as a hollow filter.

12. The composition of any one of options 8-11, wherein said second binding agent further comprises a detectable moiety.

13. The composition of option 12, wherein said detectable moiety is selected from the group consisting of an affinity tag, a bead, a microparticle, a colored bead, a photoreactive group, a radionuclide, a hapten, a peptide, an enzyme, a fluorescent species, a luminescent species, a dye, biotin, a triazole, an alkyne, quantum dots, and a chelating species.

14. The composition of any one of options 8-13, wherein the second support further comprises a control zone having an immobilized antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts.

15. A method for binding an ASD-specific biomarker to a support comprising:
(a) contacting a first support that comprises a first binding agent, which specifically binds an immobilized antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, with a biological sample that comprises an antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae; and
(b) identifying the presence or absence of the antigen bound to the support.

16. The method of option 15, wherein an initial step comprises isolating or purifying one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae.

17. The method of option 15 or 16, wherein said first binding agent is an antibody, such as a monoclonal antibody, or a binding fragment thereof, or an aptamer, such as a DNA aptamer.

18. The method of any one of options 14-17, wherein said first support is a plastic, such as a polystyrene or polyvinylchloride, a chip, a membrane, such as a nylon or nitrocellulose membrane, a lateral flow device, a bead, such as an agarose, latex, acrylamide, magnetic, or polymeric bead, a fiber, such as a hollow fiber, or a filter, such as a hollow filter.

19. The method of any one of options 15-18, wherein said first binding agent further comprises a detectable moiety.

20. The method of option 19, wherein said detectable moiety is selected from the group consisting of an affinity tag, a bead, a microparticle, a colored bead, a photoreactive group, a radionuclide, a hapten, a peptide, an enzyme, a fluorescent species, a luminescent species, a dye, biotin, a triazole, an alkyne, quantum dots, and a chelating species.

21. The method of any one of options 15-20, wherein the first support further comprises a control zone having an immobilized antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts.

22. The method of any one of options 15-21, further comprising contacting said antigen with a second binding agent, which specifically binds to said antigen at a site distinct from the site to which said first binding agent binds said antigen.

23. The method of option 22, wherein said second binding agent is an antibody, such as a monoclonal antibody, or a binding fragment thereof or an aptamer, such as a DNA aptamer.

24. The method of option 22 or 23, wherein said second binding agent is also joined to a second support or a detection moiety.

25. The method of option 24, wherein said second support is a plastic, such as a plastic plate or dish, a chip, a membrane, such as a nylon or nitrocellulose membrane, a lateral flow device, a bead, such as an agarose, latex, acrylamide, magnetic, or polymeric bead, a fiber, such as a hollow fiber, or a filter, such as a hollow filter or detection molecule, such as fluorescent dye, quantum dots, enzyme etc.

26. The method of any one of options 22-25, wherein said second binding agent further comprises a detectable moiety.

27. The method of option 26, wherein said detectable moiety is selected from the group consisting of an affinity tag, a bead, a microparticle, a colored bead, a photoreactive group, a radionuclide, a hapten, a peptide, an enzyme, a fluorescent species, a luminescent species, a dye, biotin, a triazole, an alkyne, quantum dots, and a chelating species.

28. The method of any one of options 24-27, wherein the second support further comprises a control zone having an immobilized antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts.

29. The method of any one of options 15-28, wherein said biological sample is obtained from a subject who has or is at risk of having autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, and/or pure autonomic failure, or any combination thereof.

30. The method of option 29, wherein said biological sample is selected from the group consisting of: whole blood, plasma, serum, urine, cerebrospinal fluid, saliva, lymph, aqueous humor, vitreous humor, cochlear fluid, feces, biopsy tissue, fecal biopsy, intestinal biopsy, and tears.

31. The method of any one of options 15-30, further comprising isolating or analyzing a nucleic acid or protein from said sample for a disease marker.

32. The method of any one of options 15-31, further comprising repeating at least steps (a) and (b) at a time point after initially performing at least steps (a) and (b).

33. The method of any one of options 15-32, further comprising providing a subject from which said biological sample was obtained with a therapeutic agent and repeating at least steps (a) and (b) at a time point after providing said therapeutic agent.

34. A method for analyzing an antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae comprising in order:
(a) contacting a first support that comprises a first binding agent, which specifically binds an antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, with a biological sample that comprises an antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae so as to generate a biological complex comprising the first binding agent joined to said antigen;
(b) contacting the biological complex that comprises the first binding agent joined to said antigen, with a second binding agent, which specifically binds to said antigen at a site distinct from the site to which said first binding agent binds said antigen; and
(c) identifying the presence of the second binding agent joined to the antigen.

35. The method of option 34, wherein the first binding agent comprises exopolysaccharide, pilin, lipoteichoic acid, or proteins found to be present on the exterior of bacterial cells of one or more of Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae.

36. The method of any of Options 34 or 35, wherein the second binding agent is an agent, which specifically binds an antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae.

37. The method of options 8 or 22, wherein said second binding agent comprises exopolysaccharide, pilin, lipoteichoic acid, or proteins found to be present on the exterior of bacterial cells of one or more of Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae.

38. A diagnostic device comprising:

a substrate;

a sample reservoir in contact with said substrate;

a conjugate zone in contact with said substrate and proximal to said sample reservoir, wherein said conjugate zone is in a flow path of analytes present in a biological sample, when an amount of said biological sample is provided to said sample reservoir;

an amount of one or more mobilizable labeled antibodies or a binding fragment thereof specific for one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, wherein said one or more mobilizable labeled antibodies or binding fragments thereof are provided at said conjugate zone;

a capture zone in contact with said substrate, proximal to said conjugate zone but distal from said sample reservoir, wherein said capture zone is in a flow path of analytes present in the biological sample, when an amount of said biological sample is provided to said sample reservoir such that the analytes present in said biological sample contact the conjugate zone prior to contacting the capture zone, and wherein the capture zone comprises a test zone and one or more control/standard zones;

a binding agent immobilized to said substrate at said test zone;

a first amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof immobilized to said substrate at a first control/standard zone, wherein the first amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof in the first control/standard zone is an amount of protein detectable by said mobilizable labeled antibodies or binding fragment thereof from a biological sample obtained from a healthy subject, and/or

a second amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof in the second control/standard zone is an amount of antigen detectable by said mobilizable labeled antibodies or binding fragments thereof from a biological sample obtained from a subject that has or is at risk of having a condition autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and optionally, wherein the first and/or second amounts of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof is the detectable amount of said proteins in the same volume of the same biological sample from said healthy subject and said subject having a condition, respectively, as the biological sample being analyzed.

39. The device of option 38, wherein the substrate is a membrane selected from the group consisting of polysulfone, polyethersulfone, polyamide, polyimide, nitrocellulose, PVDF, nylon and cellulose acetate.

40. The device of any of Options 38-39, wherein the biological sample is selected from the group consisting of: whole blood, plasma, serum, urine, cerebrospinal fluid, saliva, lymph, aqueous humor, vitreous humor, cochlear fluid, feces, biopsy tissue, fecal biopsy, intestinal biopsy, and tears.

41. The device of any of Options 38-40, wherein the label on the antibodies or binding fragments thereof is colloidal carbon, colloidal gold, a fluorescent label, a quantum dot, a phosphor, a bead, a microparticle, a colored particle, a bioluminescent marker, an enzyme label, a paramagnetic particle, or a colored latex particles.

42. The device of any of Options 38-41, wherein the binding agent is an antibody or binding portion thereof.

43. A diagnostic device comprising:

a substrate;

a sample reservoir in contact with said substrate, wherein said sample reservoir is configured to receive a biological sample from a tested subject and, wherein said sample reservoir comprises an amount of one or more mobilizable labeled antibodies or binding fragments thereof specific for one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof;

a capture zone in fluid communication with said substrate and sample reservoir, such as by capillary flow, wherein the capture zone comprises a test zone and one or more control/standard zones;

a binding agent immobilized to said substrate at said test zone;

a first amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof immobilized to said substrate at a first control/standard zone, wherein the first amount of protein in the first control/standard zone is an amount of protein detectable by said mobilizable labeled antibodies or binding fragments thereof in a biological sample obtained from a healthy subject; and/or

a second amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof immobilized to said substrate at a second control/standard zone, wherein the second amount of antigen in the second control/standard zone is an amount of antigen detectable by said mobilizable labeled antibodies or binding fragments thereof in a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and optionally, wherein the first and/or second amounts of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof is the detectable amount of said proteins in the same volume of the same biological sample from said healthy subject and said subject that has a condition, respectively, as the biological sample from said tested subject.

44. The device of Option 43, wherein the substrate is a membrane selected from the group consisting of polysulfone, polyethersulfone, polyamide, polyimide, nitrocellulose, PVDF, nylon and cellulose acetate.

45. The device of any of Options 43-44, wherein the biological sample is selected from the group consisting of: whole blood, plasma, serum, urine, cerebrospinal fluid, saliva, lymph, aqueous humor, vitreous humor, cochlear fluid, feces, biopsy tissue, fecal biopsy, intestinal biopsy, and tears.

46. The device of any of Options 43-45, wherein the label on the antibodies is colloidal carbon, colloidal gold, a fluorescent label, a quantum dot, a phosphor, a bead, a microparticle, a colored particle, a bioluminescent marker, an enzyme label, a paramagnetic particle, or a colored latex particles.

47. The device of any of Options 43-46, wherein the binding agent is an antibody or binding fragment thereof.

48. A diagnostic device comprising:

a processing chamber configured to receive a first amount of a biological sample from a subject;

a first porous membrane located within said processing chamber;

a second porous membrane in fluid communication, such as by capillary flow, with said first porous membrane, wherein said second porous membrane comprises an amount of mobilizable labeled antibodies or binding fragments thereof disposed thereon, said mobilizable labeled antibodies or binding fragments thereof being specific for one or more antigens from one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof;

a third porous membrane in fluid communication, such as by capillary flow, with said second porous membrane, wherein said third porous membrane comprises a capture zone comprising one or more binding agents immobilized thereon;

a fourth porous membrane in fluid communication, such as by capillary flow, with said second porous membrane, wherein said fourth porous membrane comprises a first quantity of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized thereon, wherein the first quantity of antigen is the amount of antigen detectable by said mobilizable labeled antibodies or binding fragments thereof in an amount of a biological sample obtained from a healthy subject; and/or

a fifth porous membrane in fluid communication, such as by capillary flow, with said second porous membrane, wherein said fifth porous membrane comprises a second quantity of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized thereon, wherein the second quantity of antigen is the amount of antigen detectable by said mobilizable labeled antibodies or binding fragments thereof in an amount of a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and optionally, wherein the first and/or second amounts of one or more antigens from one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, is the detectable amount of said antigens in the same volume of the same biological sample from said healthy subject and said subject that has a condition, respectively, as the biological sample from said tested subject; and

a viewing window, wherein the viewing window permits visual inspection of the third, fourth, and/or fifth porous membranes, such that the amount of one or more antigens from one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, captured and detected by said mobilizable labeled antibodies at the third porous membrane can be visually compared to the quantity of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof detected by said mobilizable labeled antibodies at the fourth and/or fifth porous membranes.

49. The device of option 48, wherein the first, second, third, fourth, or fifth porous membrane is selected from the group consisting of polysulfone, polyethersulfone, polyamide, polyimide, nitrocellulose, PVDF, nylon and cellulose acetate.

50. The device of any of Options 48-49, wherein the biological sample is selected from the group consisting of: whole blood, plasma, serum, urine, cerebrospinal fluid, saliva, lymph, aqueous humor, vitreous humor, cochlear fluid, feces, biopsy tissue, fecal biopsy, intestinal biopsy, and tears.

51. The device of any of Options 48-50, wherein the label on the antibodies is colloidal carbon, colloidal gold, a fluorescent label, a quantum dot, a phosphor, a bead, a microparticle, a colored particle, a bioluminescent marker, an enzyme label, a paramagnetic particle, or a colored latex particles.

52. The device of any of Options 48-51, wherein the binding agent is an antibody or binding fragment thereof.

53. A dipstick diagnostic device comprising:

a substrate that comprises a conjugate zone and processing zone, wherein said conjugate zone is in fluid communication, such as by capillary flow, with said processing zone and said processing zone is configured to contact a biological sample from a tested subject;

an amount of one or more mobilizable labeled antibodies or binding fragments thereof specific for one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, wherein said one or more mobilizable labeled antibodies or binding fragments thereof are present at said conjugate zone;

a capture zone in fluid communication, such as by capillary flow, with said processing zone and said conjugate zone such that analytes present in the biological sample applied to the processing zone contact the conjugate zone prior to contacting the capture zone and, wherein the capture zone comprises a test zone and one or more control/standard zones;

a binding agent immobilized to said substrate at said test zone;

a first amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized to said substrate at a first control/standard zone, wherein the first amount of protein in the first control/standard zone is an amount detectable by said mobilizable labeled antibodies or binding fragments thereof in a biological sample obtained from a healthy subject; and/or

a second amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized to said substrate at a second control/standard zone, wherein the second amount of protein in the second control/standard zone is an amount detectable by said mobilizable labeled antibodies or binding fragments thereof in a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and optionally, wherein the first and/or second amounts of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, is the detectable amount of said proteins in the same volume of the same biological sample from said healthy subject and said subject that has a condition, respectively, as the biological sample from said tested subject.

54. The device of option 53, wherein the substrate is a membrane selected from the group consisting of polysulfone, polyethersulfone, polyamide, polyimide, nitrocellulose, PVDF, nylon and cellulose acetate.

55. The device of any of Options 53-54, wherein the biological sample is selected from the group consisting of: whole blood, plasma, serum, urine, cerebrospinal fluid, saliva, lymph, aqueous humor, vitreous humor, cochlear fluid, feces, biopsy tissue, fecal biopsy, intestinal biopsy, and tears.

56. The device of any of Options 53-55, wherein the label on the antibodies is colloidal carbon, colloidal gold, a fluorescent label, a quantum dot, a phosphor, a bead, a microparticle, a colored particle, a bioluminescent marker, an enzyme label, a paramagnetic particle, or a colored latex particles.

57. The device of any of Options 53-56, wherein the binding agent is an antibody or binding fragment thereof.

58. A diagnostic device, comprising:

a substrate comprising a sample reservoir, wherein said sample reservoir is configured to receive an amount of a biological sample and said sample reservoir comprises an amount of one or more mobilizable labeled antibodies or binding fragments thereof specific for one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof;

an absorbent material in fluid communication, such as by capillary flow, with said substrate distal from said sample reservoir;

a binding agent immobilized to said substrate at a test zone, wherein said test zone is in fluid communication, such as by capillary flow, with said sample reservoir and said absorbent material;

a first amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized to said substrate at a first control/standard zone, wherein said first control/standard zone is in fluid communication, such as by capillary flow, with said sample reservoir and, wherein the first amount of protein in the first control/standard zone is an amount detectable by said mobilizable labeled antibodies or binding fragments thereof in a biological sample obtained from a healthy subject; and/or

a second amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof, immobilized to said substrate at a second control/standard zone, wherein the second amount of protein in the second control/standard zone is an amount detectable by said mobilizable labeled antibodies or binding fragments thereof in a biological sample obtained from a subject that has or is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and optionally, wherein the first and/or second amounts of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof is the detectable amount of said proteins in the same volume of the same biological sample from said healthy subject and said subject that has a condition, respectively, as the biological sample from said tested subject.

59. The device of option 58, wherein the substrate is a membrane selected from the group consisting of polysulfone, polyethersulfone, polyamide, polyimide, nitrocellulose, PVDF, nylon and cellulose acetate.

60. The device of any of Options 58-59, wherein the biological sample is selected from the group consisting of: whole blood, plasma, serum, urine, cerebrospinal fluid, saliva, lymph, aqueous humor, vitreous humor, cochlear fluid, feces, biopsy tissue, fecal biopsy, intestinal biopsy, and tears.

61. The device of any of Options 58-60, wherein the binding agent is an antibody or binding fragment thereof.

62. The device of any of Options 58-61, wherein the label on the antibodies is colloidal carbon, colloidal gold, a fluorescent label, a quantum dot, a phosphor, a bead, a microparticle, a colored particle, a bioluminescent marker, an enzyme label, a paramagnetic particle, or a colored latex particles.

63. The diagnostic device of any one or more of Options 38-62, wherein the immobilized antigen from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, preferably in the amount of 1 pg/ml to 500 μg/ml, 1 pg/ml to 100 pg/ml, 100 pg/ml to 1,000 pg/ml, 1 ng/ml to 100 ng/ml, 100 ng/ml to 1,000 ng/ml, and 1 μg/ml to 500 μg/ml, or arrayed at a surface density of 10 pg/m² to 5000 μg/m², 10 pg/m² to 1000 pg/m², 1000 pg/m² to 10,000 pg/m², 10 ng/ml to 1000 ng/m², 1000 ng/m² to 10,000 ng/m², and 10 μg/m² to 5000 μg/m², or an amount that is within a range defined by any two amounts within one or more of the aforementioned ranges of amounts.

64. A method for identifying a subject that is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof, comprising detecting the presence of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof in a biological sample from said subject, wherein the presence of said one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof in said biological sample indicates increased risk of developing autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof.

65. A method for detecting the presence of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof in a biological sample comprising:

applying a biological sample to the device of any one of options 38-63; and

determining the presence or amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof captured at said capture zone or test zone of said device.

66. A method for identifying a subject that is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof, comprising:

applying a biological sample to the device of any one of options 38-63; and

identifying said subject as being at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof; and optionally, wherein when the amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof captured at the capture zone or test zone is greater than the amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof detected in the first amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof immobilized on said device.

67. The method of any of Options 15-37 or 64-66, wherein detection of said one or more antigens from one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof is by fluorescence resonance energy transfer or bioluminescence resonance energy transfer.

68. The device of any of Options 13-63, wherein detection of said one or more antigens from one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, and/or ruminococcaceae, or any combination thereof is by fluorescence resonance energy transfer or bioluminescence resonance energy transfer.

### EXAMPLES

### Example 1: Fecal Transplant in Mice from ASD and NT Human Donors and Analysis of Microbial Taxa

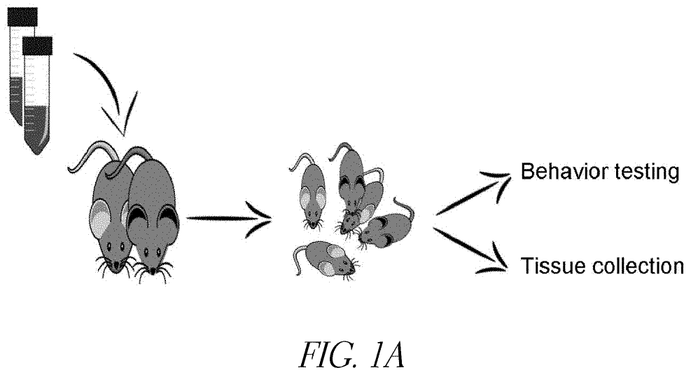

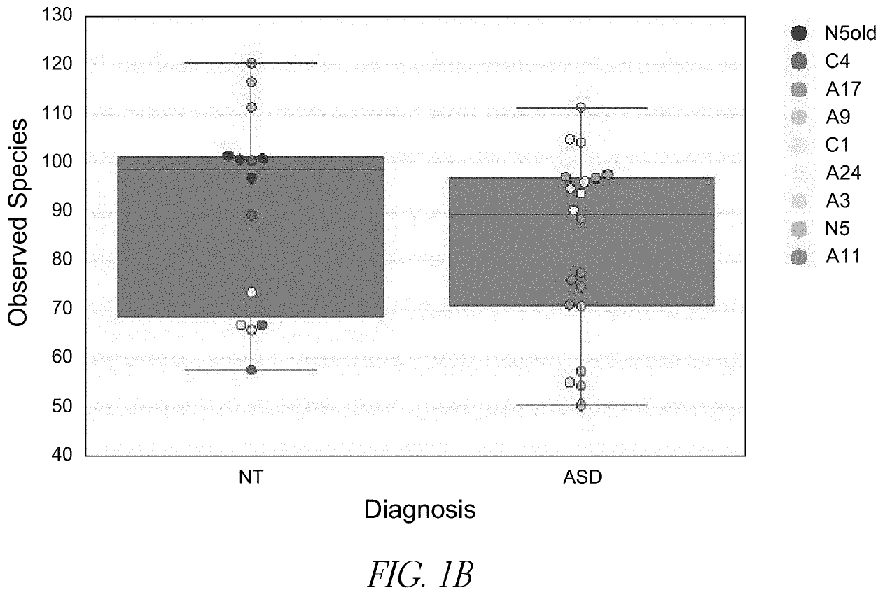

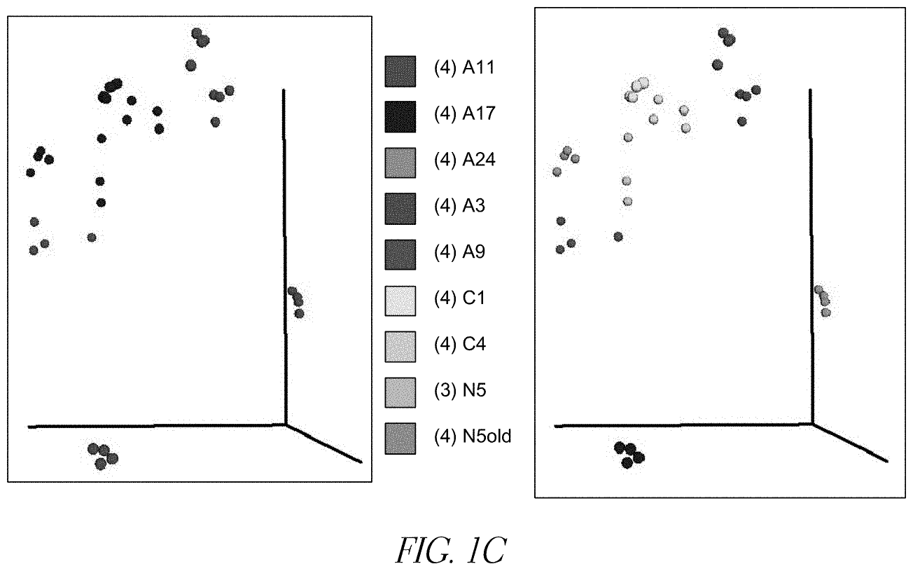

The gastrointestinal tract of wild-type germ-free mice were inoculated with fecal samples from human typically developing (TD)(which may also be referred to as “neurotypicaly” or “NT”) subjects, or human subjects diagnosed with ASD. The origins of the fecal samples used for the transplants are summarized in Table 2. The scheme is illustrated schematically in FIGS. 1A-D. Wild type (C57Bl/6) Mice were colonized at weaning, bred at 7-8 weeks of age and their adult offspring were behaviorally tested or sacrificed for tissue harvest (FIG. 1A). Alpha and Beta Diversity from amplicon-based (Deblur) analysis of the colonic bacterial community (FIGS. 1B-C). General locomotion (by distance traveled in open field testing), repetitive behavior (by marble burying), sociability (by Direct social behavior), and communication (by USV) were tested (FIG. 1D).

TABLE 2
Origins of fecal samples transplanted into experimental germ-free mice

| | | | | GSI | Std ADOS | | |
|---|---|---|---|---|---|---|---|
| Group | Sample | Gender | Age | score | score | ATEC | PDDB |
| NT | N5-old | M | 4 | 0 | N/A | N/A | N/A |
| NT | C11-new | M | 5 | N/A | N/A | N/A | N/A |
| NT | C4-New | M | 6 | 0 | N/A | N/A | N/A |
| NT | C1-new | M | 9 | 0 | N/A | N/A | N/A |
| NT | C5-new | M | 9 | 0 | N/A | N/A | N/A |
| ASD-Mild | A9-new | M | 6 | 2 | 4 | 24 | −156 |
| ASD-Mild | A8-old | M | 6 | 4 | 4 | 52 | −58 |
| ASD-Mild | A8-New | M | 9 | 9 | 4 | 72 | −7 |
| ASD-Severe | A15-old | M | 3 | 7 | 8 | 86 | 27 |
| ASD-Severe | A24-new | M | 4 | 8 | 8 | 127 | 49 |
| ASD-Severe | A9-old | M | 4 | 5 | 6 | 101 | N/A |
| ASD-Severe | A11-old | M | 5 | 5 | 7 | 110 | −17 |
| ASD-Severe | A7-old | M | 6 | 3 | 6 | 58 | −127 |
| ASD-Severe | A3-old | M | 8 | 2 | 6 | 106 | 27 |
| ASD-Severe | A10-new | M | 9 | 9 | 6 | 100 | −54 |
| ASD-Severe | A17-old | M | 11 | 7 | 9 | 98 | N/A |
| NT | | | 6.6 ± 1.03 | 0.5 ± 0.5 | N/A | N/A | N/A |
| ASD-Mild | | | 7 ± 1 | 5 ± 2.08 | 4 ± 0 | 49.3 ± 13.9 | −73.7 ± 43.7 |
| ASD-Severe | | | 6.25 ± 1 | 5.75 ± 0.86 | 7 ± 0.42 | 98.3 ± 7.1 | −15.8 ± 26.9 |

The mice were assessed for ASD-like behaviors, for example, social interactions such as three-chamber social interest and social novelty interest, caged first-contact latency and time in contact; communication such number of ultrasonic vocalizations, repertoire, and acoustic structure of pup isolation calls, male behavior, and same-sex interactions; and repetitive behaviors such a self-grooming, jumping, and marble burying.

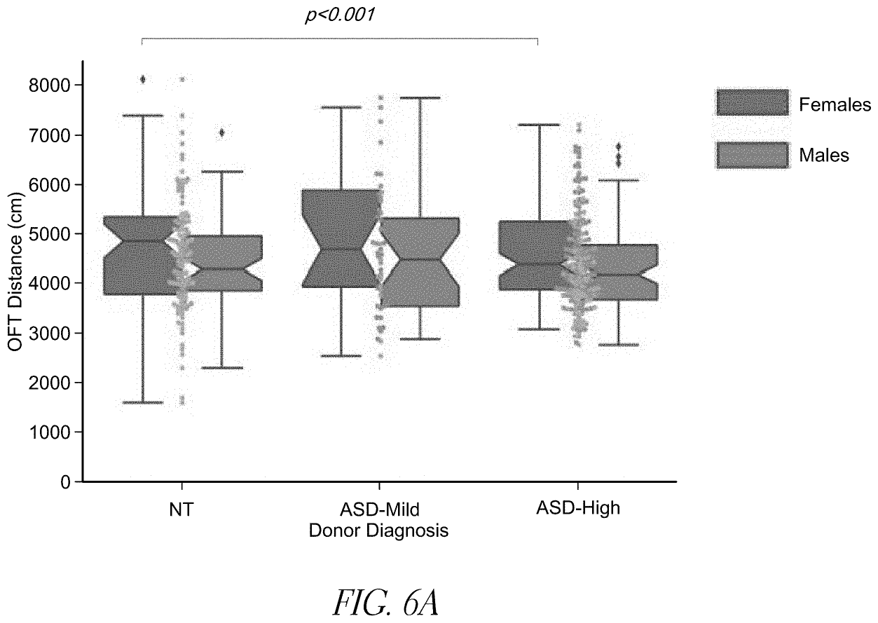

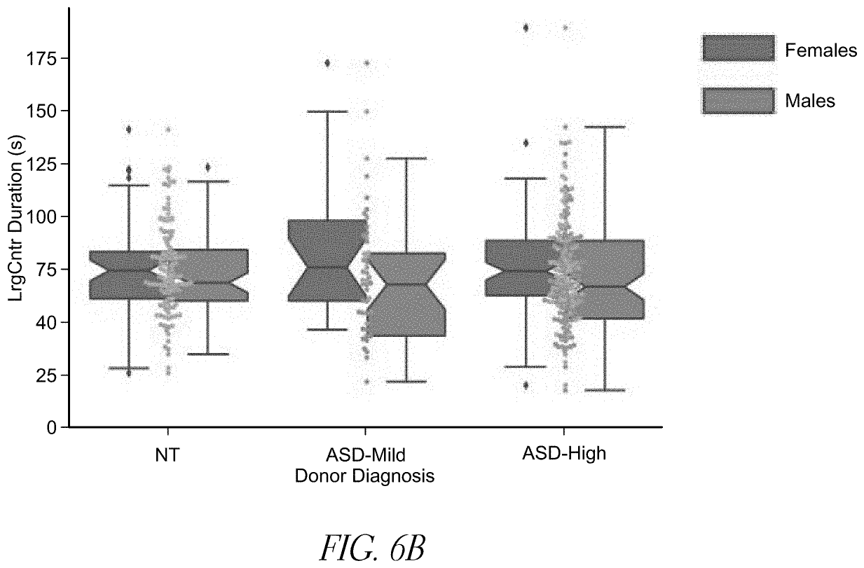

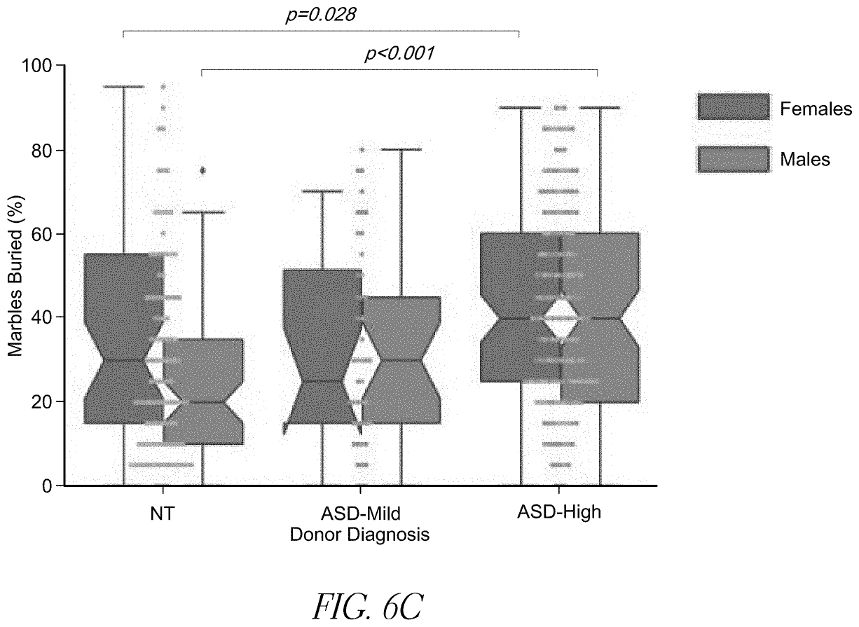

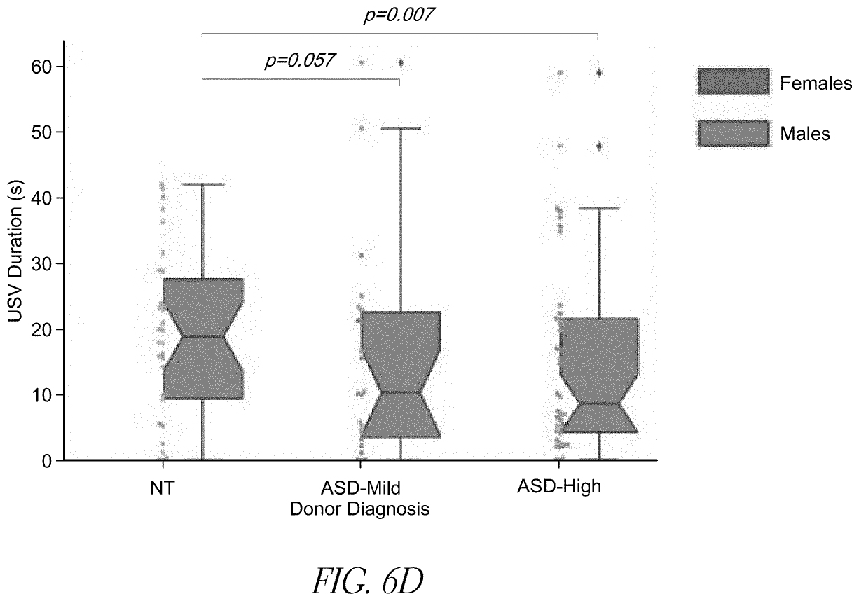

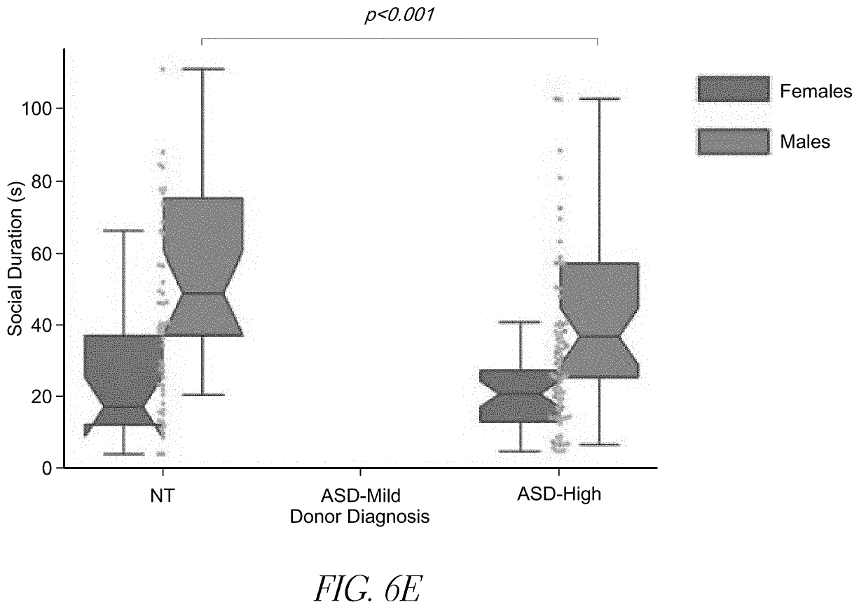

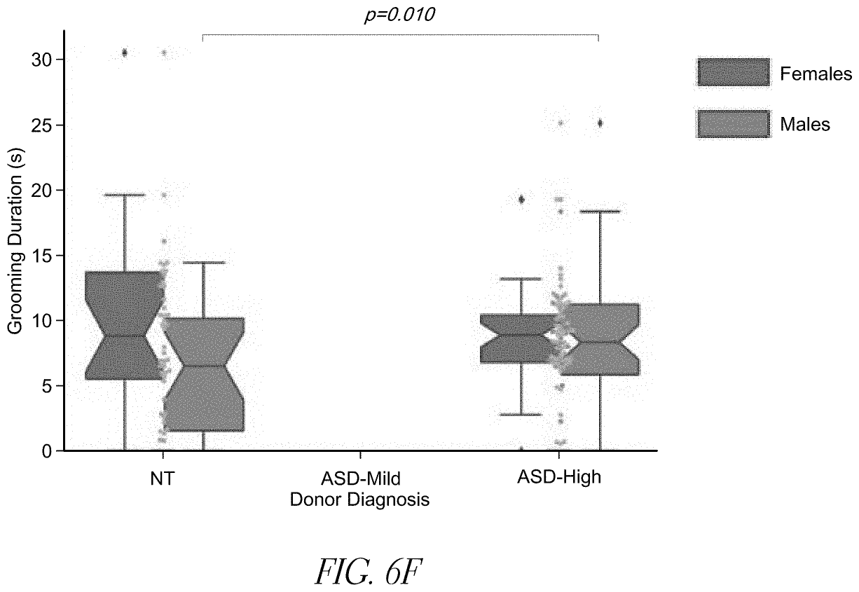

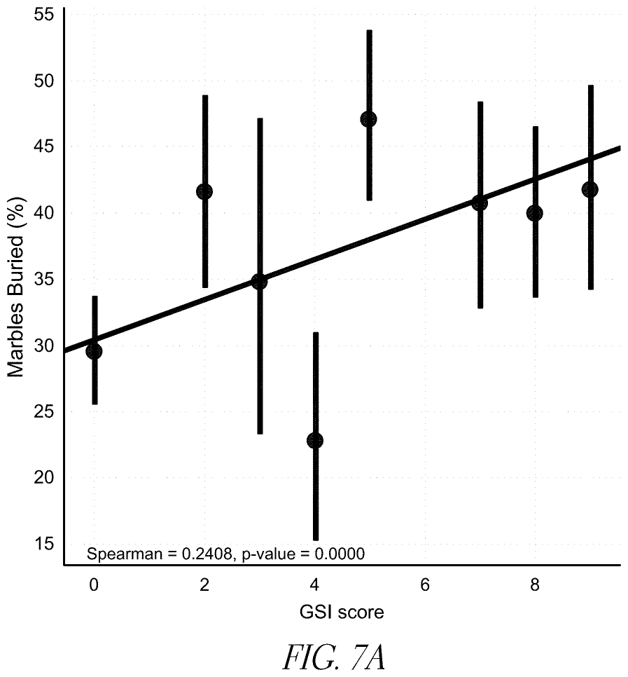

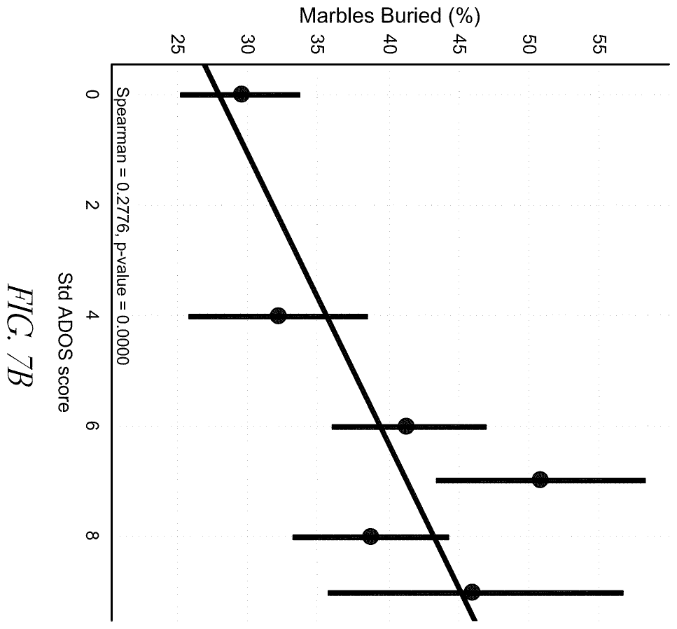

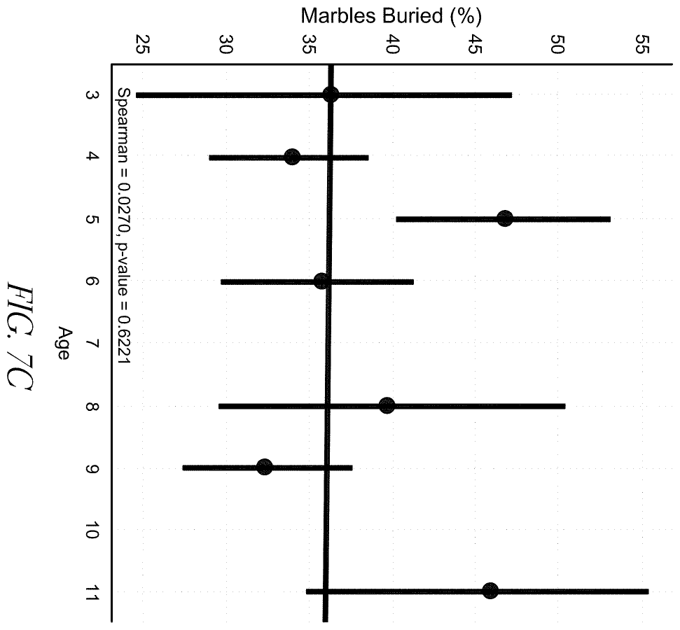

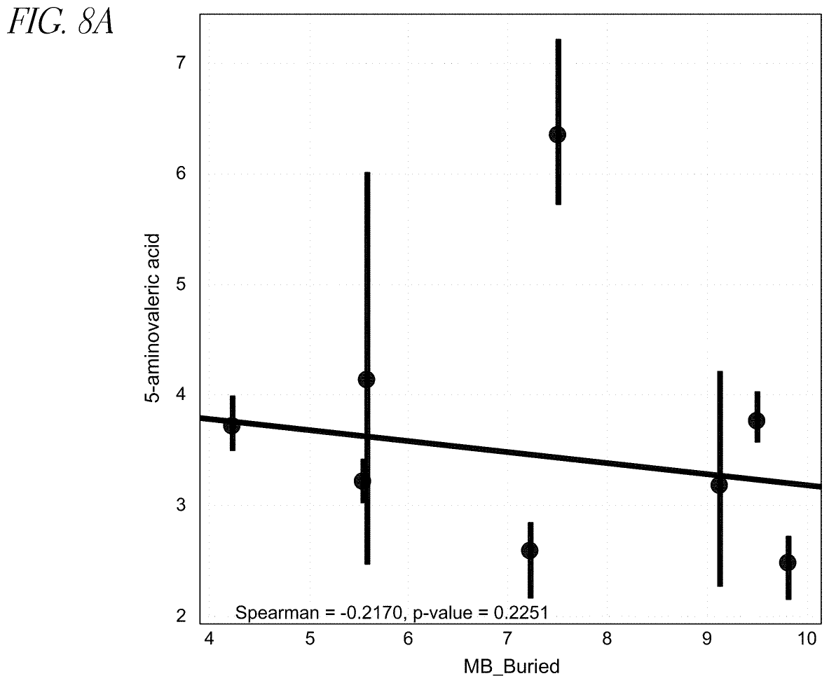

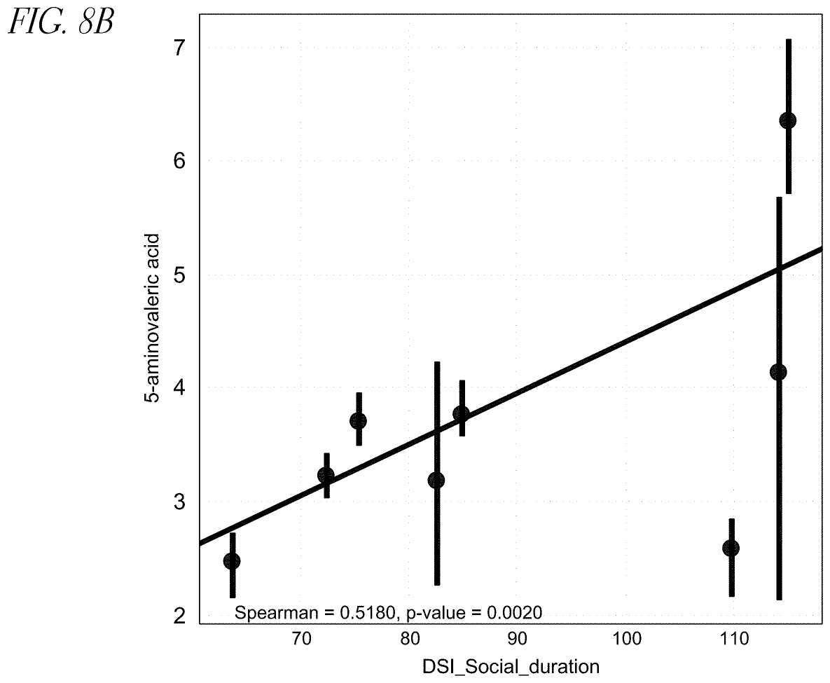

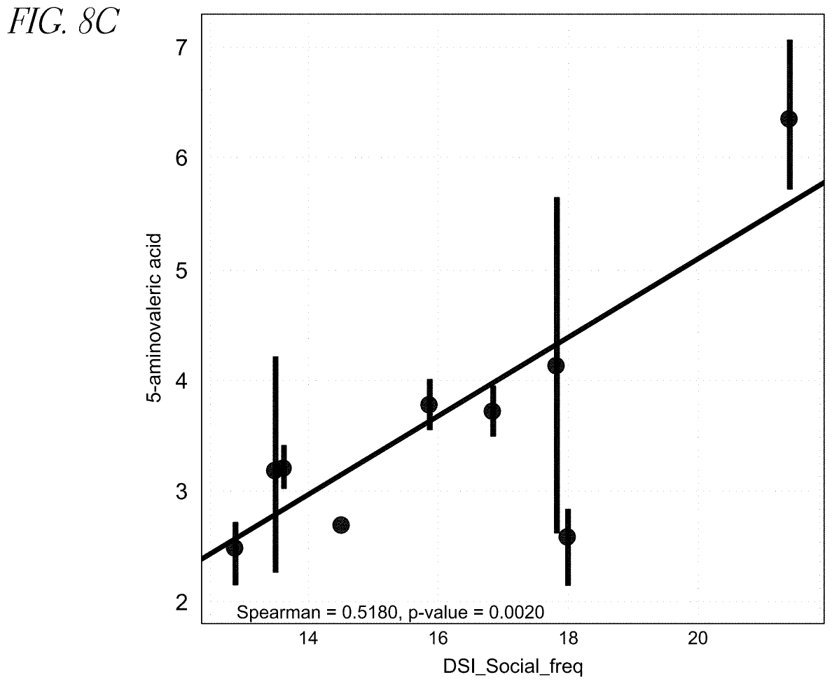

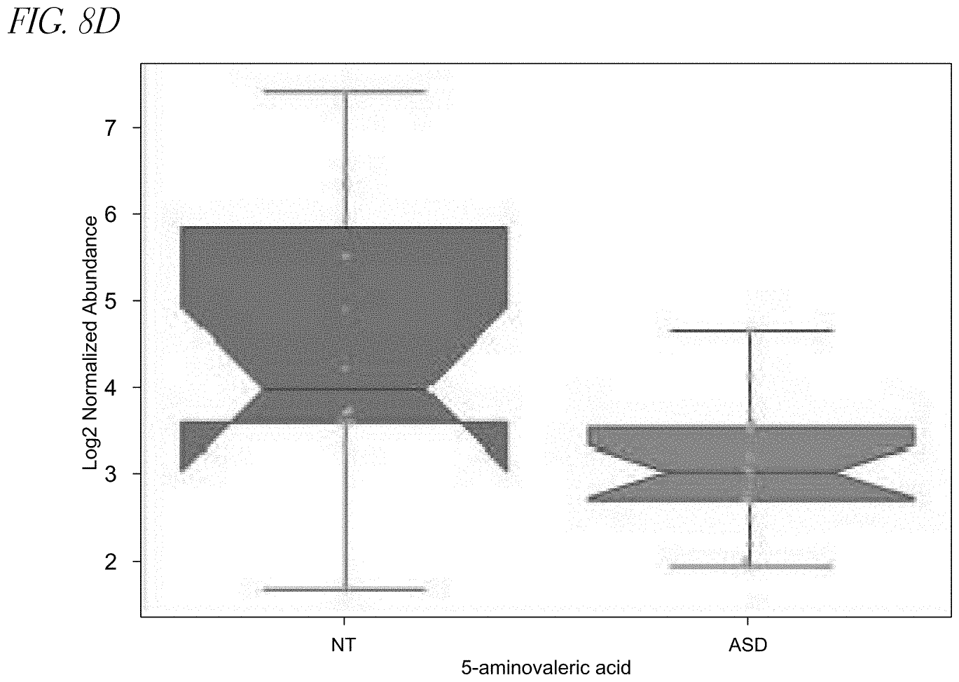

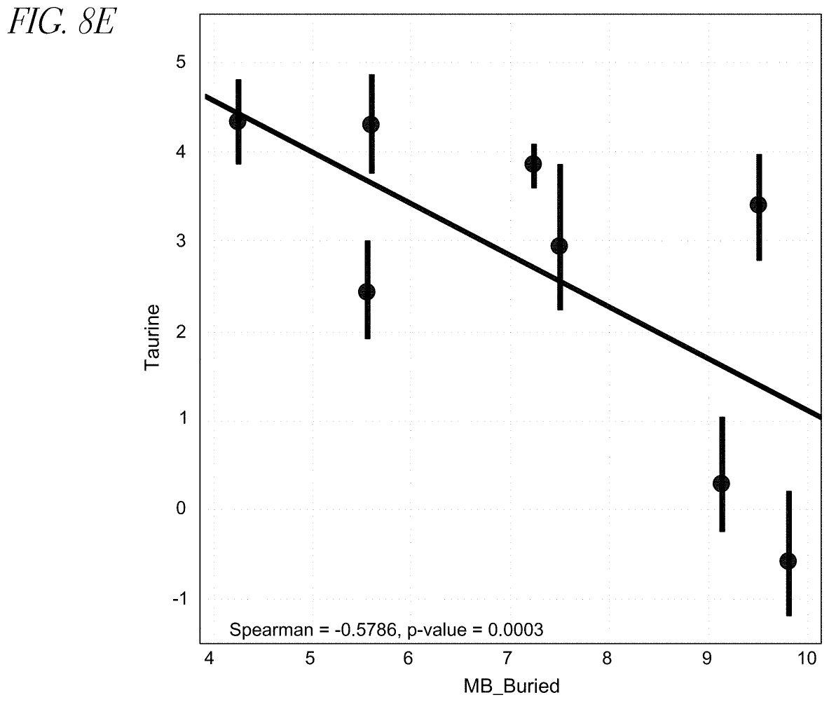

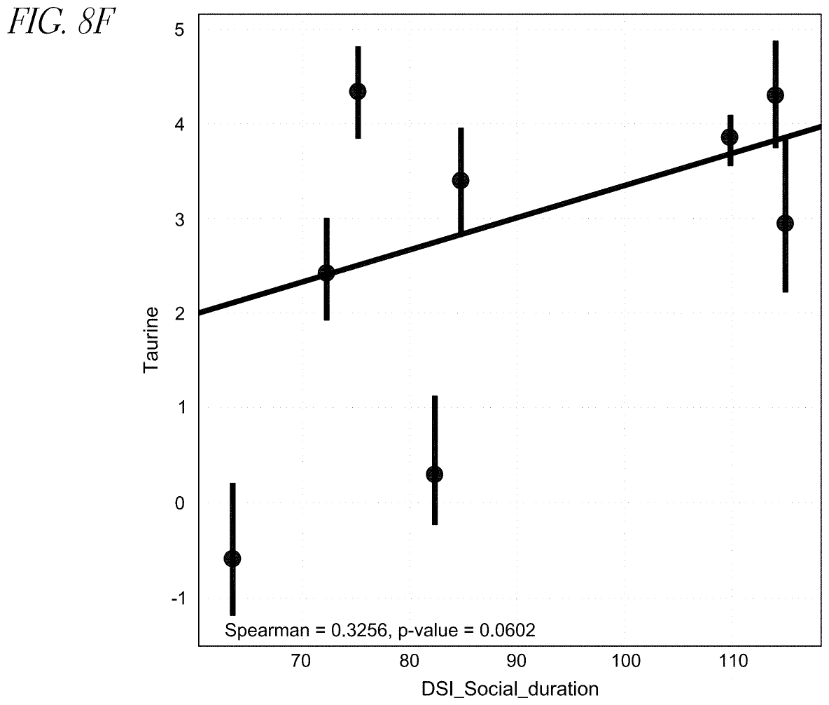

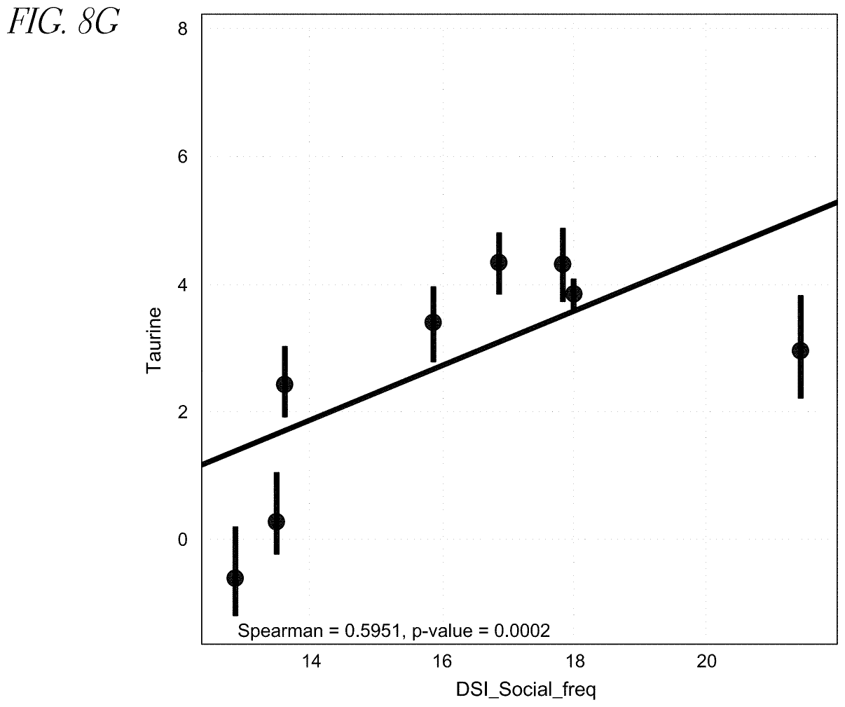

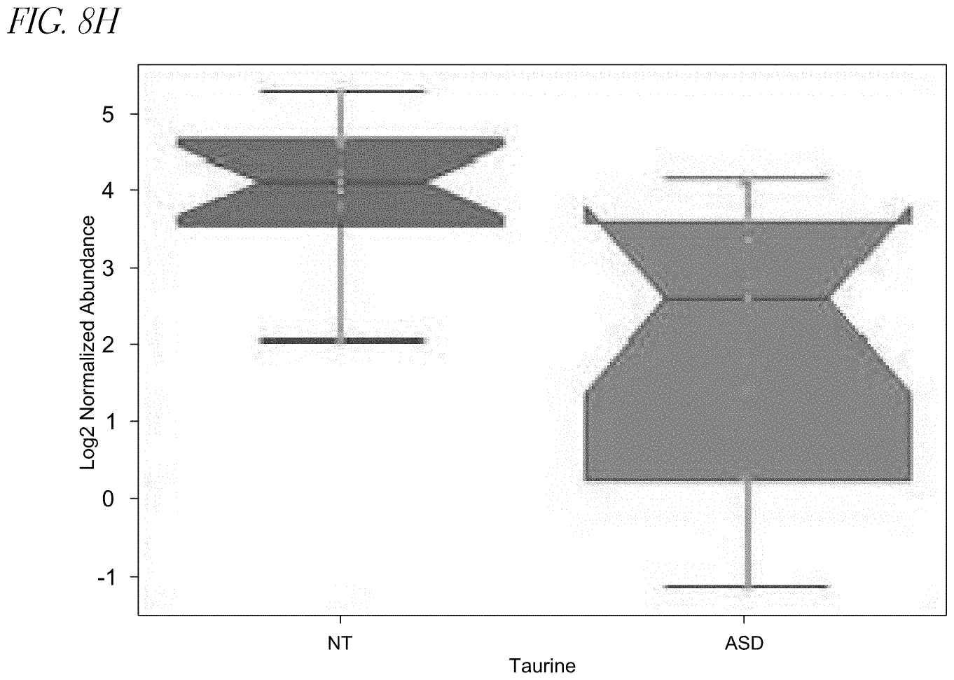

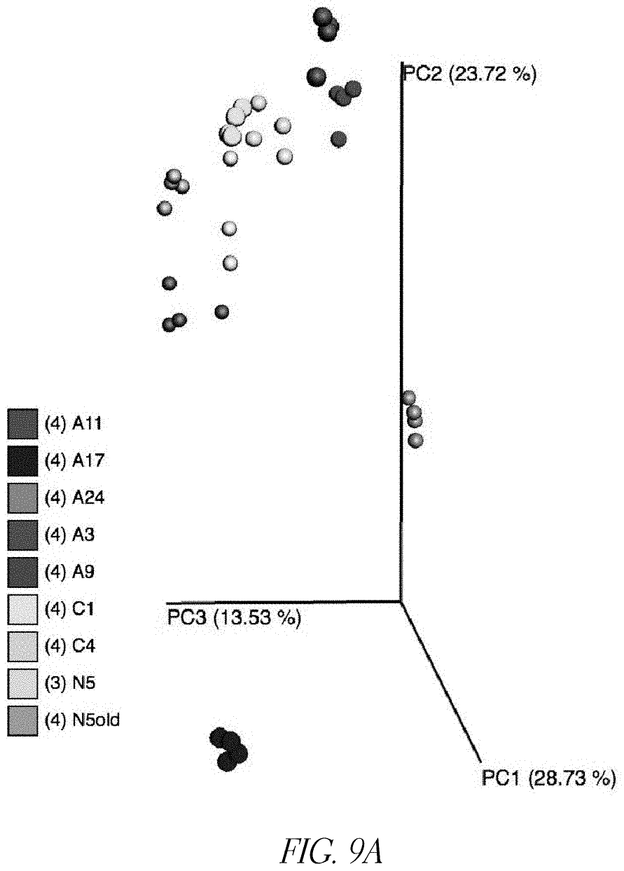

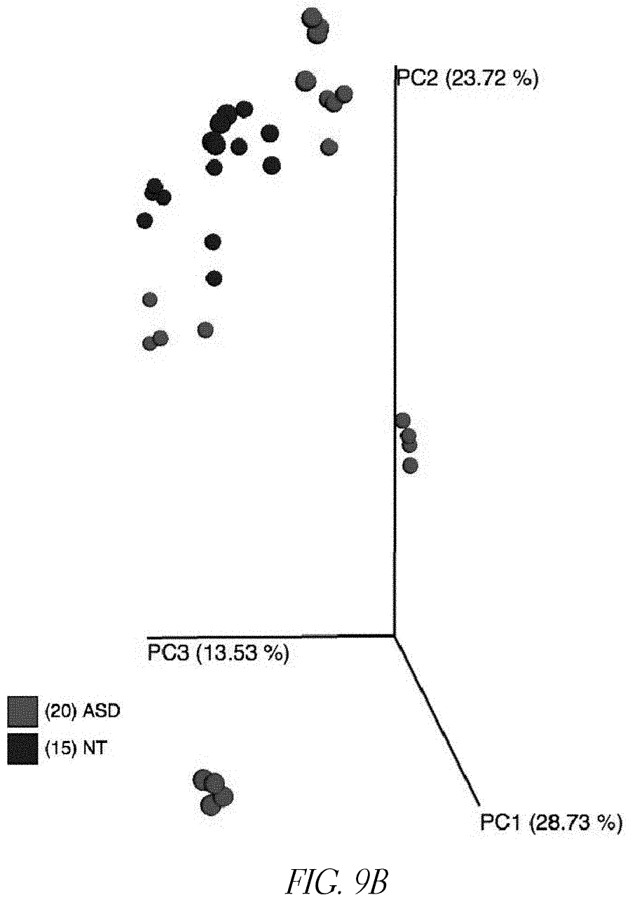

As shown in FIGS. 6A-B, ASD-colonized mice exhibited activity and anxiety, as measured by OFT Distance (FIG. 6A) and Center Durations (FIG. 6B). Furthermore, as shown in FIGS. 6C-F, ASD mice exhibited ASD core behavioral deficits, as measured by marble burying (FIG. 6C), USC Duration(s) (FIG. 6D), Social Durations (FIG. 6E), Grooming Durations and (FIG. 6F). Notably, the repetitive behaviors in the ASD-colonized mice correlated correlate with patient clinical data (See FIGS. 7A-C). Thus, ASD core behaviors were enhanced in the ASD-colonized mice compared to NT-colonized controls, including activity and anxiety (FIGS. 6A-B), and ASD core behavioral deficits (FIGS. 6C-F). Moreover, repetitive behaviors in ASD-colonized mice correlate with patient clinical data (FIGS. 7A-C). Additionally, neuroactive fecal metabolites correlated with behavioral outcomes (FIGS. 8A-H). Further corroborating the fidelity of the fecal transplant, it was observed h humanized mice cluster by donor (FIGS. 9A-B). Thus, fecal transplant in accordance with some embodiments herein induces ASD core behaviors in mice.

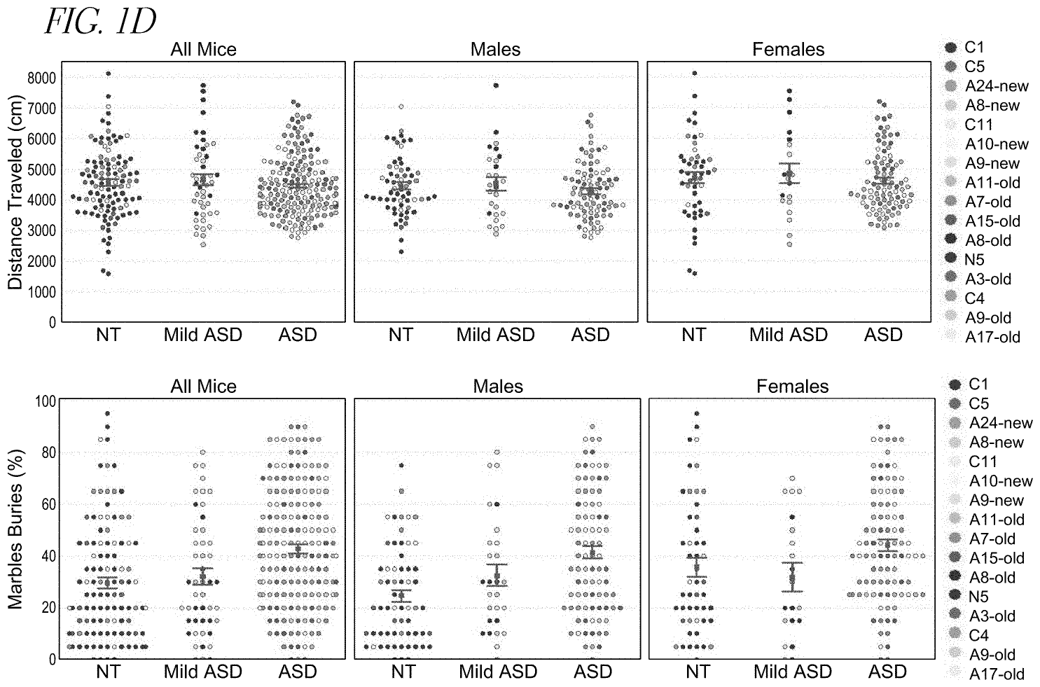

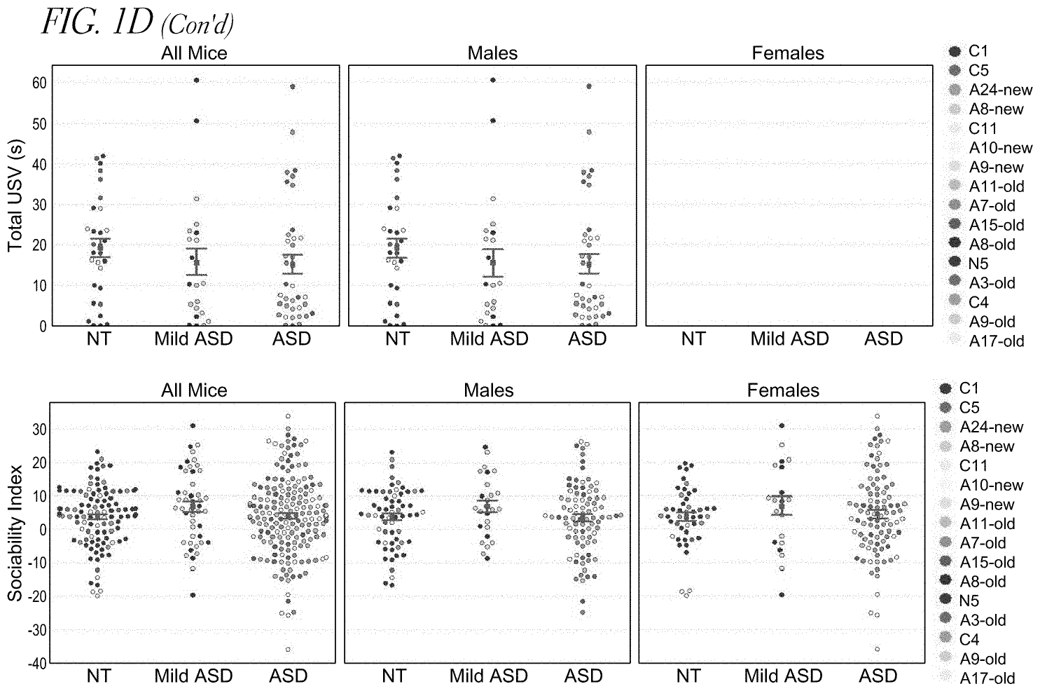

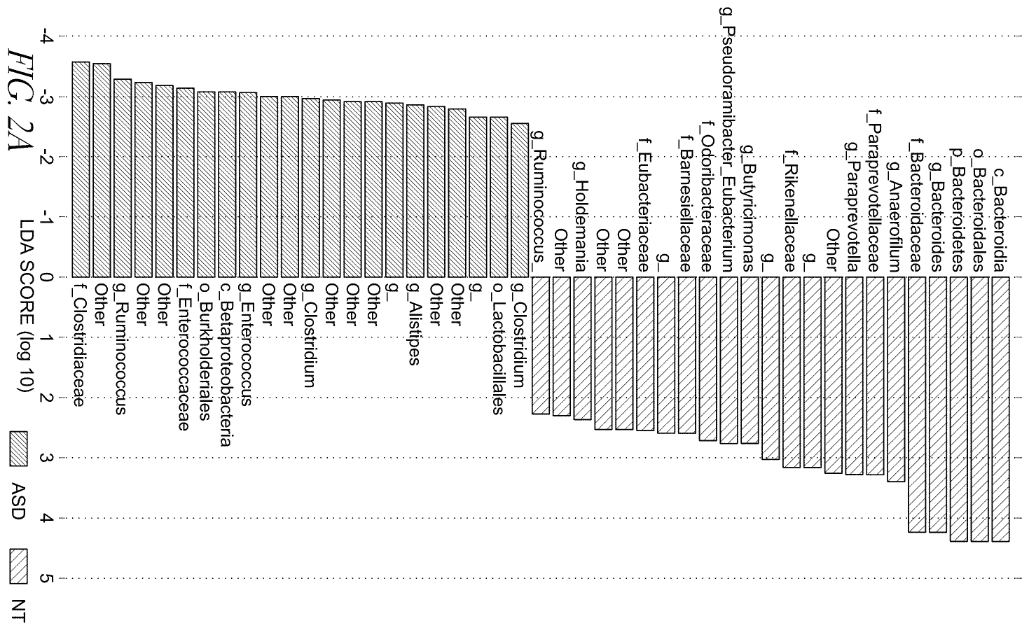

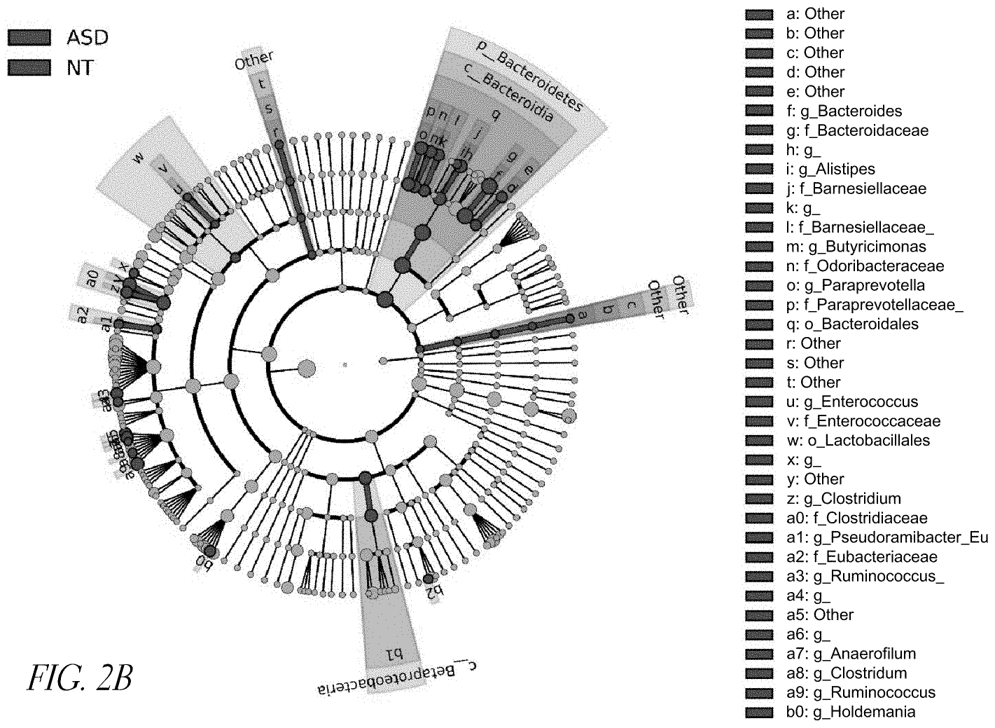

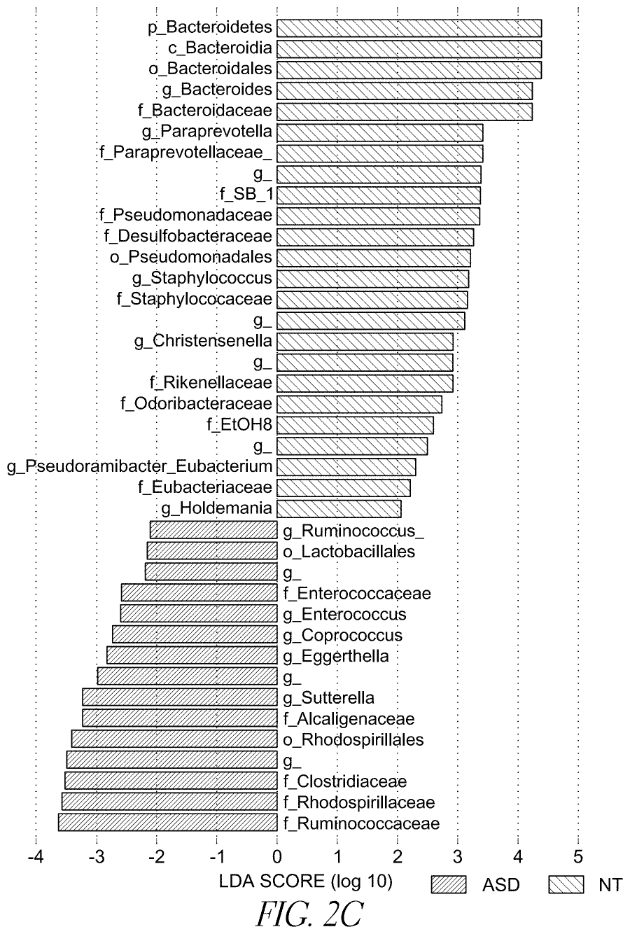

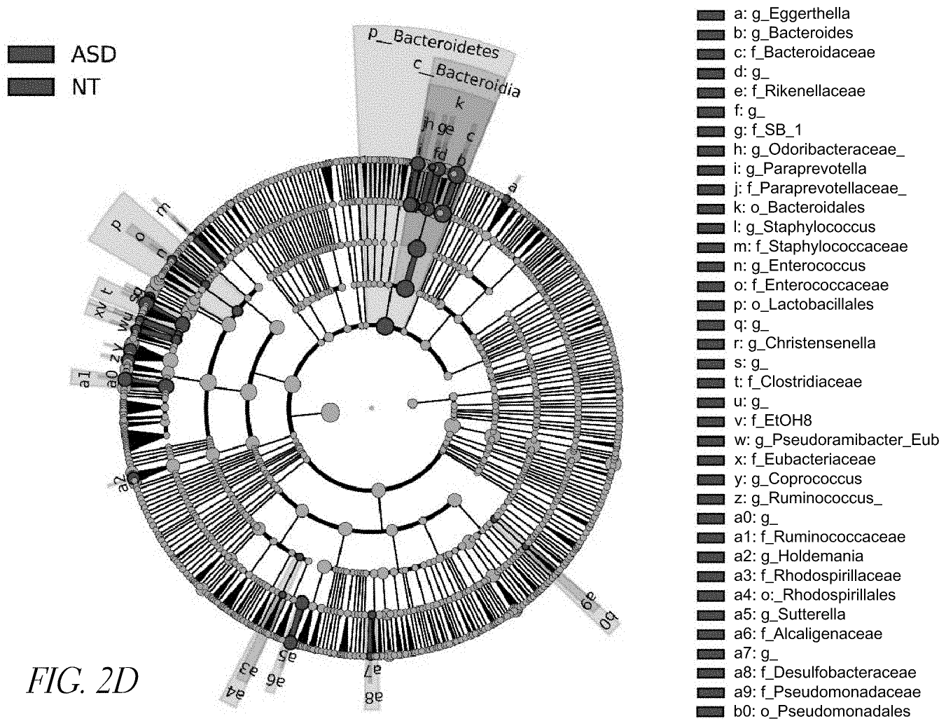

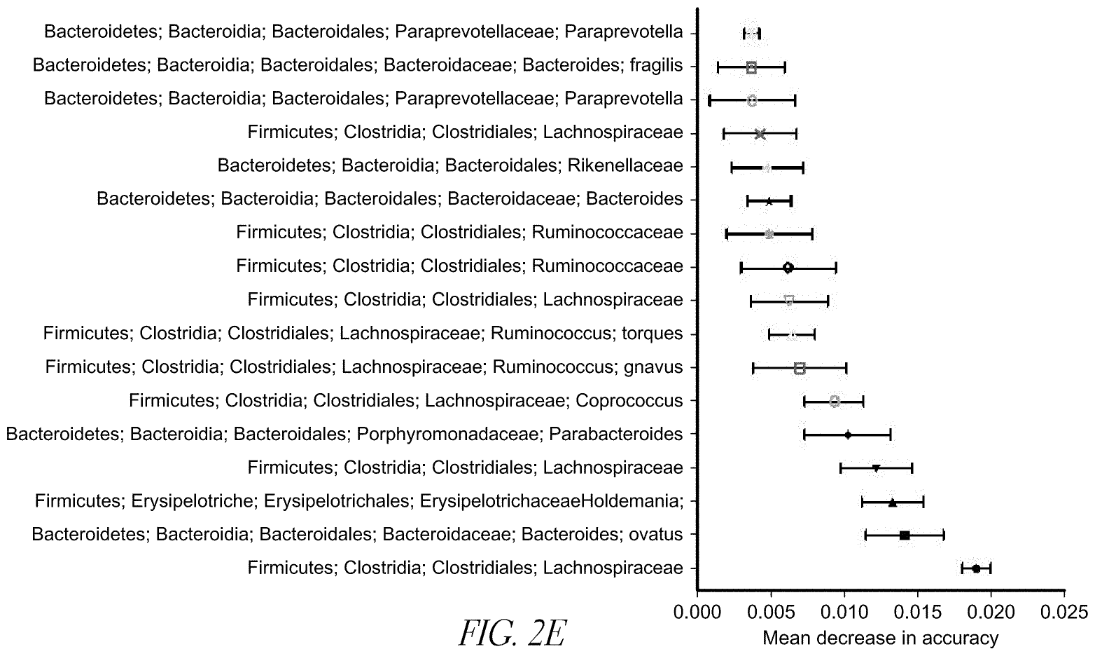

Bacterial 16S sequences were obtained from fecal samples of the NT- and ASD-colonized mice, and discriminatory bacteria between NT- and ASD-colonized mice were identified by a linear discriminant analysis (LefSe; FIGS. 2A-B) and by a machine learning algorithm (Random Forest; FIG. 2C). As shown in FIGS. 2A-C (and summarized in Tables 1A and 1B), bacterial composition differed between the gastrointestinal tracts of ASD- and NT-colonized mice. The 16S sequences that differed between ASD-colonized and NT-colonized control mice are shown in FIGS. 10A-T (SEQ ID NOs: 1-20). Table 3 summarized the 16S sequences (and corresponding taxa) that discriminated between the gastrointestinal tracts of ASD-colonized and NT-colonized mice.

TABLE 3

| | SEQ | Mean | |
|---|---|---|---|
| | ID | decrease in | Standard |
| Taxonomy | NO: | accuracy | deviation |
| k_Bacteria; p_Firmicutes; c_Clostridia; | 1 | 0.0235293 | 0.00343509 |
| o_Clostridiales; f_Lachnospiraceae | | | |
| k_Bacteria; p_Bacteroidetes; | 2 | 0.0143774 | 0.00310101 |
| c_Bacteroidia; o_Bacteroidales; | | | |
| f_Bacteroidaceae; g_Bacteroides; s_ovatus | | | |
| k_Bacteria; p_Firmicutes; c_Clostridia; | 3 | 0.01357476 | 0.00278779 |
| o_Clostridiales; f_Ruminococcaceae | | | |
| k_Bacteria; p_Firmicutes; c_Clostridia; | 4 | 0.0130122 | 0.0022974 |
| o_Clostridiales; f_Ruminococcaceae | | | |
| k_Bacteria; p_Firmicutes; | 5 | 0.01264792 | 0.00439758 |
| c_Erysipelotrichi; o_Erysipelotrichales; | | | |
| f_Erysipelotrichaceae; g_Holdemania; s_{—} | | | |
| k_Bacteria; p_Firmicutes; c_Clostridia; | 6 | 0.01074817 | 0.00167224 |
| o_Clostridiales; f_Lachnospiraceae | | | |
| k_Bacteria; p_Bacteroidetes; | 7 | 0.01015321 | 0.00200987 |
| c_Bacteroidia; o_Bacteroidales; | | | |
| f_Porphyromonadaceae; g_Parabacteroides; | | | |
| s_{—} | | | |
| k_Bacteria; p_Firmicutes; c_Clostridia; | 8 | 0.00841908 | 0.00159917 |
| o_Clostridiales; f_Lachnospiraceae | | | |
| k_Bacteria; p_Firmicutes; c_Clostridia; | 9 | 0.00802073 | 0.00209179 |
| o_Clostridiales; f_Lachnospiraceae; g_; | | | |
| s_{—} | | | |
| k_Bacteria; p_Bacteroidetes; | 10 | 0.00682282 | 0.00187533 |
| c_Bacteroidia; o_Bacteroidales; | | | |
| f_Bacteroidaceae; g_Bacteroides; s_{—} | | | |
| k_Bacteria; p_Bacteroidetes; | 11 | 0.00619951 | 0.00153712 |
| c_Bacteroidia; o_Bacteroidales; | | | |
| f_Rikenellaceae; g_; s_{—} | | | |
| k_Bacteria; p_Firmicutes; c_Clostridia; | 12 | 0.0059207 | 0.00128334 |
| o_Clostridiales; f_Ruminococcaceae; g_; | | | |
| s_{—} | | | |
| k_Bacteria p_Bacteroidetes; c_Bacteroidia; | 13 | 0.00585671 | 0.00180951 |
| o_Bacteroidales; f_[Paraprevotellaceae]; | | | |
| g_Paraprevotella; s_{—} | | | |
| k_Bacteria; p_Firmicutes; c_Clostridia; | 14 | 0.00547138 | 0.00131209 |
| o_Clostridiales; f_Lachnospiraceae; | | | |
| g_Coprococcus; s_{—} | | | |
| k_Bacteria; p_Bacteroidetes; | 15 | 0.00514705 | 0.00144205 |
| c_Bacteroidia; o_Bacteroidales; | | | |
| f_[Paraprevotellaceae]; g_Paraprevotella; | | | |
| s_{—} | | | |
| k_Bacteria; p_Bacteroidetes; | 16 | 0.00510033 | 0.00088943 |
| c_Bacteroidia; o_Bacteroidales; | | | |
| f_Rikenellaceae; g_; s_{—} | | | |

It was further observed that the metabolome of ASD-colonized mice differs from that of NT-colonized mice. Feces and serum samples from colonized mice were analyzed by GC-MS or NMR. Significantly different metabolites between groups were identified, as presented by volcano plots (FIG. 3A). The normalized abundance of these metabolites, by mouse and donor (FIG. 3B). Pathway analysis (FIG. 3C), performed by MetaboAvalyst 3.0 shows significant differences in amino-acid metabolism and biosynthesis (in GCMS, top panel and NMR, bottom panel). Additionally, neuroactive fecal metabolites correlate with behavioral outcomes (FIGS. 8A-H).

### Example 2: Fecal Transplant in Mice from ASD and NT Human Donors and Analysis of Microbial Taxa

A further analysis of microbial taxa in ASD fecal transplant mice was performed.

Frozen mouse fecal samples were shipped overnight on dry ice and stored in −80° C. until DNA extraction. Human feces that were used as donor samples for the mouse experiments were also shipped back to ASU in order to be processed for microbial DNA extraction and next-generation sequencing together with mouse fecal samples. At ASU, microbial genomic DNA was extracted from fecal samples using the PowerSoil® DNA Isolation Kit (Mobio Carlsbard, Calif.) with a modification based on the manufacturer protocol. Quality and quantity of genomic DNA was verified using a NanoDrop ND-1000 spectrophotometer (NanoDrop Technology, Rockland, Del.). Qualified genomic DNA samples were processed for 16S rRNA library preparation and next-generation sequencing at the Microbiome Analysis Laboratory in the Biodesign Swette Center for Environmental Biotechnology (accessible on the world-wide-web). The Earth Microbiome Project standard protocols (accessible on the world-wide-web) were employed with the barcoded primer set 515F-806R (515F: GTGCCAGCMGCCGCGGTAA (SEQ ID NO: 1), 2086R: GGACTACHVGGGTWTCTAAT (SEQ ID NO: 2)) that targets the V4 region of the bacterial (and archeal) 16S rRNA gene (Caporaso et al., 2012). Paired-end, 2×150 bp, next-generation sequencing was performed using MiSeq Illumina platform (MiSeq Reagent Kit v2; Illumina Inc., San Diego, Calif.) and microbiome sequencing data were analyzed using the Quantitative Insights Into Microbial Ecology (QIIME) software package.

Demultiplexed sequencing outputs were obtained from the ASU sequencing facility and analyzed using the QIIME 2 (version 2017.9) software package according to the suggested workflow (Caporaso et al., 2010). Since there was little overlap between forward and reverse reads, only forward reads (˜150 bp long) were used for subsequent analysis. Primers were first trimmed from the reads and sub-operational-taxonomic units (sOTUs) were obtained using the Deblur denoising plugin (Amir et al., 2017) on reads trimmed to 120 bp. Subsequently, alignments were obtained using MAFFT (Yamada et al., 2016) and a phylogenetic tree was generated using FastTree (Price et al., 2009). Alpha and Beta diversities were analyzed using the core-metrics-phylogenetic for observed OTUs, Faith's phylogenetic diversity, and Pielou's evenness measures for alpha diversity and unweighted Unifrac and Bray-Curtis for beta diversity measures (Lozupone and Knight, 2005). Taxonomic analysis was performed using the q2-feature-classifier trained on GreenGenes 13_8 99% OTU table (McDonald et al., 2012). Differential abundance analysis was performed using the Phyloseq (1.20.0) and DESeq2 (1.16.1) R packages (Love et al., 2014; McMurdie and Holmes, 2013). To further analyze sOTUs that contribute to the discrimination between NT and ASD samples and to behavioral phenotypes, a RandomForest analysis (Liaw et al., 2002), as implemented in QIIME 2, was used. Per sample metagenomics prediction was done using PICRUSt. For compatibility with the PICRUSt package (Langille et al., 2013), closed-reference OTU tables were generated by clustering sOTUs with the GreenGenes 13_5 99% OTU map in QIIME. Predicted metagenomes were then generated according to the suggested workflow for PICRUSt 1.1.2.

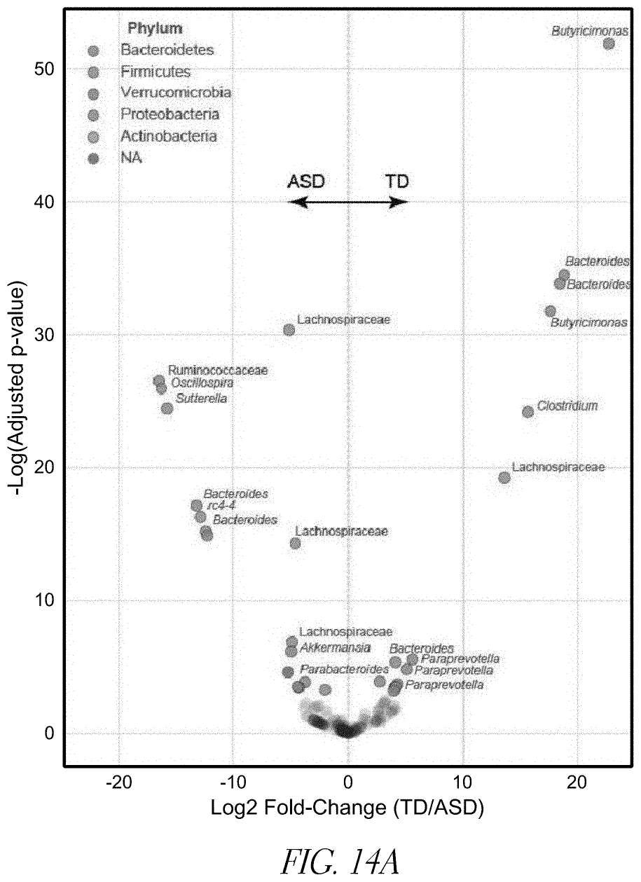

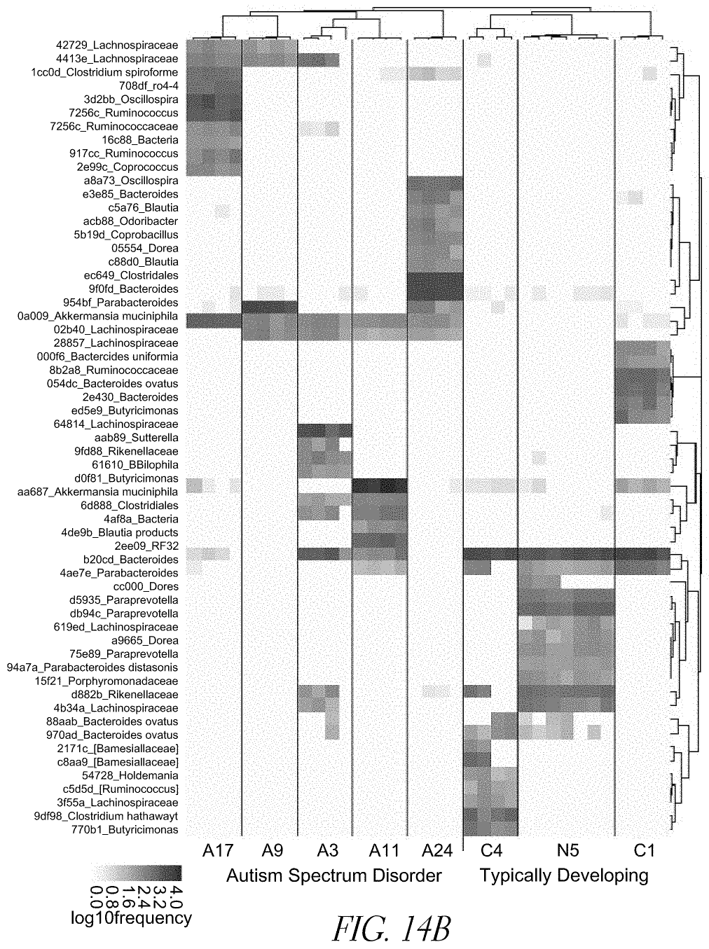

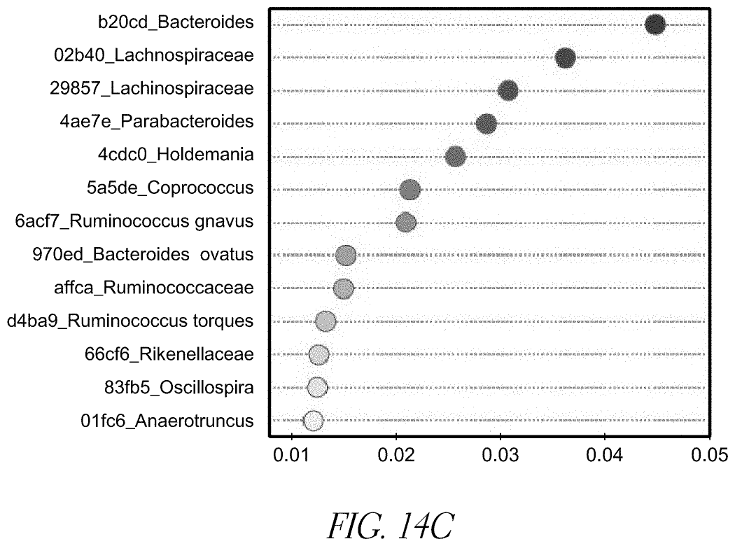

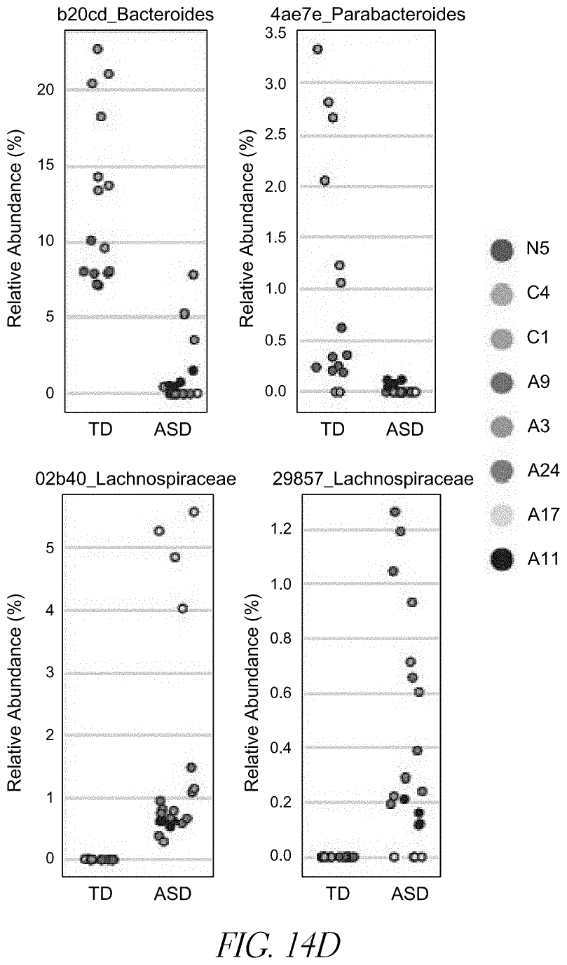

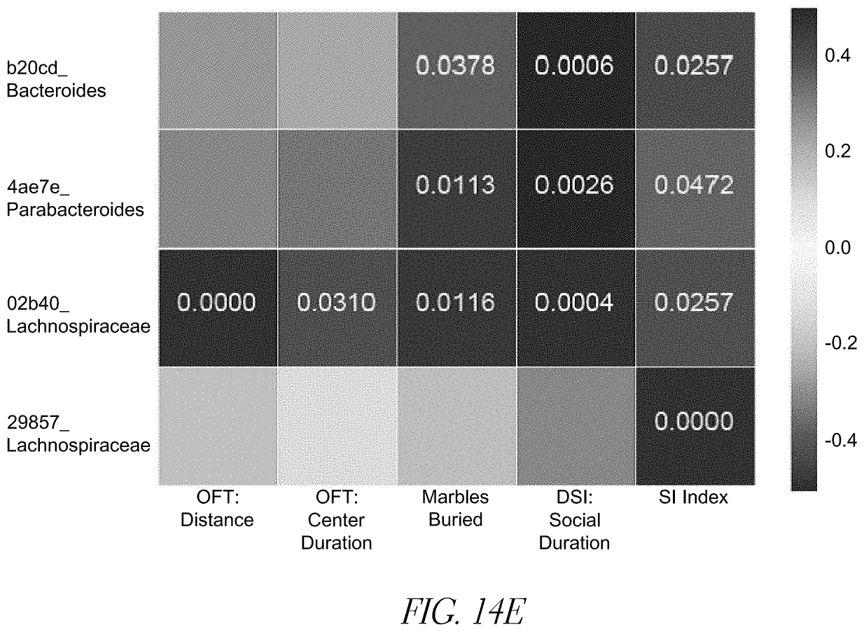

In total, 31 sub-operational taxonomic units (sOTUs) are differentially abundant between groups (FIGS. 14A-14B). Those sOTUs belong predominantly of the Clostridia and Bacteroidia classes, as well as Verrucomicrobia, alpha and beta Proteobacteria with a single representative of each. sOTUs for Bacteroidia are associated with most controls. Specifically, Bacteroides ovatus, Parabacteroides merdae, and an sOTU closely related to Bacteroides thetaiotaomicron, are prevalent in all TD samples, and absent from ASD samples. Conversely, the Lachnospiraceum Eisenbergiela tayi is prevalent among all ASD recipients, and absent from TD groups (FIG. 2B). These observations were further corroborated by an unsupervised classification analysis using RandomForest. The trained classifier assigned all offspring samples correctly by group (TD/ASD; accuracy ratio over baseline: 1.75). The trained model indicates 13 sOTUs contributing >1% to discrimination between TD and ASD samples (FIG. 14C), including E. tayi, B. ovatus, and P. merdae (FIG. 14D). This differential abundance analysis indicates that several species of Bacteroides and Parabacteroides are exclusively present in TD samples, while different sOTUs that are present only in ASD belong to E. tayi (Lachnospiraceae) (FIG. 14D).

Spearman correlations were performed to test whether discrete sOTUs positively or negatively co-vary with behavioral outcomes: The abundance of four bacterial sOTUs significantly correlates with both repetitive and social behaviors in mice (FIG. 2E). The Bacteroides species (b20cd_Bacteroides; maps to several different Bacteroides species) and P. merdae (4ae7e_Parabacteroides) both correlate with reduced repetitive behavior (less marbles buried) and increased social behavior (longer time socializing in both DSI and 3-chamber sociability). Conversely, the E. tayi sOTUs (02b40_Lacnospiraceae and 29857_Lacnospiraceae) show the opposite effects, as they correlate with increased repetitive behavior and social interaction deficits (FIG. 14E). The association of specific bacterial species (sOTUs) with TD or ASD samples that are also highly correlated with ASD-relevant behaviors supports the hypothesis that specific bacteria contribute to the etiology of ASD.

Accordingly, it is shown that the presence of Bacteroides species and P. merdae correlate with reduced repetitive behavior and increased social behavior. On the other hand, E. tayi sOTUs correlates with symptoms of ASD, including increased repetitive behavior and social interaction deficits. Accordingly, in some embodiments, ASD, or a risk or severity thereof can be identified based on a presence of E. tayi and/or an absence of Bacteroides species and/or P. merdae in a sample of a subject's gut (e.g. a fecal sample).

In at least some of the above-described embodiments, one or more elements used in an embodiment can interchangeably be used in another embodiment unless such a replacement is not technically feasible. It will be appreciated by those skilled in the art that various other omissions, additions and modifications may be made to the methods, compositions, kits, and uses described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a device or substrate having at least one of A, B, and C” would include but not be limited to devices or substrates that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a device or substrate having at least one of A, B, or C” would include but not be limited to devices or substrates that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Additionally, when a method comprising a composition or product (for example a binding agent) is described herein, the corresponding or composition for use is also contemplated. For example, a method comprising contacting with a biological sample to detect a presence or absence of a bacteria listed in Table 1A and/or Table 1B, expressly also contemplated a binding agent for use in detecting a presence or absence of a bacteria listed in Table 1A and/or Table 1B

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

1-92. (canceled)

93. A method for identifying a subject that is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof, comprising detecting one or more binding targets from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, ruminococcaceae, parabacteroides, or eisenbergiela, or any combination thereof in a biological sample from said subject, wherein the absence of, or lower level than a neurotypical control of one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, parabacteroides or wherein the presence of, or higher level than a neurotypical control of lactobacillales, enterococcaceae, Enterococcus, Coprococcus, eggerthela, sutterela, alcaligenaceae, rhodospirillales, clostridaceae, rhodospirillaceae, ruminococcaceae, or eisenbergiela, or any combination thereof, in said biological sample indicates increased risk of developing the condition.

94. The method of claim 93, wherein the binding targets comprise nucleic acids.

95. The method of claim 94, wherein the nucleic acids comprise 16S sequences.

96. The method of claim 93, wherein said detecting one or more binding targets comprises detecting a nucleic acid selected from the group consisting of SEQ ID NO: 1-20.

97. The method of claim 93, wherein nucleic acids of SEQ ID NOs: 1-20 are detected.

98. The method of claim 93, wherein the binding targets comprise antigens.

99. The method of claim 93, wherein the bacteria are selected from the group consisting of the bacteria of Table 1A.1, or two or more bacteria listed in Table 1A.1.

100. The method of claim 93, wherein the bacteria are selected from the group consisting of at least one of the bacteria of Table 1A.2, or two or more bacteria listed in Table 1A.2.

101. The method of claim 93, wherein the bacteria are selected from the group consisting of at least one of the bacteria of Table 1B.1, or two or more bacteria listed in Table 1B.1.

102. The method of claim 93, wherein the bacteria are selected from the group consisting of at least one of the bacteria of Table 1B.2, or two or more bacteria listed in Table 1B.2.

103. The method of claim 93, wherein the bacteria are selected from the group consisting of at least one of the bacteria of Tables 1A.1 and 1B.1, or two or more bacteria listed in any of Tables 1A.1 and 1B.1.

104. The method of claim 93, wherein the bacteria are selected from the group consisting of at least one of the bacteria of Table 1A.1-1A.2 and Tables 1B.1-1B.2, or two or more bacteria listed in any of Tables 1A.1-1A.2 and 1B.1-B.2.

105. A method for identifying a subject that is at risk of having a condition comprising one or more of autism spectrum disorder, anxiety, Parkinson's Disease, Rett Syndrome, Fragile X Syndrome, Tuberous Sclerosis, Multiple Sclerosis, Alzheimer's Disease, Cornelia de Lange Syndrome, stroke, psychiatric diseases, amyotrophic lateral sclerosis, Leukodystrophies including Alexander Syndrome, alpha-synucleinopathies including Lewy Body Dementia, incidental Lewy body disease, Lewy body variant of Alzheimer's disease, multiple system atrophy, pure autonomic failure, or any combination thereof, comprising:
applying a biological sample to a diagnostic device; and
identifying said subject as being at risk of having the condition when an amount of one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, or ruminococcaceae, or any combination thereof captured on the diagnostic device is great than a control amount obtained from a healthy subject.

106. The method of claim 105, wherein detection of said one or more antigens from one or more antigens from one or more of bacteria of the genus Bacteriodetes, bacteroidia, bacteroidales, bacteroidaceae, Paraprevotella, Paraprevotellaceae, pseudomonadaceae, desulfobacteraceae, pseudomonadales, Staphylococcus, staphylococcaceae, Christensenella, rikenellaceae, odonbacteraceae, Pseudoramibacter, eubacteraciaea, Holdemania, Ruminococcus, lactobacillales, enterococcaceae, Enterococcus, Coprococcus, eggerthela, sutterela, alcaligenaceae, rhodospirillales, clostridaceae, rhodospirillaceae, or ruminococcaceae, or any combination thereof is by fluorescence resonance energy transfer or bioluminescence resonance energy transfer.

107. The method of claim 105, wherein the bacteria are selected from the group consisting of the bacteria of Table 1A.1, or two or more bacteria listed in Table 1A.1.

108. The method of claim 105, wherein the bacteria are selected from the group consisting of at least one of the bacteria of Table 1A.2, or two or more bacteria listed in Table 1A.2.

109. The method of claim 105, wherein the bacteria are selected from the group consisting of at least one of the bacteria of Table 1B.1, or two or more bacteria listed in Table 1B.1.

110. The method of claim 105, wherein the bacteria are selected from the group consisting of at least one of the bacteria of Table 1B.2, or two or more bacteria listed in Table 1B.2.

111. The method of claim 105, wherein the bacteria are selected from the group consisting of at least one of the bacteria of Tables 1A.1 and 1B.1, or two or more bacteria listed in any of Tables 1A.1 and 1B.1.

112. The method of claim 105, wherein the bacteria are selected from the group consisting of at least one of the bacteria of Table 1A.1-1A.2 and Tables 1B.1-1B.2, or two or more bacteria listed in any of Tables 1A.1-1A.2 and 1B.1-B.2.


SEQUENCE LISTING (20 sequences)

SEQ ID NO: 1 - DNA, 150 bp, Unknown
  Bacteria of family Lachnospiraceae
tacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctag

SEQ ID NO: 2 - DNA, 150 bp, Bacteroides ovatus
tacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtgg

SEQ ID NO: 3 - DNA, 150 bp, Unknown
  Bacteria of family Ruminococcaceae
tacgtagggagcgagcgttgtccggaattactgggtgtaaagggagcgtaggcggggtctcaagtcgaatgttaaatctaccggctcaactggtagctgcgttcgaaactggggctcttgagtgaagtagaggcaggcggaattcctagt

SEQ ID NO: 4 - DNA, 150 bp, Unknown
  Bacteria of family Ruminococcaceae
tacgtagggagcgagcgttgtccggaattactgggtgtaaagggagcgtaggcgggaaagcaagttggaagtgaaatgcatgggcttaacccatgagctgctttcaaaactgtttttcttgagtgaagtagaggcaggcggaattcctag

SEQ ID NO: 5 - DNA, 150 bp, Unknown
  Bacteria of genus Holdemania
tacgtaggtggcgagcgttatccggaattattgggcgtaaagggtgcgcaggcggtttgttaagtttaaggtgaaagcgtggggcttaaccccatatagccttagaaactgacagactagagtacaggagagggcaatggaattccatgt

SEQ ID NO: 6 - DNA, 150 bp, Unknown
  Bacteria of family Lachnospiraceae
tacgtagggggcaagcgttatccggatttactgggtgtaaagggagcgtagacggcatggcaagccagatgtgaaaacccagggctcaaccttgggattgcatttggaactgtcaggctggagtgcaggagaggtaagcggaattcctag

SEQ ID NO: 7 - DNA, 150 bp, Unknown
  Bacteria of genus Parabacteroides
tacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtgg

SEQ ID NO: 8 - DNA, 150 bp, Unknown
  Bacteria of family Lachnospiraceae
tacgtagggggcaagcgttatccggatttactgggtgtaaagggagcgtagacggcatgacaagccagatgtgaaaacccagggctcaaccctgggactgcatttggaactgccaggctggagtgcaggagaggtaagcggaattcctag

SEQ ID NO: 9 - DNA, 150 bp, Unknown
  Bacteria of family Lachnospiraceae
tacgtagggggcaagcgttatccggatttactgggtgtaaagggagcgtagacggcacagcaagtctgaagtgaaatccccgggctcaacccgggaactgctttggaaactgttgggctggagtgctggagaggcaagcggaattcctag

SEQ ID NO: 10 - DNA, 150 bp, Unknown
  Bacteria of genus Bacteroides
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtgg

SEQ ID NO: 11 - DNA, 150 bp, Unknown
  Bacteria of family Rikenellaceae
tacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatgg

SEQ ID NO: 12 - DNA, 150 bp, Unknown
  Bacteria of family Ruminococcaceae
tacgtaggtggcaagcgttgtccggatttactgggtgtaaagggcgtgtaggcgggtagacaagtcagatgtgaaataccggggctcaactccggggctgcatttgaaactgtatatcttgagtgtcggagaggaaagcggaattcctag

SEQ ID NO: 13 - DNA, 150 bp, Unknown
  Bacteria of genus Paraprevotella
tacggaagatgcgagcgttatccggatttattgggtttaaagggagcgtaggcgggcttttaagtcagcggtcaaatgtcacggctcaaccgtggccagccgttgaaactgcaagccttgagtctgcacagggcacatggaattcgtggt

SEQ ID NO: 14 - DNA, 150 bp, Unknown
  Bacteria of genus Coprococcus
tacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctag

SEQ ID NO: 15 - DNA, 150 bp, Unknown
  Bacteria of genus Paraprevotella
tacggaagatgcgagcgttatccggatttattgggtttaaagggagcgtaggcgggcttttaagtcagcggtcaaatgtcgtggctcaaccatgtcaagccgttgaaactgtaagccttgagtctgcacagggcacatggaattcgtggt

SEQ ID NO: 16 - DNA, 150 bp, Unknown
  Bacteria of family Rikenellaceae
tacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatgg

SEQ ID NO: 17 - DNA, 120 bp, Unknown
  Bacteria of genus Bacteroides
atccggatttattgggtttaaagggagcgtaggtggacagttaagtcagttgtgaaagtttgcggctcaaccgtaaaattgcagttgatactggctgtcttgagtacagtagaggtgggc

SEQ ID NO: 18 - DNA, 120 bp, Eisenbergiella tayi
atccggatttattgggtttaaagggtgcgtaggtggtgatttaagtcagcggtgaaagtttgtggctcaaccataaaattgccgttgaaactgggttacttgagtgtgtttgaggtaggc

SEQ ID NO: 19 - DNA, 120 bp, Eisenbergiella tayi
atccggatttactgggtgtaaagggagcgtagacggcatggcaagccagatgtgaaaacccagggctcaaccttgggattgcatttggaactgccaggctggagtgcaggagaggtaagc

SEQ ID NO: 20 - DNA, 120 bp, Parabacteroides merdae
atccggatttactgggtgtaaagggagcgtagacggcatggcaagccagatgtgaaaacccagggctcaaccttgggattgcatttggaactgtcaggctggagtgcaggagaggtaagc